SEQUENCE LISTING (32 sequences)

SEQ ID NO: 1 - protein, 7 aa, Artificial Sequence
  Synthetic Construct
SNSAAWN

SEQ ID NO: 2 - protein, 18 aa, Artificial Sequence
  Synthetic Construct
KTYYRFKWYSDYAVSVKG

SEQ ID NO: 3 - protein, 14 aa, Artificial Sequence
  Synthetic Construct
ESTTYDLLAGPFDY

SEQ ID NO: 4 - protein, 17 aa, Artificial Sequence
  Synthetic Construct
KSSQTVLYSSNNKKYLA

SEQ ID NO: 5 - protein, 7 aa, Artificial Sequence
  Synthetic Construct
WASTRES

SEQ ID NO: 6 - protein, 9 aa, Artificial Sequence
  Synthetic Construct
QQYYSTPFT

SEQ ID NO: 7 - protein, 23 aa, Artificial Sequence
  Synthetic Construct
DIVMTQSPDSLAVSLGERATINC

SEQ ID NO: 8 - protein, 15 aa, Artificial Sequence
  Synthetic Construct
WYQQKPGQPPNLLIY

SEQ ID NO: 9 - protein, 32 aa, Artificial Sequence
  Synthetic Construct
GVPDRFSGSGSGTDFTLTISSLQAEDVAVYYC

SEQ ID NO: 10 - protein, 10 aa, Artificial Sequence
  Synthetic Construct
FGPGTKVEIK

SEQ ID NO: 11 - protein, 30 aa, Artificial Sequence
  Synthetic Construct
  MISC_FEATURE (1)..(1): Xaa is Gln or Glu
XVQLQQSGPGLVKPSQTLSLTCAISGDSVS

SEQ ID NO: 12 - protein, 14 aa, Artificial Sequence
  Synthetic Construct
WIRQSPSRGLEWLG

SEQ ID NO: 13 - protein, 32 aa, Artificial Sequence
  Synthetic Construct
RITINPDTSKNQFSLQLNSVTPEDTAVFYCTR

SEQ ID NO: 14 - protein, 11 aa, Artificial Sequence
  Synthetic Construct
WGQGTLVTVSS

SEQ ID NO: 15 - protein, 30 aa, Artificial Sequence
  Synthetic Construct
EVQLQQSGPGLVKPSQTLSLTCAISGDSVS

SEQ ID NO: 16 - protein, 30 aa, Artificial Sequence
  Synthetic Construct
QVQLQQSGPGLVKPSQTLSLTCAISGDSVS

SEQ ID NO: 17 - protein, 126 aa, Artificial Sequence
  Synthetic Construct
EVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 18 - protein, 126 aa, Artificial Sequence
  Synthetic Construct
QVQLQQSGPGLVKPSQTLSLTCAISGDSVSSNSAAWNWIRQSPSRGLEWLGKTYYRFKWYSDYAVSVKGRITINPDTSKNQFSLQLNSVTPEDTAVFYCTRESTTYDLLAGPFDYWGQGTLVTVSS

SEQ ID NO: 19 - protein, 113 aa, Artificial Sequence
  Synthetic Construct
DIVMTQSPDSLAVSLGERATINCKSSQTVLYSSNNKKYLAWYQQKPGQPPNLLIYWASTRESGVPDRFSGSGSGTDFTLTISSLQAEDVAVYYCQQYYSTPFTFGPGTKVEIK

SEQ ID NO: 20 - protein, 10 aa, Artificial Sequence
  Synthetic Construct
GFTFSDSWIH

SEQ ID NO: 21 - protein, 18 aa, Artificial Sequence
  Synthetic Construct
AWISPYGGSTYYADSVKG

SEQ ID NO: 22 - protein, 9 aa, Artificial Sequence
  Synthetic Construct
RHWPGGFDY

SEQ ID NO: 23 - protein, 11 aa, Artificial Sequence
  Synthetic Construct
RASQDVSTAVA

SEQ ID NO: 24 - protein, 7 aa, Artificial Sequence
  Synthetic Construct
SASFLYS

SEQ ID NO: 25 - protein, 9 aa, Artificial Sequence
  Synthetic Construct
QQYLYHPAT

SEQ ID NO: 26 - protein, 118 aa, Artificial Sequence [sequence omitted]

SEQ ID NO: 27 - protein, 108 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 28 - protein, 447 aa, Artificial Sequence
  Synthetic Construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 214 aa, Artificial Sequence
  Synthetic Construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 31 - protein, 223 aa, Homo sapiens [sequence omitted]

SEQ ID NO: 32 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

(19) United States
(12) Patent Application Publication
(10) Pub. No.: US 2020/0399376 A1
(43) Pub. Date: Dec. 24, 2020

(54) DOSING FOR TREATMENT WITH ANTI-TIGIT AND ANTI-PD-L1 ANTAGONIST ANTIBODIES

(71) Applicant: Genentech, Inc., South San Francisco, CA (US)

(72) Inventors: Raymond D. MENG, Mountain View, CA (US); Sean Keith KELLEY, Burlingame, CA (US); Namrata Srivastava PATIL, Sunnyvale, CA (US)

(21) Appl. No.: 16/975,549

(22) PCT Filed: Feb. 26, 2019
(86) PCT No.: PCT/US2019/019603

§ 371 Date: Aug. 25, 2020

Related U.S. Application Data
(60) Provisional application No. 62/807,469, filed on Feb. 19, 2019.
(60) Provisional application No. 62/641,919, filed on Mar. 12, 2018.
(60) Provisional application No. 62/635,484, filed on Feb. 26, 2018.

(51) Int. Cl. C07K 16/28; G01N 33/50; C12N 15/10; C12Q 1/6825

(52) U.S. Cl. CPC C07K 16/2827; G01N 33/5005; C12N 15/1096; C12Q 1/6825; A61K 2039/545; C07K 2317/54; C07K 2317/55; C07K 2317/622; C07K 2317/76; C07K 2317/567

(57) ABSTRACT

The invention provides methods of dosing for the treatment of cancers. In particular, provided are methods for treating human patients having lung cancer, such as non-small cell lung cancer (NSCLC), by administering a combination of an anti-TIGIT antagonist antibody and an anti-PD-L1 antagonist antibody.

### SEQUENCE LISTING

The instant application contains a Sequence Listing which has been submitted electronically in ASCII format and is hereby incorporated by reference in its entirety. Said ASCII copy, created on Aug. 24, 2020, is named 50474-183004_Sequence_Listing_8_24_20_ST25 and is 24,253 bytes in size.

### FIELD OF THE INVENTION

The present invention relates to the treatment of cancer (e.g., lung cancer). More specifically, the invention concerns the treatment of patients having cancer (e.g., lung cancer) by administering a combination of an anti-T-cell immunoreceptor with Ig and ITIM domains (TIGIT) antagonist antibody and an anti-programmed death ligand-1 (PD-L1) antagonist antibody.

### BACKGROUND OF THE INVENTION

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. Cancer care thus represents a significant and ever-increasing societal burden.

Lung cancer, in particular, remains the leading cause of cancer deaths worldwide, accounting for approximately 13% of all new cancers in 2012. In 2017 in the United States, it was estimated that there were 222,500 new cases of lung cancer and 155,870 lung cancer deaths. Non-small cell lung cancer (NSCLC) is the predominant subtype, accounting for approximately 85% of all cases. The overall five-year survival rate for advanced disease is 2%-4%. Poor prognostic factors for survival in patients with NSCLC include advanced stage of disease at the time of initial diagnosis, poor performance status, and a history of unintentional weight loss. More than half of the patients with NSCLC are diagnosed with distant disease, which directly contributes to poor survival prospects.

Despite improvements in the first-line treatment of patients with advanced NSCLC that have resulted in longer survival times and reduced disease-related symptoms, nearly all patients experience disease progression. Cancer immunotherapies in particular offer the possibility of long-term disease control. In the second-line metastatic NSCLC setting, PD-L1/PD-1 blocking antibodies (e.g., atezolizumab, nivolumab, and pembrolizumab) provided clinically meaningful benefit in either unselected or PD-L1-selected advanced NSCLC patients; however, a substantial proportion of patients still remained unresponsive or progressed on anti-PD-L1/PD-1 treatment, and the escape mechanisms to such treatment are poorly understood.

Thus, there is an unmet need in the field for the development of efficacious immunotherapies and methods of dosing the same for the treatment of cancers (e.g., lung cancer, e.g., NSCLC) that achieve a more favorable benefit-risk profile.

### SUMMARY OF THE INVENTION

The present invention relates to methods of treating a subject having cancer (e.g., lung cancer, e.g., NSCLC, e.g., squamous or non-squamous NSCLC, e.g., locally advanced unresectable NSCLC (e.g., Stage IIIB NSCLC), or recurrent or metastatic NSCLC (e.g., Stage IV NSCLC)) by administering a combination of an anti-TIGIT antagonist antibody (e.g., an anti-TIGIT antagonist antibody as disclosed herein, e.g., tiragolumab) and an anti-PD-L1 antagonist antibody (e.g., atezolizumab).

In a first aspect, the invention features a method for treating a subject having a lung cancer comprising administering to the subject one or more dosing cycles of an anti-TIGIT antagonist antibody at a fixed dose of between about 30 mg to about 1200 mg every three weeks and an anti-PD-L1 antagonist antibody at a fixed dose of between about 80 mg to about 1600 mg every three weeks.

In some embodiments of the first aspect, the method comprises administering to the subject an anti-TIGIT antagonist antibody at a fixed dose of between about 30 mg to about 600 mg every three weeks. In some embodiments, the method comprises administering to the subject an anti-TIGIT antagonist antibody at a fixed dose of about 600 mg every three weeks.

In some embodiments of the first aspect, the anti-TIGIT antagonist antibody comprises the following hypervariable regions (HVRs): an HVR-H1 sequence comprising the amino acid sequence of SNSAAWN (SEQ ID NO: 1); an HVR-H2 sequence comprising the amino acid sequence of KTYYRFKWYSDYAVSVKG (SEQ ID NO: 2); an HVR-H3 sequence comprising the amino acid sequence of ESTTYDLLAGPFDY (SEQ ID NO: 3); an HVR-L1 sequence comprising the amino acid sequence of KSSQTVLYSSNNKKYLA (SEQ ID NO: 4); an HVR-L2 sequence comprising the amino acid sequence of WASTRES (SEQ ID NO: 5); and an HVR-L3 sequence comprising the amino acid sequence of QQYYSTPFT (SEQ ID NO: 6). In some embodiments, the anti-TIGIT antagonist antibody further comprises the following light chain variable region framework regions (FRs): an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and an FR-L4 comprising the amino acid sequence of FGPGTKVEIK (SEQ ID NO: 10). In some embodiments, the anti-TIGIT antagonist antibody further comprises the following heavy chain variable region FRs: an FR-H1 comprising the amino acid sequence of X₁VQLQQSGPGLVKPSQTLSLTCAISGDSVS (SEQ ID NO: 11), wherein X₁ is Q or E; an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14). In some embodiments, X₁ is Q. In some embodiments, X₁ is E.

In some embodiments of the first aspect, the anti-TIGIT antagonist antibody comprises: (a) a heavy chain variable (VH) domain comprising an amino acid sequence having at least 95% sequence identity to the amino acid sequence of SEQ ID NO: 17 or 18; (b) a light chain variable (VL) domain comprising an amino acid sequence having at least 95% sequence identity to the amino acid sequence of SEQ ID NO: 19; or (c) a VH domain as in (a) and a VL domain as in (b).

In some embodiments of the first aspect, the anti-TIGIT antagonist antibody comprises: a VH domain comprising the amino acid sequence of SEQ ID NO: 17 or 18 and a VL domain comprising the amino acid sequence of SEQ ID NO: 19.

In some embodiments of the first aspect, the anti-TIGIT antagonist antibody is a monoclonal antibody. In some embodiments, the anti-TIGIT antagonist antibody is a human antibody (e.g., a monoclonal human antibody).

In some embodiments of the first aspect, the anti-TIGIT antagonist antibody is a full-length antibody. In some embodiments of the first aspect, the anti-TIGIT antagonist antibody is tiragolumab.

In some embodiments of the first aspect, the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

In some embodiments of the first aspect, the anti-TIGIT antagonist antibody is an IgG class antibody. In some embodiments, the IgG class antibody is an IgG1 subclass antibody.

In some embodiments of the first aspect, the method comprises administering to the subject an anti-PD-L1 antibody at a fixed dose of about 1200 mg every three weeks.

In some embodiments of the first aspect, the anti-PD-L1 antagonist antibody is atezolizumab (MPDL3280A), YW243.55.S70, MSB0010718C, MDX-1105, or MEDI4736. In some embodiments, the anti-PD-L1 antagonist antibody is atezolizumab.

In some embodiments of the first aspect, the anti-PD-L1 antagonist antibody comprises the following HVRs: an HVR-H1 sequence comprising the amino acid sequence of GFTFSDSWIH (SEQ ID NO: 20); an HVR-H2 sequence comprising the amino acid sequence of AWISPYGGSTYYADSVKG (SEQ ID NO: 21); an HVR-H3 sequence comprising the amino acid sequence of RHWPGGFDY (SEQ ID NO: 22); an HVR-L1 sequence comprising the amino acid sequence of RASQDVSTAVA (SEQ ID NO: 23); an HVR-L2 sequence comprising the amino acid sequence of SASFLYS (SEQ ID NO: 24); and an HVR-L3 sequence comprising the amino acid sequence of QQYLYHPAT (SEQ ID NO: 25). In some embodiments, the anti-PD-L1 antagonist antibody comprises: (a) a heavy chain variable (VH) domain comprising an amino acid sequence having at least 95% sequence identity to the amino acid sequence of SEQ ID NO: 26; (b) a light chain variable (VL) domain comprising an amino acid sequence having at least 95% sequence identity to the amino acid sequence of SEQ ID NO: 27; or (c) a VH domain as in (a) and a VL domain as in (b).

In some embodiments of the first aspect, the anti-PD-L1 antagonist antibody comprises: a VH domain comprising the amino acid sequence of SEQ ID NO: 26 and a VL domain comprising the amino acid sequence of SEQ ID NO: 27.

In some embodiments of the first aspect, the anti-PD-L1 antagonist antibody is a monoclonal antibody. In some embodiments, the anti-PD-L1 antagonist antibody is a humanized antibody (e.g., a monoclonal humanized antibody).

In some embodiments of the first aspect, the anti-PD-L1 antagonist antibody is a full-length antibody.

In some embodiments of the first aspect, the anti-PD-L1 antagonist antibody is an antibody fragment that binds PD-L1 selected from the group consisting of Fab, Fab′, Fab′-SH, Fv, single chain variable fragment (scFv), and (Fab′)2 fragments.

In some embodiments of the first aspect, the anti-PD-L1 antagonist antibody is an IgG class antibody. In some embodiments, the IgG class antibody is an IgG1 subclass antibody.

In some embodiments of the first aspect, the method comprises administering to the subject the anti-TIGIT antagonist antibody at a fixed dose of about 600 mg every three weeks and the anti-PD-L1 antagonist antibody at a fixed dose of about 1200 mg every three weeks.

In some embodiments of the first aspect, the length of each of the one or more dosing cycles is 21 days.

In some embodiments of the first aspect, the method comprises administering to the subject the anti-TIGIT antagonist antibody and the anti-PD-L1 antagonist antibody on about Day 1 of each of the one or more dosing cycles.

In some embodiments of the first aspect, the method comprises administering to the subject the anti-TIGIT antagonist antibody before the anti-PD-L1 antagonist antibody. In some embodiments, the method comprises a first observation period following administration of the anti-TIGIT antagonist antibody and second observation period following administration of the anti-PD-L1 antagonist antibody. In some embodiments, the first observation period and the second observation period are each between about 30 minutes to about 60 minutes in length.

In some embodiments of the first aspect, the method comprises administering to the subject the anti-PD-L1 antagonist antibody before the anti-TIGIT antagonist antibody. In some embodiments, the method comprises a first observation period following administration of the anti-PD-L1 antagonist antibody and second observation period following administration of the anti-TIGIT antagonist antibody. In some embodiments, the first observation period and the second observation period are each between about 30 minutes to about 60 minutes in length.

In some embodiments of the first aspect, the method comprises administering to the subject the anti-TIGIT antagonist antibody and the anti-PD-L1 antagonist antibody simultaneously.

In some embodiments of the first aspect, the method comprises administering to the subject the anti-TIGIT antagonist antibody and anti-PD-L1 antagonist antibody intravenously. In some embodiments, the method comprises administering to the subject the anti-TIGIT antagonist antibody by intravenous infusion over 60±10 minutes. In some embodiments, the method comprises administering to the subject the anti-PD-L1 antagonist antibody by intravenous infusion over 60±15 minutes.

In some embodiments of the first aspect, a tumor sample obtained from the subject has been determined to have a detectable expression level of PD-L1.

In some embodiments of the first aspect, the detectable expression level of PD-L1 is a detectable protein expression level of PD-L1. In some embodiments, the detectable protein expression level of PD-L1 has been determined by an immunohistochemical (IHC) assay. In some embodiments, the IHC assay uses anti-PD-L1 antibody 22C3, SP142, SP263, or 28-8.

In some embodiments of the first aspect, the IHC assay uses anti-PD-L1 antibody 22C3. In some embodiments, the tumor sample has been determined to have a tumor proportion score (TPS) of greater than, or equal to, 1%. In some embodiments, the TPS is greater than, or equal to, 1% and less than 50%. In some embodiments, the TPS is greater than, or equal to, 50%.

In some embodiments of the first aspect, the IHC assay uses anti-PD-L1 antibody SP142. In some embodiments, the tumor sample has been determined to have a detectable expression level of PD-L1 in greater than, or equal to, 1% of the tumor cells in the tumor sample. In some embodiments, the tumor sample has been determined to have a detectable expression level of PD-L1 in greater than, or equal to, 1% and less than 5% of the tumor cells in the tumor sample. In some embodiments, the tumor sample has been determined to have a detectable expression level of PD-L1 in greater than, or equal to, 5% and less than 50% of the tumor cells in the tumor sample. In some embodiments, the tumor sample has been determined to have a detectable expression level of PD-L1 in greater than, or equal to, 50% of the tumor cells in the tumor sample. In some embodiments, the tumor sample has been determined to have a detectable expression level of PD-L1 in tumor-infiltrating immune cells that comprise greater than, or equal to, 1% of the tumor sample. In some embodiments, the tumor sample has been determined to have a detectable expression level of PD-L1 in tumor-infiltrating immune cells that comprise greater than, or equal to, 1% and less than 5% of the tumor sample. In some embodiments, the tumor sample has been determined to have a detectable expression level of PD-L1 in tumor-infiltrating immune cells that comprise greater than, or equal to, 5% and less than 10% of the tumor sample. In some embodiments, the tumor sample has been determined to have a detectable expression level of PD-L1 in tumor-infiltrating immune cells that comprise greater than, or equal to, 10% of the tumor sample.

In some embodiments of the first aspect, the detectable expression level of PD-L1 is a detectable nucleic acid expression level of PD-L1. In some embodiments, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

In some embodiments of the first aspect, the lung cancer is a non-small cell lung cancer (NSCLC). In some embodiments, the NSCLC is a squamous NSCLC. In some embodiments, the NSCLC is a non-squamous NSCLC. In some embodiments, the NSCLC is a locally advanced unresectable NSCLC. In some embodiments, the NSCLC is a Stage IIIB NSCLC. In some embodiments, the NSCLC is a recurrent or metastatic NSCLC. In some embodiments, the NSCLC is a Stage IV NSCLC. In some embodiments, the subject has not been previously treated for Stage IV NSCLC.

In some embodiments of the first aspect, the subject does not have a sensitizing epidermal growth factor receptor (EGFR) gene mutation or anaplastic lymphoma kinase (ALK) gene rearrangement.

In some embodiments of the first aspect, the subject does not have a pulmonary lymphoepithelioma-like carcinoma subtype of NSCLC.

In some embodiments of the first aspect, the subject does not have an active Epstein-Barr virus (EBV) infection or a known or suspected chronic active EBV infection. In some embodiments, the subject is negative for EBV IgM or negative by EBV PCR. In some embodiments, the subject is negative for EBV IgM and negative by EBV PCR. In some embodiments, the subject is positive for EBV IgG or positive for Epstein-Barr nuclear antigen (EBNA). In some embodiments, the subject is positive for EBV IgG and positive for EBNA.

In some embodiments of the first aspect, the subject is negative for EBV IgG or negative for EBNA. In some embodiments, the subject is negative for EBV IgG and negative for EBNA.

In some embodiments of the first aspect, the treating results in a clinical response. In some embodiments, the clinical response is an increase in the objective response rate (ORR) of the subject as compared to a reference ORR. In some embodiments, the reference ORR is the median ORR of a population of subjects who have received a treatment comprising an anti-PD-L1 antagonist antibody without an anti-TIGIT antagonist antibody. In some embodiments, the clinical response is an increase in the progression-free survival (PFS) of the subject as compared to a reference PFS time. In some embodiments, the reference PFS time is the median PFS time of a population of subjects who have received a treatment comprising an anti-PD-L1 antagonist antibody without an anti-TIGIT antagonist antibody.

In a second aspect, the invention features a method for treating a subject having a NSCLC comprising administering to the subject one or more dosing cycles of an anti-TIGIT antagonist antibody at a fixed dose of 600 mg every three weeks and atezolizumab at a fixed dose of 1200 mg every three weeks, wherein the anti-TIGIT antagonist antibody comprises: a VH domain comprising the amino acid sequence of SEQ ID NO: 17 or 18 and a VL domain comprising the amino acid sequence of SEQ ID NO: 19.

In a third aspect, the invention features a method for treating a subject having a NSCLC comprising (a) obtaining a tumor sample from the subject; (b) detecting the protein expression level of PD-L1 in the tumor sample by an IHC assay using anti-PD-L1 antibody 22C3 and determining a TPS therefrom; (c) identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1% and less than 50%, wherein the anti-TIGIT antagonist antibody comprises: a VH domain comprising the amino acid sequence of SEQ ID NO: 17 or 18 and a VL domain comprising the amino acid sequence of SEQ ID NO: 19; and (d) administering to the identified subject the therapy.

In a fourth aspect, the invention features a method for treating a subject having a NSCLC comprising (a) obtaining a tumor sample from the subject; (b) detecting the protein expression level of PD-L1 in the tumor sample by an IHC assay using anti-PD-L1 antibody 22C3 and determining a TPS therefrom; (c) identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 50%, wherein the anti-TIGIT antagonist antibody comprises: a VH domain comprising the amino acid sequence of SEQ ID NO: 17 or 18 and a VL domain comprising the amino acid sequence of SEQ ID NO: 19; and (d) administering to the identified subject the therapy.

In a fifth aspect, the invention features a method of selecting a therapy for a subject having a NSCLC comprising (a) determining a TPS from a tumor sample from the subject by an IHC assay using anti-PD-L1 antibody 22C3; and (b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1% and less than 50%, wherein the anti-TIGIT antagonist antibody comprises: a VH domain comprising the amino acid sequence of SEQ ID NO: 17 or 18 and a VL domain comprising the amino acid sequence of SEQ ID NO: 19.

In a sixth aspect, the invention features a method of selecting a therapy for a subject having a NSCLC comprising (a) determining a TPS from a tumor sample from the subject by an IHC assay using anti-PD-L1 antibody 22C3; and (b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 50%, wherein the anti-TIGIT antagonist antibody comprises: a VH domain comprising the amino acid sequence of SEQ ID NO: 17 or 18 and a VL domain comprising the amino acid sequence of SEQ ID NO: 19.

In a seventh aspect, the invention features a method for treating a subject having a NSCLC comprising administering to the subject one or more dosing cycles of tiragolumab at a fixed dose of 600 mg every three weeks and atezolizumab at a fixed dose of 1200 mg every three weeks.

In an eighth aspect, the invention features a method for treating a subject having a NSCLC comprising (a) obtaining a tumor sample from the subject; (b) detecting the protein expression level of PD-L1 in the tumor sample by an IHC assay using anti-PD-L1 antibody 22C3 and determining a TPS therefrom; (c) identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1% and less than 50%; and (d) administering to the identified subject the therapy.

In a ninth aspect, the invention features a method for treating a subject having a NSCLC comprising (a) obtaining a tumor sample from the subject; (b) detecting the protein expression level of PD-L1 in the tumor sample by an IHC assay using anti-PD-L1 antibody 22C3 and determining a TPS therefrom; (c) identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 50%; and (d) administering to the identified subject the therapy.

In a tenth aspect, the invention features a method of selecting a therapy for a subject having a NSCLC comprising (a) determining a TPS from a tumor sample from the subject by an IHC assay using anti-PD-L1 antibody 22C3; and (b) selecting for the subject a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1% and less than 50%.

In an eleventh aspect, the invention features a method of selecting a therapy for a subject having a NSCLC comprising (a) determining a TPS from a tumor sample from the subject by an IHC assay using anti-PD-L1 antibody 22C3; and (b) selecting for the subject a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 50%.

In a twelfth aspect, the invention features a method of selecting a therapy for a subject having a NSCLC, the method comprising (a) detecting the mutational status of the epidermal growth factor receptor (EGFR) gene and anaplastic lymphoma kinase (ALK) gene from a sample from the subject and detecting the absence of a sensitizing EGFR gene mutation or ALK gene rearrangement; and (b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks, based on the subject not having a sensitizing EGFR gene mutation or ALK gene rearrangement, wherein the anti-TIGIT antagonist antibody comprises a VH domain comprising the amino acid sequence of SEQ ID NO: 17 or 18; and a VL domain comprising the amino acid sequence of SEQ ID NO: 19.

In a thirteenth aspect, the invention features a method of selecting a therapy for a subject having a NSCLC, the method comprising (a) biopsying a tumor sample from the subject and detecting a subtype of the NSCLC other than a pulmonary lymphoepithelioma-like carcinoma; and (b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks, based on the subject not having a pulmonary lymphoepithelioma-like carcinoma subtype of NSCLC, wherein the anti-TIGIT antagonist antibody comprises a VH domain comprising the amino acid sequence of SEQ ID NO: 17 or 18; and a VL domain comprising the amino acid sequence of SEQ ID NO: 19.

In a fourteenth aspect, the invention features a method of selecting a therapy for a subject having a NSCLC, the method comprising (a) detecting the presence of one or more of Epstein-Barr virus (EBV) IgM, EBV IgG, Epstein-Barr nuclear antigen (EBNA), and Epstein-Barr viral particles in a sample from the subject, and (b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks, on based on the subject being (i) negative for EBV IgG and/or EBNA; or (ii) positive for EBV IgG and/or EBNA, and negative for both EBV IgM and Epstein-Barr viral particles, wherein the anti-TIGIT antagonist antibody comprises a VH domain comprising the amino acid sequence of SEQ ID NO: 17 or 18; and a VL domain comprising the amino acid sequence of SEQ ID NO: 19.

In a fifteenth aspect, the invention features a method of selecting a therapy for a subject having a NSCLC, the method comprising (a) detecting the mutational status of the epidermal growth factor receptor (EGFR) gene and anaplastic lymphoma kinase (ALK) gene from a sample from the subject and detecting the absence of a sensitizing EGFR gene mutation or ALK gene rearrangement; and (b) selecting for the subject a therapy comprising one or more dosing cycles of tiragolumab administered at a fixed dose of 600 mg every three weeks and atezolizumab administered at a fixed dose of 1200 mg every three weeks, based on the subject not having a sensitizing EGFR gene mutation or ALK gene rearrangement.

In a sixteenth aspect, the invention features a method of selecting a therapy for a subject having a NSCLC, the method comprising (a) biopsying a tumor sample from the subject and detecting a subtype of the NSCLC other than a pulmonary lymphoepithelioma-like carcinoma; and (b) selecting for the subject a therapy comprising one or more dosing cycles of tiragolumab administered at a fixed dose of 600 mg every three weeks and atezolizumab administered at a fixed dose of 1200 mg every three weeks, based on the subject not having a pulmonary lymphoepithelioma-like carcinoma subtype of NSCLC.

In a seventeenth aspect, the invention features a method of selecting a therapy for a subject having a NSCLC, the method comprising (a) detecting the presence of one or more of Epstein-Barr virus (EBV) IgM, EBV IgG, Epstein-Barr nuclear antigen (EBNA), and Epstein-Barr viral particles in a sample from the subject, and (b) selecting for the subject a therapy comprising one or more dosing cycles of tiragolumab administered at a fixed dose of 600 mg every three weeks and atezolizumab administered at a fixed dose of 1200 mg every three weeks, on based on the subject being (i) negative for EBV IgG and/or EBNA; or (ii) positive for EBV IgG and/or EBNA, and negative for both EBV IgM and Epstein-Barr viral particles.

In some embodiments of any of the second, third, fourth, fifth, sixth, seventh, eighth, ninth, tenth, eleventh, thirteenth, fourteenth, sixteenth, and seventeenth aspects, the subject does not have a sensitizing epidermal growth factor receptor (EGFR) gene mutation or anaplastic lymphoma kinase (ALK) gene rearrangement.

In some embodiments of any of the second, third, fourth, fifth, sixth, seventh, eighth, ninth, tenth, eleventh, twelfth, fourteenth, fifteenth, and seventeenth aspects, the subject does not have a pulmonary lymphoepithelioma-like carcinoma subtype of NSCLC.

In some embodiments of any of the second, third, fourth, fifth, sixth, seventh, eighth, ninth, tenth, eleventh, twelfth, thirteenth, fifteenth, and sixteenth aspects, the subject does not have an active EBV infection or a known or suspected chronic active EBV infection. In some embodiments, the subject is negative for EBV IgM or negative by EBV PCR. In some embodiments, the subject is negative for EBV IgM and negative by EBV PCR. In some embodiments, the subject is positive for EBV IgG or positive for EBNA. In some embodiments, the subject is positive for EBV IgG and positive for EBNA.

In some embodiments of any of the second, third, fourth, fifth, sixth, seventh, eighth, ninth, tenth, eleventh, twelfth, thirteenth, fifteenth, and sixteenth aspects, the subject is negative for EBV IgG or negative for EBNA. In some embodiments, the subject is negative for EBV IgG and negative for EBNA.

In an eighteenth aspect, the invention features an anti-TIGIT antagonist antibody and an anti-PD-L1 antagonist antibody for use in a method of treating a subject having a lung cancer, the method comprising administering to the subject one or more dosing cycles of the anti-TIGIT antagonist antibody at a fixed dose of between about 30 mg to about 1200 mg every three weeks and the anti-PD-L1 antagonist antibody at a fixed dose of between about 80 mg to about 1600 mg every three weeks.

In some embodiments of the eighteenth aspect, the anti-TIGIT antagonist antibody is to be administered to the subject at a fixed dose of between about 30 mg to about 600 mg every three weeks. In some embodiments, the anti-TIGIT antagonist antibody is to be administered to the subject at a fixed dose of about 600 mg every three weeks.

In some embodiments of the eighteenth aspect, the anti-TIGIT antagonist antibody comprises the following HVRs: an HVR-H1 sequence comprising the amino acid sequence of SNSAAWN (SEQ ID NO: 1); an HVR-H2 sequence comprising the amino acid sequence of KTYYRFKWYSDYAVSVKG (SEQ ID NO: 2); an HVR-H3 sequence comprising the amino acid sequence of ESTTYDLLAGPFDY (SEQ ID NO: 3); an HVR-L1 sequence comprising the amino acid sequence of KSSQTVLYSSNNKKYLA (SEQ ID NO: 4); an HVR-L2 sequence comprising the amino acid sequence of WASTRES (SEQ ID NO: 5); and an HVR-L3 sequence comprising the amino acid sequence of QQYYSTPFT (SEQ ID NO: 6). In some embodiments, the anti-TIGIT antagonist antibody further comprises the following light chain variable region FRs: an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and an FR-L4 comprising the amino acid sequence of FGPGTKVEIK (SEQ ID NO: 10). In some embodiments, the anti-TIGIT antagonist antibody further comprises the following heavy chain variable region FRs: an FR-H1 comprising the amino acid sequence of X₁VQLQQSGPGLVKPSQTLSLTCAISGDSVS (SEQ ID NO: 11), wherein X₁ is Q or E; an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14). In some embodiments, X₁ is Q. In some embodiments, X₁ is E.

In some embodiments of the eighteenth aspect, the anti-TIGIT antagonist antibody comprises: (a) a heavy chain variable (VH) domain comprising an amino acid sequence having at least 95% sequence identity to the amino acid sequence of SEQ ID NO: 17 or 18; (b) a light chain variable (VL) domain comprising an amino acid sequence having at least 95% sequence identity to the amino acid sequence of SEQ ID NO: 19; or (c) a VH domain as in (a) and a VL domain as in (b).

In some embodiments of the eighteenth aspect, the anti-TIGIT antagonist antibody comprises: a VH domain comprising the amino acid sequence of SEQ ID NO: 17 or 18 and a VL domain comprising the amino acid sequence of SEQ ID NO: 19.

In some embodiments of the eighteenth aspect, the anti-TIGIT antagonist antibody is a monoclonal antibody. In some embodiments, the anti-TIGIT antagonist antibody is a human antibody (e.g., a monoclonal human antibody).

In some embodiments of the eighteenth aspect, the anti-TIGIT antagonist antibody is a full-length antibody. In some embodiments of the eighteenth aspect, the anti-TIGIT antagonist antibody is tiragolumab.

In some embodiments of the eighteenth aspect, the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

In some embodiments of the eighteenth aspect, the anti-TIGIT antagonist antibody is an IgG class antibody. In some embodiments, the IgG class antibody is an IgG1 subclass antibody.

In some embodiments of the eighteenth aspect, anti-PD-L1 antagonist antibody is to be administered to the subject at a fixed dose of about 1200 mg every three weeks.

In some embodiments of the eighteenth aspect, the anti-PD-L1 antagonist antibody is atezolizumab (MPDL3280A), YW243.55.S70, MSB0010718C, MDX-1105, or MEDI4736. In some embodiments, the anti-PD-L1 antagonist antibody is atezolizumab.

In some embodiments of the eighteenth aspect, the anti-PD-L1 antagonist antibody comprises the following HVRs: an HVR-H1 sequence comprising the amino acid sequence of GFTFSDSWIH (SEQ ID NO: 20); an HVR-H2 sequence comprising the amino acid sequence of AWISPYGGSTYYADSVKG (SEQ ID NO: 21); an HVR-H3 sequence comprising the amino acid sequence of RHWPGGFDY (SEQ ID NO: 22); an HVR-L1 sequence comprising the amino acid sequence of RASQDVSTAVA (SEQ ID NO: 23); an HVR-L2 sequence comprising the amino acid sequence of SASFLYS (SEQ ID NO: 24); and an HVR-L3 sequence comprising the amino acid sequence of QQYLYHPAT (SEQ ID NO: 25). In some embodiments, the anti-PD-L1 antagonist antibody comprises: (a) a heavy chain variable (VH) domain comprising an amino acid sequence having at least 95% sequence identity to the amino acid sequence of SEQ ID NO: 26; (b) a light chain variable (VL) domain comprising an amino acid sequence having at least 95% sequence identity to the amino acid sequence of SEQ ID NO: 27; or (c) a VH domain as in (a) and a VL domain as in (b).

In some embodiments of the eighteenth aspect, the anti-PD-L1 antagonist antibody comprises: a VH domain comprising the amino acid sequence of SEQ ID NO: 26 and a VL domain comprising the amino acid sequence of SEQ ID NO: 27.

In some embodiments of the eighteenth aspect, the anti-PD-L1 antagonist antibody is a monoclonal antibody. In some embodiments, the anti-PD-L1 antagonist antibody is a humanized antibody (e.g., a monoclonal humanized antibody).

In some embodiments of the eighteenth aspect, the anti-PD-L1 antagonist antibody is a full-length antibody.

In some embodiments of the eighteenth aspect, the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

In some embodiments of the eighteenth aspect, the anti-PD-L1 antagonist antibody is an IgG class antibody. In some embodiments, the IgG class antibody is an IgG1 subclass antibody.

In some embodiments of the eighteenth aspect, the anti-TIGIT antagonist antibody is to be administered to the subject at a fixed dose of about 600 mg every three weeks and the anti-PD-L1 antagonist antibody is to be administered to the subject at a fixed dose of about 1200 mg every three weeks.

In some embodiments of the eighteenth aspect, the length of each of the one or more dosing cycles is 21 days.

In some embodiments of the eighteenth aspect, the anti-TIGIT antagonist antibody and anti-PD-L1 antagonist antibody are to be administered to the subject on about Day 1 of each of the one or more dosing cycles.

In some embodiments of the eighteenth aspect, the anti-TIGIT antagonist antibody is to be administered to the subject before the anti-PD-L1 antagonist antibody. In some embodiments, a first observation period is to follow administration of the anti-TIGIT antagonist antibody and second observation period is to follow administration of the anti-PD-L1 antagonist antibody. In some embodiments, the first observation period and the second observation period are each between about 30 minutes to about 60 minutes in length.

In some embodiments of the eighteenth aspect, the anti-PD-L1 antagonist antibody is to be administered to the subject before the anti-TIGIT antagonist antibody. In some embodiments, a first observation period is to follow administration of the anti-PD-L1 antagonist antibody and second observation period is to follow administration of the anti-TIGIT antagonist antibody. In some embodiments, the first observation period and the second observation period are each between about 30 minutes to about 60 minutes in length.

In some embodiments of the eighteenth aspect, the anti-TIGIT antagonist antibody is to be administered to the subject simultaneously with the anti-PD-L1 antagonist antibody.

In some embodiments of the eighteenth aspect, the anti-TIGIT antagonist antibody and anti-PD-L1 antagonist antibody are to be administered to the subject intravenously. In some embodiments, the anti-TIGIT antagonist antibody is to be administered to the subject by intravenous infusion over 60±10 minutes.

In some embodiments, the anti-PD-L1 antagonist antibody is to be administered to the subject by intravenous infusion over 60±15 minutes.

In some embodiments of the eighteenth aspect, a tumor sample obtained from the subject has been determined to have a detectable expression level of PD-L1.

In some embodiments of the eighteenth aspect, the detectable expression level of PD-L1 is a detectable protein expression level of PD-L1. In some embodiments, the detectable protein expression level of PD-L1 has been determined by an immunohistochemical (IHC) assay. In some embodiments, the IHC assay uses anti-PD-L1 antibody 22C3, SP142, SP263, or 28-8.

In some embodiments of the eighteenth aspect, the IHC assay uses anti-PD-L1 antibody 22C3. In some embodiments, the tumor sample has been determined to have a tumor proportion score (TPS) of greater than, or equal to, 1%. In some embodiments, the TPS is greater than, or equal to, 1% and less than 50%. In some embodiments, the TPS is greater than, or equal to, 50%.

In some embodiments of the eighteenth aspect, the IHC assay uses anti-PD-L1 antibody SP142. In some embodiments, the tumor sample has been determined to have a detectable expression level of PD-L1 in greater than, or equal to, 1% of the tumor cells in the tumor sample. In some embodiments, the tumor sample has been determined to have a detectable expression level of PD-L1 in greater than, or equal to, 1% and less than 5% of the tumor cells in the tumor sample. In some embodiments, the tumor sample has been determined to have a detectable expression level of PD-L1 in greater than, or equal to, 5% and less than 50% of the tumor cells in the tumor sample. In some embodiments, the tumor sample has been determined to have a detectable expression level of PD-L1 in greater than, or equal to, 50% of the tumor cells in the tumor sample. In some embodiments, the tumor sample has been determined to have a detectable expression level of PD-L1 in tumor-infiltrating immune cells that comprise greater than, or equal to, 1% of the tumor sample. In some embodiments, the tumor sample has been determined to have a detectable expression level of PD-L1 in tumor-infiltrating immune cells that comprise greater than, or equal to, 1% and less than 5% of the tumor sample. In some embodiments, the tumor sample has been determined to have a detectable expression level of PD-L1 in tumor-infiltrating immune cells that comprise greater than, or equal to, 5% and less than 10% of the tumor sample. In some embodiments, the tumor sample has been determined to have a detectable expression level of PD-L1 in tumor-infiltrating immune cells that comprise greater than, or equal to, 10% of the tumor sample.

In some embodiments of the eighteenth aspect, the detectable expression level of PD-L1 is a detectable nucleic acid expression level of PD-L1. In some embodiments, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

In some embodiments of the eighteenth aspect, the lung cancer is a non-small cell lung cancer (NSCLC). In some embodiments, the NSCLC is a squamous NSCLC. In some embodiments, the NSCLC is a non-squamous NSCLC. In some embodiments, the NSCLC is a locally advanced unresectable NSCLC. In some embodiments, the NSCLC is a Stage IIIB NSCLC. In some embodiments, the NSCLC is a recurrent or metastatic NSCLC. In some embodiments, the NSCLC is a Stage IV NSCLC. In some embodiments, the subject has not been previously treated for Stage IV NSCLC.

In some embodiments of the eighteenth aspect, the subject does not have a sensitizing epidermal growth factor receptor (EGFR) gene mutation or anaplastic lymphoma kinase (ALK) gene rearrangement.

In some embodiments of the seventh aspect, the subject does not have a pulmonary lymphoepithelioma-like carcinoma subtype of NSCLC.

In some embodiments of the eighteenth aspect, the subject does not have an active EBV infection or a known or suspected chronic active EBV infection. In some embodiments, the subject is negative for EBV IgM or negative by EBV PCR. In some embodiments, the subject is negative for EBV IgM and negative by EBV PCR. In some embodiments, the subject is positive for EBV IgG or positive for EBNA. In some embodiments, the subject is positive for EBV IgG and positive for EBNA.

In some embodiments of the eighteenth aspect, the subject is negative for EBV IgG or negative for EBNA. In some embodiments, the subject is negative for EBV IgG and negative for EBNA.

In some embodiments of the eighteenth aspect, administration of the anti-TIGIT antagonist antibody and anti-PD-L1 antagonist antibody results in a clinical response. In some embodiments, the clinical response is an increase in the objective response rate (ORR) of the subject as compared to a reference ORR. In some embodiments, the reference ORR is the median ORR of a population of subjects who have received a treatment comprising an anti-PD-L1 antagonist antibody without an anti-TIGIT antagonist antibody. In some embodiments, the clinical response is an increase in the progression-free survival (PFS) of the subject as compared to a reference PFS time. In some embodiments, the reference PFS time is the median PFS time of a population of subjects who have received a treatment comprising an anti-PD-L1 antagonist antibody without an anti-TIGIT antagonist antibody.

In a nineteenth aspect, the invention features an anti-TIGIT antagonist antibody and atezolizumab for use in a method of treating a subject having a NSCLC, the method comprising administering to the subject one or more dosing cycles of an anti-TIGIT antagonist antibody at a fixed dose of 600 mg every three weeks and atezolizumab at a fixed dose of 1200 mg every three weeks, wherein the anti-TIGIT antagonist antibody comprises: a VH domain comprising the amino acid sequence of SEQ ID NO: 17 or 18 and a VL domain comprising the amino acid sequence of SEQ ID NO: 19.

In a twentieth aspect, the invention features tiragolumab and atezolizumab for use in a method of treating a subject having a NSCLC, the method comprising administering to the subject one or more dosing cycles of tiragolumab at a fixed dose of 600 mg every three weeks and atezolizumab at a fixed dose of 1200 mg every three weeks.

In a twenty-first aspect, the invention features a use of an anti-TIGIT antagonist antibody and an anti-PD-L1 antagonist antibody in the manufacture of a medicament for use in a method of treating a subject having a lung cancer, the method comprising administering to the subject one or more dosing cycles of the medicament, wherein the medicament is formulated for administration of the anti-TIGIT antagonist antibody at a fixed dose of between about 30 mg to about 1200 mg every three weeks and the anti-PD-L1 antagonist antibody at a fixed dose of between about 80 mg to about 1600 mg every three weeks.

In a twenty-second aspect, the invention features a use of an anti-TIGIT antagonist antibody in the manufacture of a medicament for use in a method of treating a subject having lung cancer, the method comprising administering to the subject one or more dosing cycles of the medicament and an anti-PD-L1 antagonist antibody, wherein the medicament is formulated for administration of the anti-TIGIT antagonist antibody at a fixed dose of between about 30 mg to about 1200 mg every three weeks and the anti-PD-L1 antagonist antibody is to be administered at a fixed dose of between about 80 mg to about 1600 mg every three weeks.

In an twenty-third aspect, the invention features a use of an anti-PD-L1 antagonist antibody in the manufacture of a medicament for use in a method of treating a subject having lung cancer, the method comprising administering to the subject one or more dosing cycles of the medicament and an anti-TIGIT antagonist antibody, wherein the medicament is formulated for administration of the anti-PD-L1 antagonist antibody at a fixed dose of between about 80 mg to about 1600 mg every three weeks and the anti-TIGIT antagonist antibody is to be administered at a fixed dose of between about 30 mg to about 1200 mg every three weeks.

In some embodiments of any of the twenty-first, twenty-second, and twenty-third aspects, the anti-TIGIT antagonist antibody is to be administered to the subject at a fixed dose of between about 30 mg to about 600 mg every three weeks. In some embodiments, the anti-TIGIT antagonist antibody is to be administered to the subject at a fixed dose of about 600 mg every three weeks.

In some embodiments of any of the twenty-first, twenty-second, and twenty-third aspects, the anti-TIGIT antagonist antibody comprises the following hypervariable regions (HVRs): an HVR-H1 sequence comprising the amino acid sequence of SNSAAWN (SEQ ID NO: 1); an HVR-H2 sequence comprising the amino acid sequence of KTYYRFKWYSDYAVSVKG (SEQ ID NO: 2); an HVR-H3 sequence comprising the amino acid sequence of ESTTYDLLAGPFDY (SEQ ID NO: 3); an HVR-L1 sequence comprising the amino acid sequence of KSSQTVLYSSNNKKYLA (SEQ ID NO: 4); an HVR-L2 sequence comprising the amino acid sequence of WASTRES (SEQ ID NO: 5); and an HVR-L3 sequence comprising the amino acid sequence of QQYYSTPFT (SEQ ID NO: 6). In some embodiments, the anti-TIGIT antagonist antibody further comprises the following light chain variable region framework regions (FRs): an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and an FR-L4 comprising the amino acid sequence of FGPGTKVEIK (SEQ ID NO: 10). In some embodiments, the anti-TIGIT antagonist antibody further comprises the following heavy chain variable region FRs: an FR-H1 comprising the amino acid sequence of X₁VQLQQSGPGLVKPSQTLSLTCAISGDSVS (SEQ ID NO: 11), wherein X₁ is Q or E; an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14). In some embodiments, X₁ is Q. In some embodiments, X₁ is E.

In some embodiments of any of the twenty-first, twenty-second, and twenty-third aspects, the anti-TIGIT antagonist antibody comprises: (a) a heavy chain variable (VH) domain comprising an amino acid sequence having at least 95% sequence identity to the amino acid sequence of SEQ ID NO: 17 or 18; (b) a light chain variable (VL) domain comprising an amino acid sequence having at least 95% sequence identity to the amino acid sequence of SEQ ID NO: 19; or (c) a VH domain as in (a) and a VL domain as in (b).

In some embodiments of any of the twenty-first, twenty-second, and twenty-third aspects, the anti-TIGIT antagonist antibody is a monoclonal antibody. In some embodiments, the anti-TIGIT antagonist antibody is a human antibody (e.g., a monoclonal human antibody).

In some embodiments of any of the twenty-first, twenty-second, and twenty-third aspects, the anti-TIGIT antagonist antibody is a full-length antibody. In some embodiments of any of the twenty-first, twenty-second, and twenty-third aspects, the anti-TIGIT antagonist antibody is tiragolumab.

In some embodiments of any of the twenty-first, twenty-second, and twenty-third aspects, the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

In some embodiments of any of the twenty-first, twenty-second, and twenty-third aspects, the anti-TIGIT antagonist antibody is an IgG class antibody. In some embodiments, the IgG class antibody is an IgG1 subclass antibody.

In some embodiments of any of the twenty-first, twenty-second, and twenty-third aspects, anti-PD-L1 antagonist antibody is to be administered to the subject at a fixed dose of about 1200 mg every three weeks.

In some embodiments of any of the twenty-first, twenty-second, and twenty-third aspects, the anti-PD-L1 antagonist antibody is atezolizumab (MPDL3280A), YW243.55.S70, MSB0010718C, MDX-1105, or MEDI4736. In some embodiments, the anti-PD-L1 antagonist antibody is atezolizumab.

In some embodiments of any of the twenty-first, twenty-second, and twenty-third aspects, the anti-PD-L1 antagonist antibody comprises the following HVRs: an HVR-H1 sequence comprising the amino acid sequence of GFTFSDSWIH (SEQ ID NO: 20); an HVR-H2 sequence comprising the amino acid sequence of AWISPYGGSTYYADSVKG (SEQ ID NO: 21); an HVR-H3 sequence comprising the amino acid sequence of RHWPGGFDY (SEQ ID NO: 22); an HVR-L1 sequence comprising the amino acid sequence of RASQDVSTAVA (SEQ ID NO: 23); an HVR-L2 sequence comprising the amino acid sequence of SASFLYS (SEQ ID NO: 24); and an HVR-L3 sequence comprising the amino acid sequence of QQYLYHPAT (SEQ ID NO: 25). In some embodiments, the anti-PD-L1 antagonist antibody comprises: (a) a heavy chain variable (VH) domain comprising an amino acid sequence having at least 95% sequence identity to the amino acid sequence of SEQ ID NO: 26; (b) a light chain variable (VL) domain comprising an amino acid sequence having at least 95% sequence identity to the amino acid sequence of SEQ ID NO: 27; or (c) a VH domain as in (a) and a VL domain as in (b). In some embodiments, the anti-PD-L1 antagonist antibody comprises: a VH domain comprising the amino acid sequence of SEQ ID NO: 26 and a VL domain comprising the amino acid sequence of SEQ ID NO: 27.

In some embodiments of any of the twenty-first, twenty-second, and twenty-third aspects, the anti-PD-L1 antagonist antibody is a monoclonal antibody. In some embodiments, the anti-PD-L1 antagonist antibody is a humanized antibody (e.g., a monoclonal humanized antibody).

In some embodiments of any of the twenty-first, twenty-second, and twenty-third aspects, the anti-PD-L1 antagonist antibody is a full-length antibody.

In some embodiments of any of the twenty-first, twenty-second, and twenty-third aspects, the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

In some embodiments of any of the twenty-first, twenty-second, and twenty-third aspects, the anti-PD-L1 antagonist antibody is an IgG class antibody. In some embodiments, the IgG class antibody is an IgG1 subclass antibody.

In some embodiments of any of the twenty-first, twenty-second, and twenty-third aspects, the anti-TIGIT antagonist antibody is to be administered to the subject at a fixed dose of about 600 mg of every three weeks and the anti-PD-L1 antagonist antibody is to be administered to the subject at a fixed dose of about 1200 mg every three weeks.

In some embodiments of any of the twenty-first, twenty-second, and twenty-third aspects, the length of each of the one or more dosing cycles is 21 days.

In some embodiments of any of the twenty-first, twenty-second, and twenty-third aspects, the anti-TIGIT antagonist antibody and anti-PD-L1 antagonist antibody are to be administered to the subject on about Day 1 of each of the one or more dosing cycles.

In some embodiments of any of the twenty-first, twenty-second, and twenty-third aspects, the anti-TIGIT antagonist antibody is to be administered to the subject before the anti-PD-L1 antagonist antibody. In some embodiments, a first observation period is to follow administration of the anti-TIGIT antagonist antibody and second observation period is to follow administration of the anti-PD-L1 antagonist antibody. In some embodiments, the first observation period and the second observation period are each between about 30 minutes to about 60 minutes in length.

In some embodiments of any of the twenty-first, twenty-second, and twenty-third aspects, the anti-PD-L1 antagonist antibody is to be administered to the subject before the anti-TIGIT antagonist antibody. In some embodiments, a first observation period is to follow administration of the anti-PD-L1 antagonist antibody and second observation period is to follow administration of the anti-TIGIT antagonist antibody. In some embodiments, the first observation period and the second observation period are each between about 30 minutes to about 60 minutes in length.

In some embodiments of the twenty-first aspect, the anti-TIGIT antagonist antibody is to be administered to the subject simultaneously with the anti-PD-L1 antagonist antibody.

In some embodiments of any of the twenty-first, twenty-second, and twenty-third aspects, the anti-TIGIT antagonist antibody and anti-PD-L1 antagonist antibody are to be administered to the subject intravenously. In some embodiments, the anti-TIGIT antagonist antibody is to be administered to the subject by intravenous infusion over 60±10 minutes. In some embodiments, the anti-PD-L1 antagonist antibody is to be administered to the subject by intravenous infusion over 60±15 minutes.

In some embodiments of any of the twenty-first, twenty-second, and twenty-third aspects, a tumor sample obtained from the subject has been determined to have a detectable expression level of PD-L1.

In some embodiments of any of the twenty-first, twenty-second, and twenty-third aspects, the detectable expression level of PD-L1 is a detectable protein expression level of PD-L1. In some embodiments, the detectable protein expression level of PD-L1 has been determined by an immunohistochemical (IHC) assay. In some embodiments, the IHC assay uses anti-PD-L1 antibody 22C3, SP142, SP263, or 28-8.

In some embodiments of any of the twenty-first, twenty-second, and twenty-third aspects, the IHC assay uses anti-PD-L1 antibody 22C3. In some embodiments, the tumor sample has been determined to have a tumor proportion score (TPS) of greater than, or equal to, 1%. In some embodiments, the TPS is greater than, or equal to, 1% and less than 50%. In some embodiments, the TPS is greater than, or equal to, 50%.

In some embodiments of any of the twenty-first, twenty-second, and twenty-third aspects, the IHC assay uses anti-PD-L1 antibody SP142. In some embodiments, the tumor sample has been determined to have a detectable expression level of PD-L1 in greater than, or equal to, 1% of the tumor cells in the tumor sample. In some embodiments, the tumor sample has been determined to have a detectable expression level of PD-L1 in greater than, or equal to, 1% and less than 5% of the tumor cells in the tumor sample. In some embodiments, the tumor sample has been determined to have a detectable expression level of PD-L1 in greater than, or equal to, 5% and less than 50% of the tumor cells in the tumor sample. In some embodiments, the tumor sample has been determined to have a detectable expression level of PD-L1 in greater than, or equal to, 50% of the tumor cells in the tumor sample. In some embodiments, the tumor sample has been determined to have a detectable expression level of PD-L1 in tumor-infiltrating immune cells that comprise greater than, or equal to, 1% of the tumor sample. In some embodiments, the tumor sample has been determined to have a detectable expression level of PD-L1 in tumor-infiltrating immune cells that comprise greater than, or equal to, 1% and less than 5% of the tumor sample. In some embodiments, the tumor sample has been determined to have a detectable expression level of PD-L1 in tumor-infiltrating immune cells that comprise greater than, or equal to, 5% and less than 10% of the tumor sample. In some embodiments, the tumor sample has been determined to have a detectable expression level of PD-L1 in tumor-infiltrating immune cells that comprise greater than, or equal to, 10% of the tumor sample.

In some embodiments of any of the twenty-first, twenty-second, and twenty-third aspects, the detectable expression level of PD-L1 is a detectable nucleic acid expression level of PD-L1. In some embodiments, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

In some embodiments of any of the twenty-first, twenty-second, and twenty-third aspects, the lung cancer is a non-small cell lung cancer (NSCLC).

In some embodiments of any of the nineteenth, twentieth, twenty-first, twenty-second, and twenty-third aspects, the NSCLC is a squamous NSCLC. In some embodiments, the NSCLC is a non-squamous NSCLC. In some embodiments, the NSCLC is a locally advanced unresectable NSCLC. In some embodiments, the NSCLC is a Stage IIIB NSCLC. In some embodiments, the NSCLC is a recurrent or metastatic NSCLC. In some embodiments, the NSCLC is a Stage IV NSCLC. In some embodiments, the subject has not been previously treated for Stage IV NSCLC.

In some embodiments of any of the nineteenth, twentieth, twenty-first, twenty-second, and twenty-third aspects, the subject does not have a sensitizing epidermal growth factor receptor (EGFR) gene mutation or anaplastic lymphoma kinase (ALK) gene rearrangement.

In some embodiments of any of the nineteenth, twentieth, twenty-first, twenty-second, and twenty-third aspects, the subject does not have a pulmonary lymphoepithelioma-like carcinoma subtype of NSCLC.

In some embodiments of any of the nineteenth, twentieth, twenty-first, twenty-second, and twenty-third aspects, the subject does not have an active EBV infection or a known or suspected chronic active EBV infection. In some embodiments, the subject is negative for EBV IgM or negative by EBV PCR. In some embodiments, the subject is negative for EBV IgM and negative by EBV PCR. In some embodiments, the subject is positive for EBV IgG or positive for EBNA. In some embodiments, the subject is positive for EBV IgG and positive for EBNA.

In some embodiments of any of the nineteenth, twentieth, twenty-first, twenty-second, and twenty-third aspects, the subject is negative for EBV IgG or negative for EBNA. In some embodiments, the subject is negative for EBV IgG and negative for EBNA.

In some embodiments of any of the twenty-first, twenty-second, and twenty-third aspects, administration of the anti-TIGIT antagonist antibody and anti-PD-L1 antagonist antibody results in a clinical response. In some embodiments, the clinical response is an increase in the objective response rate (ORR) of the subject as compared to a reference ORR. In some embodiments, the reference ORR is the median ORR of a population of subjects who have received a treatment comprising an anti-PD-L1 antagonist antibody without an anti-TIGIT antagonist antibody. In some embodiments, the clinical response is an increase in the progression-free survival (PFS) of the subject as compared to a reference PFS time. In some embodiments, the reference PFS time is the median PFS time of a population of subjects who have received a treatment comprising an anti-PD-L1 antagonist antibody without an anti-TIGIT antagonist antibody.

In a twenty-fourth aspect, the invention features a use of an anti-TIGIT antagonist antibody and atezolizumab in the manufacture of a medicament for use in a method of treating a subject having a NSCLC, the method comprising administering to the subject one or more dosing cycles of the medicament, wherein the medicament is formulated for administration of the anti-TIGIT antagonist antibody at a fixed dose of 600 mg every three weeks and atezolizumab at a fixed dose of 1200 mg every three weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19.

In a twenty-fifth aspect, the invention features a use of an anti-TIGIT antagonist antibody in the manufacture of a medicament for use in a method of treating a subject having a NSCLC, the method comprising administering to the subject one or more dosing cycles of the medicament and atezolizumab, wherein the medicament is formulated for administration of the anti-TIGIT antagonist antibody at a fixed dose of 600 mg every three weeks and atezolizumab is to be administered at a fixed dose of 1200 mg every three weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19.

In a twenty-sixth aspect, the invention features a use of atezolizumab in the manufacture of a medicament for use in a method of treating a subject having a NSCLC, the method comprising administering to the subject one or more dosing cycles of the medicament and an anti-TIGIT antagonist antibody, wherein the medicament is formulated for administration of atezolizumab at a fixed dose of 1200 mg every three weeks and the anti-TIGIT antagonist antibody is to be administered at a fixed dose of 600 mg every three weeks, and wherein the anti-TIGIT antagonist antibody comprises: a VH domain comprising the amino acid sequence of SEQ ID NO: 17 or 18 and a VL domain comprising the amino acid sequence of SEQ ID NO: 19.

In a twenty-seventh aspect, the invention features a use of tiragolumab and atezolizumab in the manufacture of a medicament for use in a method of treating a subject having a NSCLC, the method comprising administering to the subject one or more dosing cycles of the medicament, wherein the medicament is formulated for administration of tiragolumab at a fixed dose of 600 mg every three weeks and atezolizumab at a fixed dose of 1200 mg every three weeks.

In a twenty-eighth aspect, the invention features a use of tiragolumab in the manufacture of a medicament for use in a method of treating a subject having a NSCLC, the method comprising administering to the subject one or more dosing cycles of the medicament and atezolizumab, wherein the medicament is formulated for administration of tiragolumab at a fixed dose of 600 mg every three weeks and atezolizumab is to be administered at a fixed dose of 1200 mg every three weeks.

In a twenty-ninth aspect, the invention features a use of atezolizumab in the manufacture of a medicament for use in a method of treating a subject having a NSCLC, the method comprising administering to the subject one or more dosing cycles of the medicament and tiragolumab, wherein the medicament is formulated for administration of atezolizumab at a fixed dose of 1200 mg every three weeks and tiragolumab is to be administered at a fixed dose of 600 mg every three weeks.

In some embodiments of any of the twenty-fourth, twenty-fifth, twenty-sixth, twenty-seventh, and twenty-eighth, and twenty-ninth aspects, the subject does not have a pulmonary lymphoepithelioma-like carcinoma subtype of NSCLC.

In some embodiments of any of the twenty-fourth, twenty-fifth, twenty-sixth, twenty-seventh, twenty-eighth, and twenty-ninth aspects, the subject does not have a sensitizing epidermal growth factor receptor (EGFR) gene mutation or anaplastic lymphoma kinase (ALK) gene rearrangement.

In some embodiments of any of the twenty-fourth, twenty-fifth, twenty-sixth, twenty-seventh, twenty-eighth, and twenty-ninth aspects, the subject does not have an active EBV infection or a known or suspected chronic active EBV infection. In some embodiments, the subject is negative for EBV IgM or negative by EBV PCR. In some embodiments, the subject is negative for EBV IgM and negative by EBV PCR. In some embodiments, the subject is positive for EBV IgG or positive for EBNA. In some embodiments, the subject is positive for EBV IgG and positive for EBNA.

In some embodiments of any of the twenty-fourth, twenty-fifth, twenty-sixth, twenty-seventh, twenty-eighth, and twenty-ninth aspects, the subject is negative for EBV IgG or negative for EBNA. In some embodiments, the subject is negative for EBV IgG and negative for EBNA.

In a thirtieth aspect, the invention features a kit comprising an anti-TIGIT antagonist antibody, an anti-PD-L1 antagonist antibody, and a package insert comprising instructions to administer the anti-TIGIT antagonist antibody and the anti-PD-L1 antagonist antibody to a subject having a lung cancer in accordance with the methods of any one of the embodiments of any of the first, second, third, fourth, seventh, eighth, and ninth aspects.

### DETAILED DESCRIPTION OF THE INVENTION

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X.”

The “amount,” “level,” or “expression level,” used herein interchangeably, of a biomarker is a detectable level in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs). Expression levels can be measured by methods known to one skilled in the art and also disclosed herein. The expression level or amount of a biomarker (e.g., PD-L1) can be used to identify/characterize a subject having a cancer (e.g., lung cancer, e.g., NSCLC, e.g., squamous or non-squamous NSCLC, e.g., locally advanced unresectable NSCLC (e.g., Stage IIIB NSCLC), or recurrent or metastatic NSCLC (e.g., Stage IV NSCLC)) who may be likely to respond to, or benefit from, a particular therapy (e.g., a therapy comprising one or more dosing cycles of an anti-TIGIT antagonist antibody and an anti-PD-L1 antagonist antibody).

The presence and/or expression level/amount of various biomarkers described herein in a sample can be analyzed by a number of methodologies, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), MassARRAY, proteomics, quantitative blood based assays (e.g., Serum ELISA), biochemical enzymatic activity assays, in situ hybridization, fluorescence in situ hybridization (FISH), Southern analysis, Northern analysis, whole genome sequencing, massively parallel DNA sequencing (e.g., next-generation sequencing), NANOSTRING®, polymerase chain reaction (PCR) including quantitative real time PCR (qRT-PCR) and other amplification type detection methods, such as, for example, branched DNA, SISBA, TMA and the like, RNA-seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

The term “TIGIT” or “T-cell immunoreceptor with Ig and ITIM domains” as used herein refers to any native TIGIT from any vertebrate source, including mammals such as primates (e.g., humans) and rodents (e.g., mice and rats), unless otherwise indicated. TIGIT is also known in the art as DKFZp667A205, FLJ39873, V-set and immunoglobulin domain-containing protein 9, V-set and transmembrane domain-containing protein 3, VSIG9, VSTM3, and WUCAM. The term encompasses “full-length,” unprocessed TIGIT (e.g., full-length human TIGIT having the amino acid sequence of SEQ ID NO: 30), as well as any form of TIGIT that results from processing in the cell (e.g., processed human TIGIT without a signal sequence, having the amino acid sequence of SEQ ID NO: 31). The term also encompasses naturally occurring variants of TIGIT, e.g., splice variants or allelic variants. The amino acid sequence of an exemplary human TIGIT may be found under UniProt Accession Number Q495A1.

The term “PD-L1” or “Programmed Cell Death Ligand 1” refers herein to any native PD-L1 from any vertebrate source, including mammals such as primates (e.g., humans) and rodents (e.g., mice and rats), unless otherwise indicated. PD-L1 is also known in the art as CD274 molecule, CD274 antigen, B7 homolog 1, PDCD1 Ligand 1, PDCD1 LG1, PDCD1 L1, B7H1, PDL1, programmed death ligand 1, B7-H1, and B7-H. The term also encompasses naturally occurring variants of PD-L1, e.g., splice variants, or allelic variants. The amino acid sequence of an exemplary human PD-L1 may be found under UniProt Accession Number Q9NZQ7 (SEQ ID NO: 32).

The term “antagonist” is used in the broadest sense, and includes any molecule that partially or fully blocks, inhibits, or neutralizes a biological activity of a native polypeptide disclosed herein. Suitable antagonist molecules specifically include antagonist antibodies or antibody fragments (e.g., antigen-binding fragments), fragments or amino acid sequence variants of native polypeptides, peptides, antisense oligonucleotides, small organic molecules, etc. Methods for identifying antagonists of a polypeptide may comprise contacting a polypeptide with a candidate antagonist molecule and measuring a detectable change in one or more biological activities normally associated with the polypeptide.

The term “anti-TIGIT antagonist antibody” refers to an antibody or an antigen-binding fragment or variant thereof that is capable of binding TIGIT with sufficient affinity such that it substantially or completely inhibits the biological activity of TIGIT. For example, an anti-TIGIT antagonist antibody may block signaling through PVR, PVRL2, and/or PVRL3 so as to restore a functional response by T-cells (e.g., proliferation, cytokine production, target cell killing) from a dysfunctional state to antigen stimulation. It will be understood by one of ordinary skill in the art that in some instances, an anti-TIGIT antagonist antibody may antagonize one TIGIT activity without affecting another TIGIT activity. For example, an anti-TIGIT antagonist antibody for use in certain of the methods or uses described herein is an anti-TIGIT antagonist antibody that antagonizes TIGIT activity in response to one of PVR interaction, PVRL3 interaction, or PVRL2 interaction, e.g., without affecting or minimally affecting any of the other TIGIT interactions. In one embodiment, the extent of binding of an anti-TIGIT antagonist antibody to an unrelated, non-TIGIT protein is less than about 10% of the binding of the antibody to TIGIT as measured, e.g., by a radioimmunoassay (RIA). In certain embodiments, an anti-TIGIT antagonist antibody that binds to TIGIT has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g. from 10⁻⁸ M to 10⁻¹³ M, e.g., from 10⁻⁹M to 10⁻¹³ M). In certain embodiments, an anti-TIGIT antagonist antibody binds to an epitope of TIGIT that is conserved among TIGIT from different species or an epitope on TIGIT that allows for cross-species reactivity. In one embodiment, the anti-TIGIT antagonist antibody is tiragolumab.

The term “anti-PD-L1 antagonist antibody” refers to an antibody or antigen-binding fragment or variant thereof that is capable of binding PD-L1 with sufficient affinity such that it substantially or completely inhibits the biological activity of PD-L1 (e.g., abrogates or interferes with signal transduction resulting from the interaction of PD-L1 with either one or more of its binding partners, such as PD-1, B7-1). For example, an anti-PD-L1 antagonist antibody may reduce the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, an anti-PD-L1 antagonist antibody is a molecule that inhibits the binding of PD-L1 to its binding partners. In a specific aspect, the anti-PD-L1 antagonist antibody inhibits binding of PD-L1 to PD-1 and/or B7-1. In one embodiment, the extent of binding of an anti-PD-L1 antagonist antibody to an unrelated, non-PD-L1 protein is less than about 10% of the binding of the antibody to PD-L1 as measured, e.g., by a radioimmunoassay (RIA). In certain embodiments, an anti-PD-L1 antagonist antibody that binds to PD-L1 has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g. from 10⁻⁸M to 10⁻¹³M, e.g., from 10⁻⁹M to 10⁻¹³ M). In certain embodiments, an anti-PD-L1 antagonist antibody binds to an epitope of PD-L1 that is conserved among PD-L1 from different species. In some embodiments, the anti-PD-L1 antagonist antibody is MPDL3280A (atezolizumab), MDX-1105, MEDI4736 (durvalumab), YW243.55.S70, or MSB0010718C (avelumab). In a specific aspect, an anti-PD-L1 antagonist antibody is atezolizumab.

As used herein, “administering” is meant a method of giving a dosage of a compound (e.g., an anti-TIGIT antagonist antibody or an anti-PD-L1 antagonist antibody) or a composition (e.g., a pharmaceutical composition, e.g., a pharmaceutical composition including an anti-TIGIT antibody and/or anti-PD-L1 antibody) to a subject. The compounds and/or compositions utilized in the methods described herein can be administered, for example, intravenously (e.g., by intravenous infusion), subcutaneously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated).

A “fixed” or “flat” dose of a therapeutic agent (e.g., an anti-TIGIT antagonist antibody and/or an anti-PD-L1 antagonist antibody) herein refers to a dose that is administered to a patient without regard for the weight or body surface area (BSA) of the patient. The fixed or flat dose is therefore not provided as a mg/kg dose or a mg/m² dose, but rather as an absolute amount of the therapeutic agent (e.g., mg).

As used herein, the term “treatment” or “treating” refers to clinical intervention designed to alter the natural course of the individual or cell being treated during the course of clinical pathology. Desirable effects of treatment include delaying or decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “in conjunction with” refers to administration of one treatment modality in addition to another treatment modality. As such, “in conjunction with” refers to administration of one treatment modality before, during, or after administration of the other treatment modality to the individual.

A “disorder” or “disease” is any condition that would benefit from treatment including, but not limited to, disorders that are associated with some degree of abnormal cell proliferation, e.g., cancer, e.g., lung cancer, e.g., NSCLC, e.g., squamous or non-squamous NSCLC, e.g., locally advanced unresectable NSCLC (e.g., Stage IIIB NSCLC), or recurrent or metastatic NSCLC (e.g., Stage IV NSCLC)).

The term “dysfunction,” in the context of immune dysfunction, refers to a state of reduced immune responsiveness to antigenic stimulation.

The term “dysfunctional,” as used herein, also includes refractory or unresponsive to antigen recognition, specifically, impaired capacity to translate antigen recognition into downstream T-cell effector functions, such as proliferation, cytokine production (e.g., gamma interferon) and/or target cell killing.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include, but are not limited to, lung cancer, such as non-small cell lung cancer (NSCLC), which includes squamous NSCLC or non-squamous NSCLC, including locally advanced unresectable NSCLC (e.g., Stage IIIB NSCLC), or recurrent or metastatic NSCLC (e.g., Stage IV NSCLC), adenocarcinoma of the lung, or squamous cell cancer (e.g., epithelial squamous cell cancer); esophageal cancer; cancer of the peritoneum; hepatocellular cancer; gastric or stomach cancer, including gastrointestinal cancer and gastrointestinal stromal cancer; pancreatic cancer; glioblastoma; cervical cancer; ovarian cancer; liver cancer; bladder cancer (e.g., urothelial bladder cancer (UBC), muscle invasive bladder cancer (MIBC), and BCG-refractory non-muscle invasive bladder cancer (NMIBC)); cancer of the urinary tract; hepatoma; breast cancer (e.g., HER2+ breast cancer and triple-negative breast cancer (TNBC), which are estrogen receptors (ER−), progesterone receptors (PR−), and HER2 (HER2−) negative); colon cancer; rectal cancer; colorectal cancer; endometrial or uterine carcinoma; salivary gland carcinoma; kidney or renal cancer (e.g., renal cell carcinoma (RCC)); prostate cancer; vulval cancer; thyroid cancer; hepatic carcinoma; anal carcinoma; penile carcinoma; melanoma, including superficial spreading melanoma, lentigo maligna melanoma, acral lentiginous melanomas, and nodular melanomas; multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); acute myologenous leukemia (AML); hairy cell leukemia; chronic myeloblastic leukemia (CML); post-transplant lymphoproliferative disorder (PTLD); and myelodysplastic syndromes (MDS), as well as abnormal vascular proliferation associated with phakomatoses, edema (such as that associated with brain tumors), Meigs' syndrome, brain cancer, head and neck cancer, and associated metastases.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage, and tumor clearance.

As used herein, “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

The term “anti-cancer therapy” refers to a therapy useful in treating cancer (e.g., lung cancer, e.g., NSCLC, e.g., squamous or non-squamous NSCLC, e.g., locally advanced unresectable NSCLC (e.g., Stage IIIB NSCLC), or recurrent or metastatic NSCLC (e.g., Stage IV NSCLC)). Examples of anti-cancer therapeutic agents include, but are limited to, e.g., immunomodulatory agents (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), an anti-TIGIT antagonist antibody, or an anti-PD-L1 antagonist antibody, or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), chemotherapeutic agents, growth inhibitory agents, cytotoxic agents, agents used in radiation therapy, anti-angiogenesis agents, apoptotic agents, anti-tubulin agents, and other agents to treat cancer. Combinations thereof are also included in the invention.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anti-cancer agents disclosed below.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethyl hydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors (e.g., an anaplastic lymphoma kinase (Alk) inhibitor, such as AF-802 (also known as CH-5424802 or alectinib)); (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; inhibitors of insulin receptor tyrosine kinases, including anaplastic lymphoma kinase (Alk) inhibitors, such as AF-802 (also known as CH-5424802 or alectinib), ASP3026, X396, LDK378, AP26113, crizotinib (XALKORI®), and ceritinib (ZYKADIA®); small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At211, I131, I125, Y90, Re186, Re188, Sm153, Bi212, P32, Pb212 and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

An “effective amount” of a compound, for example, an anti-TIGIT antagonist antibody or anti-PD-L1 antagonist antibody, or a composition (e.g., pharmaceutical composition) thereof, is at least the minimum amount required to achieve the desired therapeutic result, such as a measurable increase in overall survival or progression-free survival of a particular disease or disorder (e.g., cancer, e.g., lung cancer, e.g., NSCLC, e.g., squamous or non-squamous NSCLC, e.g., locally advanced unresectable NSCLC (e.g., Stage IIIB NSCLC), or recurrent or metastatic NSCLC (e.g., Stage IV NSCLC)). An effective amount herein may vary according to factors such as the disease state, age, sex, and weight of the patient, and the ability of the antibody to elicit a desired response in the subject. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications, and intermediate pathological phenotypes presenting during development of the disease. For therapeutic use, beneficial or desired results include clinical results such as decreasing one or more symptoms resulting from the disease (e.g., reduction or delay in cancer-related pain, symptomatic skeletal-related events (SSE), reduction in symptoms per the European Organization for Research and Treatment of Cancer Quality-of-Life Questionnaire (EORTC QLQ-C30, e.g., fatigue, nausea, vomiting, pain, dyspnea, insomnia, appetite loss, constipation, diarrhea, or general level of physical emotional, cognitive, or social functioning), reduction in pain as measured by, e.g., the 10-point pain severity (measured at its worst) numerical rating scale (NRS), and/or reduction in symptoms associated with lung cancer per the health-related quality of life (HRQoL) questionnaire as assessed by symptoms in lung cancer (SILC) scale (e.g., time to deterioration (TTD) in cough dyspenea and chest pain), increasing the quality of life of those suffering from the disease, decreasing the dose of other medications required to treat the disease, enhancing effect of another medication such as via targeting, delaying the progression of the disease (e.g. progression-free survival or radiographic progression-free survival (rPFS); delay of unequivocal clinical progression (e.g., cancer-related pain progression, symptomatic skeletal-related event, deterioration in Eastern Cooperative Group Oncology Group (ECOG) Performance Status (PS) (e.g., how the disease affects the daily living abilities of the patient), and/or initiation of next systemic anti-cancer therapy), and/or delaying time to lung-specific antigen progression), and/or prolonging survival. In the case of cancer or tumor, an effective amount of the drug may have the effect in reducing the number of cancer cells; reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

“Immunogenicity” refers to the ability of a particular substance to provoke an immune response. Tumors are immunogenic and enhancing tumor immunogenicity aids in the clearance of the tumor cells by the immune response. Examples of enhancing tumor immunogenicity include but are not limited to treatment with a TIGIT and/or PD-L1 antagonist (e.g., anti-TIGIT antagonist antibodies and/or anti-PDL-1 antagonist antibodies).

“Individual response” or “response” can be assessed using any endpoint indicating a benefit to the subject, including, without limitation, (1) inhibition, to some extent, of disease progression (e.g., progression of cancer, e.g., lung cancer, e.g., NSCLC, e.g., squamous or non-squamous NSCLC, e.g., locally advanced unresectable NSCLC (e.g., Stage IIIB NSCLC), or recurrent or metastatic NSCLC (e.g., Stage IV NSCLC)), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e. reduction, slowing down or complete stopping) of metastasis; (5) relief, to some extent, of one or more symptoms associated with the disease or disorder (e.g., cancer, e.g., lung cancer, e.g., NSCLC, e.g., squamous or non-squamous NSCLC, e.g., locally advanced unresectable NSCLC (e.g., Stage IIIB NSCLC), or recurrent or metastatic NSCLC (e.g., Stage IV NSCLC)); (6) increase or extend in the length of survival, including overall survival and progression-free survival; and/or (9) decreased mortality at a given point of time following treatment.

As used herein, “complete response” or “CR” refers to disappearance of all target lesions.

As used herein, “partial response” or “PR” refers to at least a 30% decrease in the sum of the longest diameters (SLD) of target lesions, taking as reference the baseline SLD.

As used herein, “objective response rate” (ORR) refers to the sum of complete response (CR) rate and partial response (PR) rate.

As used herein, “duration of objective response” (DOR) is defined as the time from the first occurrence of a documented objective response to disease progression, or death from any cause within 30 days of the last dose of a treatment, whichever occurs first.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0×length of the treatment duration.

As used herein, “survival” refers to the patient remaining alive, and includes overall survival as well as progression-free survival.

As used herein, “overall survival” (OS) refers to the percentage of subjects in a group who are alive after a particular duration of time, e.g., 1 year or 5 years from the time of diagnosis or treatment.

As used herein, “progression-free survival” (PFS) refers to the length of time during and after treatment during which the disease being treated (e.g., cancer, e.g., lung cancer, e.g., NSCLC, e.g., squamous or non-squamous NSCLC, e.g., locally advanced unresectable NSCLC (e.g., Stage IIIB NSCLC), or recurrent or metastatic NSCLC (e.g., Stage IV NSCLC)) does not get worse. Progression-free survival may include the amount of time patients have experienced a complete response or a partial response, as well as the amount of time patients have experienced stable disease.

As used herein, “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “delaying progression” of a disorder or disease means to defer, hinder, slow, retard, stabilize, and/or postpone development of the disease or disorder (e.g., cancer, e.g., lung cancer, e.g., NSCLC, e.g., squamous or non-squamous NSCLC, e.g., locally advanced unresectable NSCLC (e.g., Stage IIIB NSCLC), or recurrent or metastatic NSCLC (e.g., Stage IV NSCLC)). This delay can be of varying lengths of time, depending on the history of the disease and/or subject being treated. As is evident to one skilled in the art, a sufficient or significant delay can, in effect, encompass prevention, in that the subject does not develop the disease. For example, in a late stage cancer, development of central nervous system (CNS) metastasis, may be delayed.

As used herein, the term “reducing or inhibiting cancer relapse” means to reduce or inhibit tumor or cancer relapse, or tumor or cancer progression.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated (e.g., cancer, e.g., lung cancer, e.g., NSCLC, e.g., squamous or non-squamous NSCLC, e.g., locally advanced unresectable NSCLC (e.g., Stage IIIB NSCLC), or recurrent or metastatic NSCLC (e.g., Stage IV NSCLC)), the presence or size of metastases, or the size of the primary tumor.

By “extending survival” is meant increasing overall or progression free survival in a treated patient relative to an untreated patient (e.g., relative to a patient not treated with the medicament), or relative to a patient who does not express a biomarker at the designated level, and/or relative to a patient treated with an approved anti-tumor agent. An objective response refers to a measurable response, including complete response (CR) or partial response (PR).

The terms “detecting” and “detection” are used herein in the broadest sense to include both qualitative and quantitative measurements of a target molecule. Detecting includes identifying the mere presence of the target molecule in a sample as well as determining whether the target molecule is present in the sample at detectable levels. Detecting may be direct or indirect.

As used herein, “tumor proportion score” (TPS) is the percentage of viable tumor cells showing partial or complete membrane staining (exclusive of cytoplasmic staining) at any intensity relative to all viable tumor cells present in a sample, following staining of the sample in the context of an immunohistochemical (IHC) assay, e.g., an IHC assay staining for PD-L1 using the antibody 22C3. Accordingly, a TPS may be calculated using the PD-L1 IHC 22C3 pharmDx assay (Dako), for example, by the formula TPS=(number of PD-L1-positive tumor cells)/(total number of PD-L1-positive and PD-L1 negative tumor cells), wherein PD-L1 cytoplasmic staining of tumor cells and all non-tumor cells (e.g., tumor-infiltrating immune cells, normal cells, necrotic cells, and debris) are excluded from evaluation and scoring.

A “tumor-infiltrating immune cell,” as used herein, refers to any immune cell present in a tumor or a sample thereof. Tumor-infiltrating immune cells include, but are not limited to, intratumoral immune cells, peritumoral immune cells, other tumor stroma cells (e.g., fibroblasts), or any combination thereof. Such tumor-infiltrating immune cells can be, for example, T lymphocytes (such as CD8+ T lymphocytes and/or CD4+ T lymphocytes), B lymphocytes, or other bone marrow-lineage cells, including granulocytes (e.g., neutrophils, eosinophils, and basophils), monocytes, macrophages, dendritic cells (e.g., interdigitating dendritic cells), histiocytes, and natural killer cells.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer, e.g., lung cancer, e.g., NSCLC, e.g., squamous or non-squamous NSCLC, e.g., locally advanced unresectable NSCLC (e.g., Stage IIIB NSCLC), or recurrent or metastatic NSCLC (e.g., Stage IV NSCLC)) characterized by certain, molecular, pathological, histological, and/or clinical features. In some embodiments, a biomarker is a gene. Biomarkers include, but are not limited to, polypeptides, polynucleotides (e.g., DNA, and/or RNA), polynucleotide copy number alterations (e.g., DNA copy numbers), polypeptide and polynucleotide modifications (e.g., posttranslational modifications), carbohydrates, and/or glycolipid-based molecular markers. In some embodiments, the biomarker is PD-L1.

The term “antibody” includes monoclonal antibodies (including full-length antibodies which have an immunoglobulin Fc region), antibody compositions with polyepitopic specificity, multispecific antibodies (e.g., bispecific antibodies), diabodies, and single-chain molecules, as well as antibody fragments, including antigen-binding fragments, such as Fab, F(ab′)₂, and Fv. The term “immunoglobulin” (Ig) is used interchangeably with “antibody” herein.

The basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6. The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ, and μ, respectively. The γ and α classes are further divided into subclasses on the basis of relatively minor differences in the CH sequence and function, e.g., humans express the following subclasses: IgG1, IgG2A, IgG2B, IgG3, IgG4, IgA1 and IgA2.

The term “hypervariable region” or “HVR” refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk, J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

The expression “variable-domain residue-numbering as in Kabat” or “amino-acid-position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy-chain variable domain may include a single amino acid insert (residue 52a according to Kabat) after residue 52 of H2 and inserted residues (e.g. residues 82a, 82b, and 82c, etc. according to Kabat) after heavy-chain FR residue 82. The Kabat numbering of residues may be determined for a given antibody by alignment at regions of homology of the sequence of the antibody with a “standard” Kabat numbered sequence.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, Md. (1991)). The constant domains are not involved directly in the binding of antibody to an antigen, but exhibit various effector functions, such as participation of the antibody in antibody-dependent cellular toxicity.

The “variable region” or “variable domain” of an antibody refers to the amino-terminal domains of the heavy or light chain of the antibody. The variable domains of the heavy chain and light chain may be referred to as “VH” and “VL”, respectively. These domains are generally the most variable parts of the antibody (relative to other antibodies of the same class) and contain the antigen binding sites.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full-length antibody,” “intact antibody,” and “whole antibody” are used interchangeably to refer to an antibody in its substantially intact form, as opposed to an antibody fragment. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen-binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Functional fragments” of the antibodies of the invention comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgG1, IgG2 (IgG2A, IgG2B), IgG3 and IgG4. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is used a subset of “chimeric antibodies.”

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

“Affinity” refers to the strength of the sum total of non-covalent interactions between a single binding site of a molecule (e.g., an antibody) and its binding partner (e.g., an antigen, e.g., TIGIT or PD-L1). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary embodiments for measuring binding affinity are described in the following.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see M. Daëron, Annu. Rev. Immunol. 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein.

A “human antibody” is an antibody that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

“Humanized” forms of non-human (e.g., murine) antibodies are chimeric antibodies that contain minimal sequence derived from non-human immunoglobulin. In one embodiment, a humanized antibody is a human immunoglobulin (recipient antibody) in which residues from an HVR (hereinafter defined) of the recipient are replaced by residues from an HVR of a non-human species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

The term an “isolated antibody” when used to describe the various antibodies disclosed herein, means an antibody that has been identified and separated and/or recovered from a cell or cell culture from which it was expressed. Contaminant components of its natural environment are materials that would typically interfere with diagnostic or therapeutic uses for the polypeptide, and can include enzymes, hormones, and other proteinaceous or non-proteinaceous solutes. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For a review of methods for assessment of antibody purity, see, e.g., Flatman et al., J. Chromatogr. B 848:79-87 (2007). In preferred embodiments, the antibody will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated antibody includes antibodies in situ within recombinant cells, because at least one component of the polypeptide natural environment will not be present. Ordinarily, however, isolated polypeptide will be prepared by at least one purification step.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (K_{D}) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. The term as used herein can be exhibited, for example, by a molecule having a K_{D} for the target of 10⁻⁴M or lower, alternatively 10⁻⁵M or lower, alternatively 10⁻⁶ M or lower, alternatively 10⁻⁷ M or lower, alternatively 10⁻⁸ M or lower, alternatively 10⁻⁹ M or lower, alternatively 10⁻¹⁰ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one embodiment, the term “specific binding” refers to binding where a molecule binds to a particular polypeptide or epitope on a particular polypeptide without substantially binding to any other polypeptide or polypeptide epitope.

The phrase “substantially reduced” or “substantially different,” as used herein, denotes a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., K_{D} values). The difference between said two values is, for example, greater than about 10%, greater than about 20%, greater than about 30%, greater than about 40%, and/or greater than about 50% as a function of the value for the reference/comparator molecule.

The term “substantially similar” or “substantially the same,” as used herein, denotes a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., K_{D} values). The difference between said two values is, for example, less than about 50%, less than about 40%, less than about 30%, less than about 20%, and/or less than about 10% as a function of the reference/comparator value.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “subject” or “individual” is meant a mammal, including, but not limited to, a human or non-human mammal, such as a bovine, equine, canine, ovine, or feline. In some embodiments, the subject is a human. Patients are also subjects herein.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “tumor sample,” “disease sample,” and variations thereof refers to any sample obtained from a subject of interest that would be expected or is known to contain the cellular and/or molecular entity that is to be characterized. In some embodiments, the sample is a tumor tissue sample (e.g., a lung cancer tumor tissue sample, e.g., an NSCLC tumor tissue sample, e.g., squamous or non-squamous NSCLC tumor tissue sample, e.g., locally advanced unresectable NSCLC tumor tissue sample (e.g., Stage IIIB NSCLC tumor tissue sample), or recurrent or metastatic NSCLC tumor tissue sample (e.g., Stage IV NSCLC tumor tissue sample). Other samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, stool, tumor lysates, and tissue culture medium, tissue extracts such as homogenized tissue, cellular extracts, and combinations thereof.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject. In yet another embodiment, a reference sample, reference cell, reference tissue, control sample, control cell, or control tissue is obtained from a healthy and/or non-diseased part of the body (e.g., tissues or cells) of a subject who is not the subject. In even another embodiment, a reference sample, reference cell, reference tissue, control sample, control cell, or control tissue is obtained from an untreated tissue and/or cell of the body of an individual who is not the subject.

The term “protein,” as used herein, refers to any native protein from any vertebrate source, including mammals such as primates (e.g., humans) and rodents (e.g., mice and rats), unless otherwise indicated. The term encompasses “full-length,” unprocessed protein as well as any form of the protein that results from processing in the cell. The term also encompasses naturally occurring variants of the protein, e.g., splice variants or allelic variants.

“Polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The terms “polynucleotide” and “nucleic acid” specifically includes mRNA and cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro-, or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The phrase “pharmaceutically acceptable” indicates that the substance or composition must be compatible chemically and/or toxicologically, with the other ingredients comprising a formulation, and/or the mammal being treated therewith.

The term “pharmaceutical formulation” refers to a preparation which is in such form as to permit the biological activity of an active ingredient contained therein to be effective, and which contains no additional components which are unacceptably toxic to a subject to which the formulation would be administered.

An “article of manufacture” is any manufacture (e.g., a package or container) or kit comprising at least one reagent, e.g., a medicament for treatment of a disease or disorder (e.g., cancer, e.g., lung cancer, e.g., NSCLC, e.g., squamous or non-squamous NSCLC, e.g., locally advanced unresectable NSCLC (e.g., Stage IIIB NSCLC), or recurrent or metastatic NSCLC (e.g., Stage IV NSCLC)), and a package insert. In certain embodiments, the manufacture or kit is promoted, distributed, or sold as a unit for performing the methods described herein.

A “package insert” refers to instructions customarily included in commercial packages of medicaments that contain information about the indications customarily included in commercial packages of medicaments that contain information about the indications, usage, dosage, administration, contraindications, other medicaments to be combined with the packaged product, and/or warnings concerning the use of such medicaments.

### III. Therapeutic Methods and Uses

Provided herein are methods and uses for treating cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) in a subject comprising administering to the subject one or more dosing cycles of an effective amount of an anti-TIGIT antagonist antibody and anti-PD-L1 antagonist antibody.

Dosing Regimens and Administration

The therapeutic methods and uses of the invention described herein include, in one aspect, administering to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) one or more dosing cycles of an effective amount of an anti-TIGIT antagonist antibody (e.g., an anti-TIGIT antagonist antibody as disclosed herein, e.g., tiragolumab) and an effective amount of an anti-PD-L1 antagonist antibody (e.g., atezolizumab), thereby treating the subject.

In some instances, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some instances, the effective amount of the anti-TIGIT antagonist antibody (e.g., an anti-TIGIT antagonist antibody as disclosed herein, e.g., tiragolumab) is a fixed dose of about 600 mg every three weeks. In some instances, effective amount of the anti-TIGIT antagonist antibody (e.g., an anti-TIGIT antagonist antibody as disclosed herein, e.g., tiragolumab) is a fixed dose of 600 mg every three weeks. In some instances, the fixed dose of the anti-TIGIT antagonist antibody (e.g., an anti-TIGIT antagonist antibody as disclosed herein, e.g., tiragolumab) administered in a combination therapy (e.g., a combination treatment with an anti-PD-L1 antagonist antibody, e.g., atezolizumab) may be reduced as compared to a standard dose of the anti-TIGIT antagonist antibody administered as a monotherapy.

In some instances, the effective amount of the anti-PD-L1 antagonist antibody (e.g., atezolizumab) is a fixed dose of between about 80 mg to about 1600 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200) every three weeks. In some instances, the effective amount of the anti-PD-L1 antagonist antibody (e.g., atezolizumab) is a fixed dose of about 1200 mg every three weeks. In some instances, the effective amount of the anti-PD-L1 antagonist antibody (e.g., atezolizumab) is a fixed dose of 1200 mg every three weeks. In some instances, the fixed dose of the anti-PD-L1 antagonist antibody (e.g., atezolizumab) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody administered as a monotherapy.

In some instances, the effective amount of the anti-PD-L1 antagonist antibody (e.g., atezolizumab) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some instances, the effective amount of the anti-PD-L1 antagonist antibody (e.g., atezolizumab) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, the effective amount of anti-PD-L1 antagonist antibody (e.g., atezolizumab) is a dose of about 15 mg/kg administered every three weeks. In some instances, the dose of the anti-PD-L1 antagonist antibody (e.g., atezolizumab) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody administered as a monotherapy.

In any of the methods and uses of the invention, the anti-TIGIT antagonist antibody (e.g., an anti-TIGIT antagonist antibody as disclosed herein, e.g., tiragolumab) and the anti-PD-L1 antagonist antibody (e.g., atezolizumab) may be administered in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In some instances, the dosing cycles of the anti-TIGIT antagonist antibody (e.g., an anti-TIGIT antagonist antibody as disclosed herein, e.g., tiragolumab) and the anti-PD-L1 antagonist antibody (e.g., atezolizumab) continue until there is a loss of clinical benefit (e.g., confirmed disease progression, drug resistance, death, or unacceptable toxicity). In some instances, the length of each dosing cycle is about 18 to 24 days (e.g., 15 days, 16 days, 17 days, 18 days, 19 days, 20 days, 21 days, 22 days, 23 days, or 24 days). In some instances, the length of each dosing cycle is about 21 days. In some instances, the anti-TIGIT antagonist antibody (e.g., an anti-TIGIT antagonist antibody as disclosed herein, e.g., tiragolumab) is administered on about Day 1 (e.g., Day 1±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 600 mg on Day 1 of each 21-day cycle (i.e., at a fixed dose of about 600 mg every three weeks). Similarly, in some instances, the anti-PD-L1 antagonist antibody (e.g., atezolizumab) is administered on about Day 1 (e.g., Day 1±3 days) of each dosing cycle. For example, the anti-PD-L1 antagonist antibody (e.g., atezolizumab) is administered intravenously at a fixed dose of about 1200 mg on Day 1 of each 21-day cycle (i.e., at a fixed dose of about 1200 mg every three weeks). In some instances, both the anti-TIGIT antagonist antibody (e.g., an anti-TIGIT antagonist antibody as disclosed herein, e.g., tiragolumab) and the anti-PD-L1 antagonist antibody (e.g., atezolizumab) are administered on about Day 1 (e.g., Day 1±3 days) of each dosing cycle. For example, the anti-TIGIT antagonist antibody (e.g., an anti-TIGIT antagonist antibody as disclosed herein, e.g., tiragolumab) is administered intravenously at a fixed dose of about 600 mg on Day 1 of each 21-day cycle (i.e., at a fixed dose of about 600 mg every three weeks), and the anti-PD-L1 antagonist antibody (e.g., atezolizumab) is administered intravenously at a fixed dose of about 1200 mg on Day 1 of each 21-day cycle (i.e., at a fixed dose of about 1200 mg every three weeks).

In some instances, the anti-TIGIT antagonist antibody (e.g., an anti-TIGIT antagonist antibody as disclosed herein, e.g., tiragolumab) is administered to the subject by intravenous infusion over about 60±10 minutes (e.g., about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, or about 70 minutes). In some instances, the anti-PD-L1 antagonist antibody (e.g., atezolizumab) is administered to the subject by intravenous infusion over about 60±15 minutes (e.g. about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes).

In some instances, the anti-TIGIT antagonist antibody (e.g., an anti-TIGIT antagonist antibody as disclosed herein, e.g., tiragolumab) is administered to the subject before the anti-PD-L1 antagonist antibody (e.g., atezolizumab). In some instances, for example, following administration of the anti-TIGIT antagonist antibody and before administration of the anti-PD-L1 antagonist antibody, the method includes an intervening first observation period. In some instances, the method further includes a second observation period following administration of the anti-PD-L1 antagonist antibody. In some instances, the method includes both a first observation period following administration of the anti-TIGIT antagonist antibody and second observation period following administration of the anti-PD-L1 antagonist antibody. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody and anti-PD-L1 antagonist antibody during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody and anti-PD-L1 antagonist antibody during the first and second observation periods, respectively.

In other instances, the anti-PD-L1 antagonist antibody (e.g. atezolizumab) is administered to the subject before the anti-TIGIT antagonist antibody (e.g., an anti-TIGIT antagonist antibody as disclosed herein, e.g., tiragolumab). In some instances, for example, following administration of the anti-PD-L1 antagonist antibody and before administration of the anti-TIGIT antagonist antibody, the method includes an intervening first observation period. In some instances, the method includes a second observation period following administration of the anti-TIGIT antagonist antibody. In some instances, the method includes both a first observation period following administration of the anti-PD-L1 antagonist antibody and second observation period following administration of the anti-TIGIT antagonist antibody. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the anti-PD-L1 antagonist antibody and anti-TIGIT antagonist antibody during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the anti-PD-L1 antagonist antibody and anti-TIGIT antagonist antibody during the first and second observation periods, respectively.

In other instances, the anti-TIGIT antagonist antibody (e.g., an anti-TIGIT antagonist antibody as disclosed herein, e.g., tiragolumab) and the anti-PD-L1 (atezolizumab) antagonist antibody are administered to the subject simultaneously. In some instances, for example, following administration of the anti-TIGIT antagonist antibody and the anti-PD-L1 antagonist antibody the method includes an observation period. In some instances the observation period is between about 30 minutes to about 60 minutes in length. In instances in which the observation period is about 60 minutes in length, the method may include recording the subject's vital signs (e.g., pulse rate, respiratory rate, blood pressure, and temperature) at about 30±10 minutes after administration of the anti-PD-L1 antagonist antibody and anti-TIGIT antagonist antibody during the observation period. In instances in which the observation period is about 30 minutes in length, the method may include recording the subject's vital signs (e.g., pulse rate, respiratory rate, blood pressure, and temperature) at about 15±10 minutes after administration of the anti-PD-L1 antagonist antibody and anti-TIGIT antagonist antibody during the observation period.

In another aspect, the invention provides a method of treating a subject having an NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) by administering to the subject one or more dosing cycles of an anti-TIGIT antagonist antibody at a fixed dose of 600 mg every three weeks and atezolizumab at a fixed dose of 1200 mg every three weeks, wherein the anti-TIGIT antagonist antibody has a VH domain having the amino acid sequence of SEQ ID NO: 17 or 18 and a VL domain having the amino acid sequence of SEQ ID NO: 19, as described in further detail below.

In another aspect, the invention provides a method of treating a subject having an NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) by administering to the subject one or more dosing cycles of tiragolumab at a fixed dose of 600 mg every three weeks and atezolizumab at a fixed dose of 1200 mg every three weeks.

In another aspect, the invention provides an anti-TIGIT antagonist antibody (e.g., an anti-TIGIT antagonist antibody disclosed herein, e.g., tiragolumab) and anti-PD-L1 antagonist antibody (e.g., atezolizumab) for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of an effective amount of an anti-TIGIT antagonist antibody and an effective amount of an anti-PD-L1 antagonist antibody.

In some instances, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, the effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some instances, the effective amount of the anti-TIGIT antagonist antibody (e.g., an anti-TIGIT antagonist antibody as disclosed herein, e.g., tiragolumab) is a fixed dose of about 600 mg every three weeks. In some instances, effective amount of the anti-TIGIT antagonist antibody (e.g., an anti-TIGIT antagonist antibody as disclosed herein, e.g., tiragolumab) is a fixed dose of 600 mg every three weeks. In some instances, the fixed dose of the anti-TIGIT antagonist antibody (e.g., an anti-TIGIT antagonist antibody as disclosed herein, e.g., tiragolumab) is to be administered in a combination therapy (e.g., a combination treatment with an anti-PD-L1 antagonist antibody, e.g., atezolizumab) may be reduced as compared to a standard dose of the anti-TIGIT antagonist antibody is to be administered as a monotherapy.

In some instances, the effective amount of the anti-PD-L1 antagonist antibody (e.g., atezolizumab) is a fixed dose of between about 80 mg to about 1600 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200) every three weeks. In some instances, the effective amount of the anti-PD-L1 antagonist antibody (e.g., atezolizumab) is a fixed dose of about 1200 mg every three weeks. In some instances, the effective amount of the anti-PD-L1 antagonist antibody (e.g., atezolizumab) is a fixed dose of 1200 mg every three weeks. In some instances, the fixed dose of the anti-PD-L1 antagonist antibody (e.g., atezolizumab) to be administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody to be administered as a monotherapy.

In some instances, the effective amount of the anti-PD-L1 antagonist antibody (e.g., atezolizumab) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some instances, the effective amount of the anti-PD-L1 antagonist antibody (e.g., atezolizumab) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, effective amount of anti-PD-L1 antagonist antibody (e.g., atezolizumab) is a dose of about 15 mg/kg to be administered every three weeks. In some instances, the dose of the anti-PD-L1 antagonist antibody (e.g., atezolizumab) is administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody administered as a monotherapy.

The anti-TIGIT antagonist antibody (e.g., an anti-TIGIT antagonist antibody as disclosed herein, e.g., tiragolumab) and the anti-PD-L1 antagonist antibody (e.g., atezolizumab) may be administered in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In some instances, the dosing cycles of the anti-TIGIT antagonist antibody (e.g., an anti-TIGIT antagonist antibody as disclosed herein, e.g., tiragolumab) and the anti-PD-L1 antagonist antibody (e.g., atezolizumab) continue until there is a loss of clinical benefit (e.g., confirmed disease progression, drug resistance, death, or unacceptable toxicity). In some instances, the length of each dosing cycle is about 18 to 24 days (e.g., 15 days, 16 days, 17 days, 18 days, 19 days, 20 days, 21 days, 22 days, 23 days, or 24 days). In some instances, the length of each dosing cycle is about 21 days. In some instances, the anti-TIGIT antagonist antibody (e.g., an anti-TIGIT antagonist antibody as disclosed herein, e.g., tiragolumab) is to be administered on about Day 1 (e.g., Day 1±3 days) of each dosing cycle. For example, the anti-TIGIT antagonist antibody (e.g., an anti-TIGIT antagonist antibody as disclosed herein, e.g., tiragolumab) is to be administered intravenously at a fixed dose of about 600 mg on Day 1 of each 21-day cycle (i.e., at a fixed dose of about 600 mg every three weeks). Similarly, in some instances, the anti-PD-L1 antagonist antibody (e.g., atezolizumab) is to be administered on about Day 1 (e.g., Day 1±3 days) of each dosing cycle. For example, the anti-PD-L1 antagonist antibody (e.g., atezolizumab) is to be administered intravenously at a fixed dose of about 1200 mg on Day 1 of each 21-day cycle (i.e., at a fixed dose of about 1200 mg every three weeks). In some instances, both the anti-TIGIT antagonist antibody (e.g., an anti-TIGIT antagonist antibody as disclosed herein, e.g., tiragolumab) and the anti-PD-L1 antagonist antibody (e.g., atezolizumab) are to be administered on about Day 1 (e.g., Day 1±3 days) of each dosing cycle. For example, the anti-TIGIT antagonist antibody (e.g., an anti-TIGIT antagonist antibody as disclosed herein, e.g., tiragolumab) is to be administered intravenously at a fixed dose of about 600 mg on Day 1 of each 21-day cycle (i.e., at a fixed dose of about 600 mg every three weeks), and the anti-PD-L1 antagonist antibody (e.g., atezolizumab) is to be administered intravenously at a fixed dose of about 1200 mg on Day 1 of each 21-day cycle (i.e., at a fixed dose of about 1200 mg every three weeks).

In some instances, the anti-TIGIT antagonist antibody (e.g., an anti-TIGIT antagonist antibody as disclosed herein, e.g., tiragolumab) is to be administered to the subject by intravenous infusion over about 60±10 minutes (e.g., about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, or about 70 minutes). In some instances, the anti-PD-L1 antagonist antibody (e.g., atezolizumab) is to be administered to the subject by intravenous infusion over about 60±15 minutes (e.g. about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes).

In some instances, the anti-TIGIT antagonist antibody (e.g., an anti-TIGIT antagonist antibody as disclosed herein, e.g., tiragolumab) is to be administered to the subject before the anti-PD-L1 antagonist antibody (e.g., atezolizumab). In some instances, for example, following administration of the anti-TIGIT antagonist antibody and before administration of the anti-PD-L1 antagonist antibody, the method includes an intervening first observation period. In some instances, the method further includes a second observation period following administration of the anti-PD-L1 antagonist antibody. In some instances, the method includes both a first observation period following administration of the anti-TIGIT antagonist antibody and second observation period following administration of the anti-PD-L1 antagonist antibody. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the anti-TIGIT antagonist antibody and anti-PD-L1 antagonist antibody during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the anti-TIGIT antagonist antibody and anti-PD-L1 antagonist antibody during the first and second observation periods, respectively.

In other instances, the anti-PD-L1 antagonist antibody (e.g. atezolizumab) is to be administered to the subject before the anti-TIGIT antagonist antibody (e.g., an anti-TIGIT antagonist antibody as disclosed herein, e.g., tiragolumab). In some instances, for example, following administration of the anti-PD-L1 antagonist antibody and before administration of the anti-TIGIT antagonist antibody, the method includes an intervening first observation period. In some instances, the method includes a second observation period following administration of the anti-TIGIT antagonist antibody. In some instances, the method includes both a first observation period following administration of the anti-PD-L1 antagonist antibody and second observation period following administration of the anti-TIGIT antagonist antibody. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the anti-PD-L1 antagonist antibody and anti-TIGIT antagonist antibody during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the anti-PD-L1 antagonist antibody and anti-TIGIT antagonist antibody during the first and second observation periods, respectively.

In other instances, the anti-TIGIT antagonist antibody (e.g., an anti-TIGIT antagonist antibody as disclosed herein, e.g., tiragolumab) and the anti-PD-L1 (atezolizumab) antagonist antibody is to be administered to the subject simultaneously. In some instances, for example, following administration of the anti-TIGIT antagonist antibody and the anti-PD-L1 antagonist antibody the method includes an observation period. In some instances the observation period is between about 30 minutes to about 60 minutes in length.

In instances in which the observation period is about 60 minutes in length, the method may include recording the subject's vital signs (e.g., pulse rate, respiratory rate, blood pressure, and temperature) at about 30±10 minutes after administration of the anti-PD-L1 antagonist antibody and anti-TIGIT antagonist antibody during the observation period. In instances in which the observation period is about 30 minutes in length, the method may include recording the subject's vital signs (e.g., pulse rate, respiratory rate, blood pressure, and temperature) at about 15±10 minutes after administration of the anti-PD-L1 antagonist antibody and anti-TIGIT antagonist antibody during the observation period.

In another aspect, the invention provides an anti-TIGIT antagonist antibody (e.g., an anti-TIGIT antagonist antibody disclosed herein, e.g., tiragolumab) and anti-PD-L1 antagonist antibody (e.g., atezolizumab) for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of an anti-TIGIT antagonist antibody at a fixed dose of 600 mg every three weeks and atezolizumab at a fixed dose of 1200 mg every three weeks, wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides an anti-TIGIT antagonist antibody (e.g., an anti-TIGIT antagonist antibody disclosed herein, e.g., tiragolumab) and anti-PD-L1 antagonist antibody (e.g., atezolizumab) for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of tiragolumab at a fixed dose of 600 mg every three weeks and atezolizumab at a fixed dose of 1200 mg every three weeks.

In another aspect, the invention provides uses of an anti-TIGIT antagonist antibody (e.g., an anti-TIGIT antagonist antibody disclosed herein, e.g., tiragolumab) and anti-PD-L1 antagonist antibody (e.g., atezolizumab) in the manufacture or preparation of a medicament for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of the medicament, and wherein the medicament is formulated for administration of an effective amount of the anti-TIGIT antagonist antibody and an effective amount of the anti-PD-L1 antagonist.

In another aspect, the invention provides uses of an anti-TIGIT antagonist antibody in the manufacture of a medicament for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of the medicament and an anti-PD-L1 antagonist antibody, and wherein the medicament is formulated for administration of an effective amount of the anti-TIGIT antagonist antibody and an effective amount of the anti-PD-L1 antagonist antibody.

In another aspect, the invention provides uses of an anti-PD-L1 antagonist antibody in the manufacture of a medicament for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of the medicament and an anti-TIGIT antagonist antibody, and wherein the medicament is formulated for administration an effective amount of the anti-PD-L1 antagonist antibody and an effective amount of the anti-TIGIT antagonist antibody is to be administered.

In some instances, the effective amount of the anti-TIGIT antagonist antibody (e.g., an anti-TIGIT antagonist antibody as disclosed herein, e.g., tiragolumab) is a fixed dose of between about 30 mg to about 1200 mg (e.g., between about 30 mg to about 1100 mg, e.g., between about 60 mg to about 1000 mg, e.g., between about 100 mg to about 900 mg, e.g., between about 200 mg to about 800 mg, e.g., between about 300 mg to about 800 mg, e.g., between about 400 mg to about 800 mg, e.g., between about 400 mg to about 750 mg, e.g., between about 450 mg to about 750 mg, e.g., between about 500 mg to about 700 mg, e.g., between about 550 mg to about 650 mg, e.g., 600 mg±10 mg, e.g., 600±6 mg, e.g., 600±5 mg, e.g., 600±3 mg, e.g., 600±1 mg, e.g., 600±0.5 mg, e.g., 600 mg) every three weeks. In some instances, an effective amount of the anti-TIGIT antagonist antibody (e.g., an anti-TIGIT antagonist antibody as disclosed herein, e.g., tiragolumab) is a fixed dose of between about 30 mg to about 600 mg (e.g., between about 50 mg to between 600 mg, e.g., between about 60 mg to about 600 mg, e.g., between about 100 mg to about 600 mg, e.g., between about 200 mg to about 600 mg, e.g., between about 200 mg to about 550 mg, e.g., between about 250 mg to about 500 mg, e.g., between about 300 mg to about 450 mg, e.g., between about 350 mg to about 400 mg, e.g., about 375 mg) every three weeks. In some instances, the effective amount of the anti-TIGIT antagonist antibody (e.g., an anti-TIGIT antagonist antibody as disclosed herein, e.g., tiragolumab) is a fixed dose of about 600 mg every three weeks. In some instances, effective amount of the anti-TIGIT antagonist antibody (e.g., an anti-TIGIT antagonist antibody as disclosed herein, e.g., tiragolumab) is a fixed dose of 600 mg every three weeks. In some instances, the fixed dose of the anti-TIGIT antagonist antibody (e.g., an anti-TIGIT antagonist antibody as disclosed herein, e.g., tiragolumab) is to be administered in a combination therapy (e.g., a combination treatment with an anti-PD-L1 antagonist antibody, e.g., atezolizumab) may be reduced as compared to a standard dose of the anti-TIGIT antagonist antibody is to be administered as a monotherapy.

In some instances, the effective amount of the anti-PD-L1 antagonist antibody (e.g., atezolizumab) is a fixed dose of between about 80 mg to about 1600 mg (e.g., between about 100 mg to about 1600 mg, e.g., between about 200 mg to about 1600 mg, e.g., between about 300 mg to about 1600 mg, e.g., between about 400 mg to about 1600 mg, e.g., between about 500 mg to about 1600 mg, e.g., between about 600 mg to about 1600 mg, e.g., between about 700 mg to about 1600 mg, e.g., between about 800 mg to about 1600 mg, e.g., between about 900 mg to about 1500 mg, e.g., between about 1000 mg to about 1400 mg, e.g., between about 1050 mg to about 1350 mg, e.g., between about 1100 mg to about 1300 mg, e.g., between about 1150 mg to about 1250 mg, e.g., between about 1175 mg to about 1225 mg, e.g., between about 1190 mg to about 1210 mg, e.g., 1200 mg±5 mg, e.g., 1200±2.5 mg, e.g., 1200±1.0 mg, e.g., 1200±0.5 mg, e.g., 1200) every three weeks. In some instances, the effective amount of the anti-PD-L1 antagonist antibody (e.g., atezolizumab) is a fixed dose of about 1200 mg every three weeks. In some instances, the effective amount of the anti-PD-L1 antagonist antibody (e.g., atezolizumab) is a fixed dose of 1200 mg every three weeks. In some instances, the fixed dose of the anti-PD-L1 antagonist antibody (e.g., atezolizumab) to be administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody to be administered as a monotherapy.

In some instances, the effective amount of the anti-PD-L1 antagonist antibody (e.g., atezolizumab) is a dose of between about 0.01 mg/kg to about 50 mg/kg of the subject's body weight (e.g., between about 0.01 mg/kg to about 45 mg/kg, e.g., between about 0.1 mg/kg to about 40 mg/kg, e.g., between about 1 mg/kg to about 35 mg/kg, e.g., between about 2.5 mg/kg to about 30 mg/kg, e.g., between about 5 mg/kg to about 25 mg/kg, e.g., between about 10 mg/kg to about 20 mg/kg, e.g., between about 12.5 mg/kg to about 15 mg/kg, e.g., about 15±2 mg/kg, about 15±1 mg/kg, about 15±0.5 mg/kg, about 15±0.2 mg/kg, or about 15±0.1 mg/kg, e.g., about 15 mg/kg) every three weeks. In some instances, the effective amount of the anti-PD-L1 antagonist antibody (e.g., atezolizumab) is a dose of between about 0.01 mg/kg to about 15 mg/kg of the subject's body weight (e.g., between about 0.1 mg/kg to about 15 mg/kg, e.g., between about 0.5 mg/kg to about 15 mg/kg, e.g., between about 1 mg/kg to about 15 mg/kg, e.g., between about 2.5 mg/kg to about 15 mg/kg, e.g., between about 5 mg/kg to about 15 mg/kg, e.g., between about 7.5 mg/kg to about 15 mg/kg, e.g., between about 10 mg/kg to about 15 mg/kg, e.g., between about 12.5 mg/kg to about 15 mg/kg, e.g., between about 14 mg/kg to about 15 mg/kg, e.g., about 15±1 mg/kg, e.g., about 15±0.5 mg/kg, e.g., about 15±0.2 mg/kg, e.g., about 15±0.1 mg/kg, e.g., about 15 mg/kg) every three weeks. In some instances, the effective amount of anti-PD-L1 antagonist antibody (e.g., atezolizumab) is a dose of about 15 mg/kg to be administered every three weeks. In some instances, the dose of the anti-PD-L1 antagonist antibody (e.g., atezolizumab) administered in a combination therapy (e.g., a combination treatment with an anti-TIGIT antagonist antibody, such as an anti-TIGIT antagonist antibody disclosed herein, e.g., tiragolumab) may be reduced as compared to a standard dose of the anti-PD-L1 antagonist antibody administered as a monotherapy.

In any of the uses of the invention, the medicament comprising the anti-TIGIT antagonist antibody (e.g., an anti-TIGIT antagonist antibody as disclosed herein, e.g., tiragolumab) and the anti-PD-L1 antagonist antibody (e.g., atezolizumab) may be administered in one or more dosing cycles (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more dosing cycles). In some instances, the dosing cycles of the medicament comprising anti-TIGIT antagonist antibody (e.g., an anti-TIGIT antagonist antibody as disclosed herein, e.g., tiragolumab) and the anti-PD-L1 antagonist antibody (e.g., atezolizumab) continue until there is a loss of clinical benefit (e.g., confirmed disease progression, drug resistance, death, or unacceptable toxicity). In some instances, the length of each dosing cycle is about 18 to 24 days (e.g., 15 days, 16 days, 17 days, 18 days, 19 days, 20 days, 21 days, 22 days, 23 days, or 24 days). In some instances, the length of each dosing cycle is about 21 days. In some instances, the medicament comprising the anti-TIGIT antagonist antibody (e.g., an anti-TIGIT antagonist antibody as disclosed herein, e.g., tiragolumab) is to be administered on about Day 1 (e.g., Day 1±3 days) of each dosing cycle. For example, the medicament comprising the anti-TIGIT antagonist antibody (e.g., an anti-TIGIT antagonist antibody as disclosed herein, e.g., tiragolumab) is to be administered intravenously at a fixed dose of about 600 mg on Day 1 of each 21-day cycle (i.e., at a fixed dose of about 600 mg every three weeks). Similarly, in some instances, the medicament comprising the anti-PD-L1 antagonist antibody (e.g., atezolizumab) is to be administered on about Day 1 (e.g., Day 1±3 days) of each dosing cycle. For example, the medicament comprising the anti-PD-L1 antagonist antibody (e.g., atezolizumab) is to be administered intravenously at a fixed dose of about 1200 mg on Day 1 of each 21-day cycle (i.e., at a fixed dose of about 1200 mg every three weeks). In some instances, the medicament comprising both the anti-TIGIT antagonist antibody (e.g., an anti-TIGIT antagonist antibody as disclosed herein, e.g., tiragolumab) and the anti-PD-L1 antagonist antibody (e.g., atezolizumab) are to be administered on about Day 1 (e.g., Day 1±3 days) of each dosing cycle. For example, the medicament comprising the anti-TIGIT antagonist antibody (e.g., an anti-TIGIT antagonist antibody as disclosed herein, e.g., tiragolumab) is to be administered intravenously at a fixed dose of about 600 mg on Day 1 of each 21-day cycle (i.e., at a fixed dose of about 600 mg every three weeks), and the medicament comprising the anti-PD-L1 antagonist antibody (e.g., atezolizumab) is to be administered intravenously at a fixed dose of about 1200 mg on Day 1 of each 21-day cycle (i.e., at a fixed dose of about 1200 mg every three weeks).

In some instances, the medicament comprising the anti-TIGIT antagonist antibody (e.g., an anti-TIGIT antagonist antibody as disclosed herein, e.g., tiragolumab) is administered to the subject by intravenous infusion over about 60±10 minutes (e.g., about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, or about 70 minutes). In some instances, the medicament comprising the anti-PD-L1 antagonist antibody (e.g., atezolizumab) is to be administered to the subject by intravenous infusion over about 60±15 minutes (e.g. about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, or about 75 minutes).

In some instances, the medicament comprising the anti-TIGIT antagonist antibody (e.g., an anti-TIGIT antagonist antibody as disclosed herein, e.g., tiragolumab) is to be administered to the subject before the medicament comprising the anti-PD-L1 antagonist antibody (e.g., atezolizumab). In some instances, for example, following administration of the medicament comprising the anti-TIGIT antagonist antibody and before administration of the medicament comprising the anti-PD-L1 antagonist antibody, the method includes an intervening first observation period. In some instances, the method further includes a second observation period following administration of the anti-PD-L1 antagonist antibody. In some instances, the method includes both a first observation period following administration of the medicament comprising the anti-TIGIT antagonist antibody and second observation period following administration of the medicament comprising the anti-PD-L1 antagonist antibody. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the medicament comprising the anti-TIGIT antagonist antibody and the medicament comprising the anti-PD-L1 antagonist antibody during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the medicament comprising the anti-TIGIT antagonist antibody and the medicament comprising the anti-PD-L1 antagonist antibody during the first and second observation periods, respectively.

In other instances, the medicament comprising the anti-PD-L1 antagonist antibody (e.g. atezolizumab) is to be administered to the subject before the anti-TIGIT antagonist antibody (e.g., an anti-TIGIT antagonist antibody as disclosed herein, e.g., tiragolumab). In some instances, for example, following administration of the medicament comprising the anti-PD-L1 antagonist antibody and before administration of the medicament comprising the anti-TIGIT antagonist antibody, the method includes an intervening first observation period. In some instances, the method includes a second observation period following administration of the medicament comprising the anti-TIGIT antagonist antibody. In some instances, the method includes both a first observation period following administration of the medicament comprising the anti-PD-L1 antagonist antibody and second observation period following administration of the medicament comprising the anti-TIGIT antagonist antibody. In some instances, the first and second observation periods are each between about 30 minutes to about 60 minutes in length. In instances in which the first and second observation periods are each about 60 minutes in length, the method may include recording the subject's vital signs (e.g., pulse rate, respiratory rate, blood pressure, and temperature) at about 30±10 minutes after administration of the medicament comprising the anti-PD-L1 antagonist antibody and the medicament comprising the anti-TIGIT antagonist antibody during the first and second observation periods, respectively. In instances in which the first and second observation periods are each about 30 minutes in length, the method may include recording the subject's vital signs (e.g., pulse rate, respiratory rate, blood pressure, and temperature) at about 15±10 minutes after administration of the medicament comprising the anti-PD-L1 antagonist antibody and the medicament comprising the anti-TIGIT antagonist antibody during the first and second observation periods, respectively.

In other instances, the medicament comprising the anti-TIGIT antagonist antibody (e.g., an anti-TIGIT antagonist antibody as disclosed herein, e.g., tiragolumab) and the medicament comprising the anti-PD-L1 (atezolizumab) antagonist antibody is to be administered to the subject simultaneously. In some instances, for example, following administration of the medicament comprising the anti-TIGIT antagonist antibody and the medicament comprising the anti-PD-L1 antagonist antibody the method includes an observation period. In some instances the observation period is between about 30 minutes to about 60 minutes in length. In instances in which the observation period is about 60 minutes in length, the method may include recording the subject's vital signs (e.g., pulse rate, respiratory rate, blood pressure, and temperature) at about 30±10 minutes after administration of the medicament comprising the anti-PD-L1 antagonist antibody and the medicament comprising the anti-TIGIT antagonist antibody during the observation period. In instances in which the observation period is about 30 minutes in length, the method may include recording the subject's vital signs (e.g., pulse rate, respiratory rate, blood pressure, and temperature) at about 15±10 minutes after administration of the medicament comprising the anti-PD-L1 antagonist antibody and the medicament comprising the anti-TIGIT antagonist antibody during the observation period.

In another aspect, the invention provides uses of an anti-TIGIT antagonist antibody (e.g., an anti-TIGIT antagonist antibody disclosed herein, e.g., tiragolumab) and anti-PD-L1 antagonist antibody (e.g., atezolizumab) in the manufacture or preparation of a medicament for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of the medicament, and wherein the medicament is formulated for administration of the anti-TIGIT antagonist antibody at a fixed dose of between about 30 mg to about 1200 mg every three weeks and the anti-PD-L1 antagonist antibody at a fixed dose of between about 80 mg to about 1600 mg every three weeks.

In another aspect, the invention provides uses of an anti-PD-L1 antagonist antibody in the manufacture of a medicament for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of the medicament and an anti-TIGIT antagonist antibody, and wherein the medicament is formulated for administration of the anti-PD-L1 antagonist antibody at a fixed dose of between about 80 mg to about 1600 mg every three weeks and the anti-TIGIT antagonist antibody is to be administered at a fixed dose of between about 30 mg to about 1200 mg every three weeks.

In another aspect, the invention provides uses of an anti-TIGIT antagonist antibody in the manufacture of a medicament for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of the medicament and an anti-PD-L1 antagonist antibody, and wherein the medicament is formulated for administration of the anti-TIGIT antagonist antibody at a fixed dose of between about 30 mg to about 1200 mg every three weeks and the anti-PD-L1 antagonist antibody at a fixed dose of between about 80 mg to about 1600 mg every three weeks.

In another aspect, the invention provides uses of an anti-TIGIT antagonist antibody and atezolizumab in the manufacture of a medicament for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of the medicament, wherein the medicament is formulated for administration of the anti-TIGIT antagonist antibody at a fixed dose of 600 mg every three weeks and atezolizumab at a fixed dose of 1200 mg every three weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of an anti-TIGIT antagonist antibody in the manufacture of a medicament for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of the medicament and atezolizumab, wherein the medicament is formulated for administration of the anti-TIGIT antagonist antibody at a fixed dose of 600 mg every three weeks and atezolizumab is to be administered at a fixed dose of 1200 mg every three weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of atezolizumab in the manufacture of a medicament for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of the medicament and an anti-TIGIT antibody, wherein the medicament is formulated for administration of atezolizumab at a fixed dose of 1200 mg every three weeks and the anti-TIGIT antagonist antibody is to be administered at a fixed dose of 600 mg every three weeks, and wherein the anti-TIGIT antagonist antibody comprises: a VH domain comprising the amino acid sequence of SEQ ID NO: 17 or 18; and a VL domain comprising the amino acid sequence of SEQ ID NO: 19, as described in further detail below.

In another aspect, the invention provides uses of tiragolumab and atezolizumab in the manufacture of a medicament for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of the medicament, wherein the medicament is formulated for administration of tiragolumab at a fixed dose of 600 mg every three weeks and atezolizumab at a fixed dose of 1200 mg every three weeks.

In another aspect, the invention provides uses of tiragolumab in the manufacture of a medicament for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of the medicament and atezolizumab, wherein the medicament is formulated for administration of tiragolumab at a fixed dose of 600 mg every three weeks and atezolizumab is to be administered at a fixed dose of 1200 mg every three weeks.

In another aspect, the invention provides uses of atezolizumab in the manufacture of a medicament for use in a method of treating a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein the method comprises administering to the subject one or more dosing cycles of the medicament and tiragolumab, wherein the medicament is formulated for administration of atezolizumab at a fixed dose of 1200 mg every three weeks and tiragolumab is to be administered at a fixed dose of 600 mg every three weeks.

In any of the methods, uses, or compositions for use described herein, the anti-TIGIT antagonist antibody (e.g., an anti-TIGIT antagonist antibody as disclosed herein, e.g., tiragolumab) and anti-PD-L1 antibody (e.g., atezolizumab), or a medicament thereof, may be administered in conjunction with (either separately or together), one or more additional anti-cancer therapeutic agent(s) (e.g., a chemotherapeutic agent, a cytotoxic agent, a growth inhibitory agent, a radiotherapy/radiation therapy, and/or an anti-hormonal agent, such as those recited herein above).

In any of the methods, uses, or compositions for use described herein, the anti-TIGIT antagonist antibody (e.g., an anti-TIGIT antagonist antibody as disclosed herein, e.g., tiragolumab) and anti-PD-L1 antibody (e.g., atezolizumab), or a medicament thereof, is for treating a subject having a lung cancer. In some instances, the lung cancer is a NSCLC. The cancer may be at an early or late stage. In some instances, the NSCLC is a squamous NSCLC. In some instances, the NSCLC is a non-squamous NSCLC. In some instances, the NSCLC is a locally advanced unresectable NSCLC. In some instances, the NSCLC is a Stage IIIB NSCLC. In some instances, the NSCLC is a recurrent or metastatic NSCLC. In some instances, the NSCLC is a Stage IV NSCLC. In some instances, the subject has not been previously treated for Stage IV NSCLC.

In some instances, in any of the methods, uses, or compositions for use described herein, the subject does not have a sensitizing epidermal growth factor receptor (EGFR) gene mutation or anaplastic lymphoma kinase (ALK) gene rearrangement. In some instances, the subject has an Eastern Cooperative Oncology Group (ECOG) Performance Status (PS) of 0 or 1.

In some instances, in any of the methods, uses, or compositions for use described herein, the subject does not have a pulmonary lymphoepithelioma-like carcinoma subtype of NSCLC.

In some instances, in any of the methods, uses, or compositions for use described herein, the subject does not have an active Epstein-Barr virus (EBV) infection or a known or suspected chronic active EBV infection. In some instances, the subject is negative for EBV IgM and/or negative by EBV PCR. In some instances, the subject is negative for EBV IgM and/or negative by EBV PCR and is positive for EBV IgG and/or positive for Epstein-Barr nuclear antigen (EBNA). In other instances, the subject is negative for EBV IgG and/or negative for EBNA.

In some instances, in any of the methods, uses, or compositions for use described herein, the subject has a PD-L1 selected tumor (e.g., a tumor PD-L1 expression with a minimum TPS 1% as determined by an IHC with the 22C3 antibody). In some instances the PD-L1 selected tumor is a tumor that has been determined to have a detectable protein expression level of PD-L1 by an immunohistochemical (IHC) assay. In some instances, the IHC assay uses the anti-PD-L1 antibody 22C3, SP142, SP263, or 28-8. In some instances, the IHC assay uses anti-PD-L1 antibody 22C3. In some instances, the tumor sample has been determined to have a tumor proportion score (TPS) of greater than, or equal to, 1%. In some instances, the TPS is greater than, or equal to, 1% and less than 50%. In some instances, the TPS is greater than, or equal to, 50%.

In some instances, in any of the methods, uses, or compositions for use described herein, the IHC assay uses anti-PD-L1 antibody SP142. In some instances, the tumor sample has been determined to have a detectable expression level of PD-L1 in greater than, or equal to, 1% of the tumor cells in the tumor sample. In some instances, the tumor sample has been determined to have a detectable expression level of PD-L1 in greater than, or equal to, 1% and less than 5% of the tumor cells in the tumor sample. In some instances, the tumor sample has been determined to have a detectable expression level of PD-L1 in greater than, or equal to, 5% and less than 50% of the tumor cells in the tumor sample. In some instances, the tumor sample has been determined to have a detectable expression level of PD-L1 in greater than, or equal to, 50% of the tumor cells in the tumor sample. In some instances, the tumor sample has been determined to have a detectable expression level of PD-L1 in tumor-infiltrating immune cells that comprise greater than, or equal to, 1% of the tumor sample. In some instances, the tumor sample has been determined to have a detectable expression level of PD-L1 in tumor-infiltrating immune cells that comprise greater than, or equal to, 1% and less than 5% of the tumor sample. In some instances, the tumor sample has been determined to have a detectable expression level of PD-L1 in tumor-infiltrating immune cells that comprise greater than, or equal to, 5% and less than 10% of the tumor sample. In some instances, the tumor sample has been determined to have a detectable expression level of PD-L1 in tumor-infiltrating immune cells that comprise greater than, or equal to, 10% of the tumor sample.

In some instances, in any of the methods, uses, or compositions for use described herein, the detectable expression level of PD-L1 is a detectable nucleic acid expression level of PD-L1. In some instances, the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

In some instances, in any of the methods, uses, or compositions for use described herein, administration of the anti-TIGIT antagonist antibody and anti-PD-L1 antagonist antibody results in a clinical response. In some instances, the clinical response is an increase in the objective response rate (ORR) of the subject as compared to a reference ORR. In some instances, the reference ORR is the median ORR of a population of subjects who have received a treatment comprising an anti-PD-L1 antagonist antibody without an anti-TIGIT antagonist antibody. In some instances, the clinical response is an increase in the progression-free survival (PFS) of the subject as compared to a reference PFS time. In some instances, wherein the reference PFS time is the median PFS time of a population of subjects who have received a treatment comprising an anti-PD-L1 antagonist antibody without an anti-TIGIT antagonist antibody.

### IV. Diagnostic Methods and Uses

The invention provides methods for selecting a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein therapy is guided by diagnostic methods that involve determining the presence and/or expression levels/amount of one or more biomarkers in a sample obtained from the subject.

Additionally provided herein are methods for identifying a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who may benefit from a treatment comprising an anti-TIGIT antagonist antibody and an anti-PD-L1 antagonist antibody, wherein identification is guided by diagnostic methods that involve determining the presence and/or expression levels/amount of one or more biomarkers in a sample obtained from the subject.

Additionally provided herein are methods for assessing responsiveness to a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein further therapy is guided by diagnostic methods that involve determining the presence and/or expression levels/amount of one or more biomarkers in a sample obtained from the subject.

Additionally provided herein are methods for optimizing a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein further therapy is guided by diagnostic methods that involve determining the presence and/or expression levels/amount of one or more biomarkers in a sample obtained from the subject.

Biomarkers for use in the methods described herein can include, but are not limited to, PD-L1 and TIGIT expression on tumor tissues, germline and somatic mutations from tumor tissue and/or from circulating tumor DNA in blood (including, but not limited to, mutation load, MSI, and MMR defects), identified through WGS and/or NGS, and plasma derived cytokines. In some instances, the biomarker is PD-L1.

In some instances, the method includes determining the presence and/or expression levels/amount of a biomarker (e.g., PD-L1) in a sample from the subject, and administering to the subject one or more dosing cycles of an anti-TIGIT antagonist antibody (e.g., an anti-TIGIT antagonist antibody disclosed herein, e.g., tiragolumab) at a fixed dose of between about 30 mg to about 1200 mg every three weeks and one or more dosing cycles of an anti-PD-L1 antagonist antibody, e.g., atezolizumab) at a fixed dose of between about 80 mg to about 1600 mg every three weeks. In some instances, the method includes determining the presence and/or expression levels/amount of a biomarker (e.g., PD-L1) in a sample from the subject, and administering to the subject one or more dosing cycles of an anti-TIGIT antagonist antibody (e.g., an anti-TIGIT antagonist antibody disclosed herein, e.g., tiragolumab) at a fixed dose of about 600 mg every three weeks and one or more dosing cycles of an anti-PD-L1 antagonist antibody, e.g., atezolizumab) at a fixed dose of 1200 mg every three weeks.

Presence and/or expression levels/amount of a biomarker (e.g., PD-L1) can be determined qualitatively and/or quantitatively based on any suitable criterion known in the art, including but not limited to proteins, protein fragments, DNA, mRNA, cDNA, and/or gene copy number.

In some instances, expression levels or amount of a biomarker is a detectable protein expression level of PD-L1 in a tumor sample from the subject. In some instances, the PD-L1 protein expression level has been determined by an immunohistochemical (IHC) assay. In some instances the IHC assay uses the anti-PD-L1 antibody 22C3, SP142, SP263, or 28-8. In a particular instances, the IHC assay uses the anti-PD-L1 antibody 22C3. In some instances, the tumor sample has been determined to have a tumor proportion score (TPS) of greater than, or equal to, about 1% (e.g., about 1% or more, about 2% or more, about 3% or more, about 4% or more, about 5% or more, about 10% or more, about 15% or more, about 20% or more, about 25% or more, about 30% or more, about 35% or more, about 40% or more, about 50% or more, about 55% or more, about 60% or more, about 65% or more, about 70% or more, about 80% or more, about 85% or more, about 90% or more, about 95% or more, or about 99% or more). For example, in some instances, the tumor sample has a detectable protein expression level of PD-L1 with a TPS of about 1% to less than about 99% (e.g., about 1% to less than about 95%, about 1% to less than about 90%, about 1% to less than about 85%, about 1% to less than about 80%, about 1% to less than about 75%, about 1% to less than about 70%, about 1% to less than about 65%, about 1% to less than about 60%, about 1% to less than about 55%, about 1% to less than about 50%, about 1% to less than about 40%, about 1% to less than about 35%, about 1% to less than about 30%, about 1% to less than about 25%, about 1% to less than about 20%, about 1% to less than about 15%, about 1% to less than about 10%, about 1% to less than about 5%, about 5% to less than about 95%, about 5% to less than about 90%, about 5% to less than about 85%, about 5% to less than about 80%, about 5% to less than about 75%, about 5% to less than about 70%, about 5% to less than about 65%, about 5% to less than about 60%, about 5% to less than about 55%, about 5% to less than about 50%, about 5% to less than about 40%, about 5% to less than about 35%, about 5% to less than about 30%, about 5% to less than about 25%, about 5% to less than about 20%, about 5% to less than about 15%, about 5% to less than about 10%, about 10% to less than about 95%, about 10% to less than about 90%, about 10% to less than about 85%, about 10% to less than about 80%, about 10% to less than about 75%, about 10% to less than about 70%, about 10% to less than about 65%, about 10% to less than about 60%, about 10% to less than about 55%, about 10% to less than about 50%, about 10% to less than about 40%, about 10% to less than about 35%, about 10% to less than about 30%, about 10% to less than about 25%, about 10% to less than about 20%, about 10% to less than about 15%). In some instances, the TPS is greater than, or equal to 1%, and less than 50% (e.g., about 1% to about 49%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, or about 1% to about 2.5%). In some instances, the TPS is greater than, or equal to, 50% (e.g., about 50% to about 99%, about 50% to about 90%, about 50% to about 85%, about 50% to about 80%, about 50% to about 75%, about 50% to about 70%, about 50% to about 65%, about 50% to about 60%, or about 50% to about 55%).

In some instances, the IHC assay uses the anti-PD-L1 antibody SP142. In some instances, the tumor sample from the subject has been determined to have a detectable expression level of PD-L1 in greater than, or equal to, 1% (e.g., about 1% or more, about 2% or more, about 3% or more, about 4% or more, about 5% or more, about 10% or more, about 15% or more, about 20% or more, about 25% or more, about 30% or more, about 35% or more, about 40% or more, about 50% or more, about 55% or more, about 60% or more, about 65% or more, about 70% or more, about 80% or more, about 85% or more, about 90% or more, about 95% or more, or about 99% or more) of the tumor cells in the tumor sample, for example, by area. For example, in some instances, the tumor sample has a detectable expression level of PD-L1 in tumor cells that comprise about 1% to less than about 99% (e.g., about 1% to less than about 95%, about 1% to less than about 90%, about 1% to less than about 85%, about 1% to less than about 80%, about 1% to less than about 75%, about 1% to less than about 70%, about 1% to less than about 65%, about 1% to less than about 60%, about 1% to less than about 55%, about 1% to less than about 50%, about 1% to less than about 40%, about 1% to less than about 35%, about 1% to less than about 30%, about 1% to less than about 25%, about 1% to less than about 20%, about 1% to less than about 15%, about 1% to less than about 10%, about 1% to less than about 5%, about 5% to less than about 95%, about 5% to less than about 90%, about 5% to less than about 85%, about 5% to less than about 80%, about 5% to less than about 75%, about 5% to less than about 70%, about 5% to less than about 65%, about 5% to less than about 60%, about 5% to less than about 55%, about 5% to less than about 50%, about 5% to less than about 40%, about 5% to less than about 35%, about 5% to less than about 30%, about 5% to less than about 25%, about 5% to less than about 20%, about 5% to less than about 15%, about 5% to less than about 10%, about 10% to less than about 95%, about 10% to less than about 90%, about 10% to less than about 85%, about 10% to less than about 80%, about 10% to less than about 75%, about 10% to less than about 70%, about 10% to less than about 65%, about 10% to less than about 60%, about 10% to less than about 55%, about 10% to less than about 50%, about 10% to less than about 40%, about 10% to less than about 35%, about 10% to less than about 30%, about 10% to less than about 25%, about 10% to less than about 20%, about 10% to less than about 15%) of the tumor sample, for example, by area.

In some instances, the tumor sample from the subject has been determined to have a detectable expression level of PD-L1 in greater than, or equal to, 1% and less than 5% of the tumor cells in the tumor sample. In some instances, the tumor sample from the subject has been determined to have a detectable expression level of PD-L1 in greater than, or equal to, 5% and less than 50% of the tumor cells in the tumor sample. In some instances, the tumor sample from the subject has been determined to have a detectable expression level of PD-L1 in greater than, or equal to, 50% of the tumor cells in the tumor sample.

In some instances, the tumor sample from the subject has been determined to have a detectable expression level of PD-L1 in tumor-infiltrating immune cells that comprise greater than, or equal to, 1% (e.g., about 1% or more, about 2% or more, about 3% or more, about 4% or more, about 5% or more, about 10% or more, about 15% or more, about 20% or more, about 25% or more, about 30% or more, about 35% or more, about 40% or more, about 50% or more, about 55% or more, about 60% or more, about 65% or more, about 70% or more, about 80% or more, about 85% or more, about 90% or more, about 95% or more, or about 99% or more) of the tumor sample, for example, by area. For example, in some instances, the tumor sample has a detectable expression level of PD-L1 in tumor-infiltrating immune cells that comprise about 1% to less than about 99% (e.g., about 1% to less than about 95%, about 1% to less than about 90%, about 1% to less than about 85%, about 1% to less than about 80%, about 1% to less than about 75%, about 1% to less than about 70%, about 1% to less than about 65%, about 1% to less than about 60%, about 1% to less than about 55%, about 1% to less than about 50%, about 1% to less than about 40%, about 1% to less than about 35%, about 1% to less than about 30%, about 1% to less than about 25%, about 1% to less than about 20%, about 1% to less than about 15%, about 1% to less than about 10%, about 1% to less than about 5%, about 5% to less than about 95%, about 5% to less than about 90%, about 5% to less than about 85%, about 5% to less than about 80%, about 5% to less than about 75%, about 5% to less than about 70%, about 5% to less than about 65%, about 5% to less than about 60%, about 5% to less than about 55%, about 5% to less than about 50%, about 5% to less than about 40%, about 5% to less than about 35%, about 5% to less than about 30%, about 5% to less than about 25%, about 5% to less than about 20%, about 5% to less than about 15%, about 5% to less than about 10%, about 10% to less than about 95%, about 10% to less than about 90%, about 10% to less than about 85%, about 10% to less than about 80%, about 10% to less than about 75%, about 10% to less than about 70%, about 10% to less than about 65%, about 10% to less than about 60%, about 10% to less than about 55%, about 10% to less than about 50%, about 10% to less than about 40%, about 10% to less than about 35%, about 10% to less than about 30%, about 10% to less than about 25%, about 10% to less than about 20%, about 10% to less than about 15%) of the tumor sample, for example, by area.

In some instances, the tumor sample from the subject has been determined to have a detectable expression level of PD-L1 in tumor-infiltrating immune cells that comprise greater than, or equal to, 1% and less than 5% of the tumor sample. In some instances, the tumor sample from the subject has been determined to have a detectable expression level of PD-L1 in tumor-infiltrating immune cells that comprise greater than, or equal to, 5% and less than 10% of the tumor sample. In some instances, the tumor sample from the subject has been determined to have a detectable expression level of PD-L1 in tumor-infiltrating immune cells that comprise greater than, or equal to, 10% of the tumor sample.

In some instances, the expression levels or amount of a biomarker is a detectable nucleic acid expression level of PD-L1 in a tumor sample from the subject. In some instances, the PD-L1 nucleic acid expression level has been determined by RNA-seq, RT-qPCR, qPCR, multiplex qPCR, or RT-qPCR, microarray analysis, serial analysis of gene expression (SAGE), MassARRAY® technique, in situ hybridization (ISH), or a combination thereof.

In some instances, the presence and/or expression levels/amount of the biomarker (e.g., PD-L1) in a sample from a subject selects the subject as eligible for therapy with an anti-TIGIT antagonist antibody and an anti-PD-L1 antagonist antibody, for example, where PD-L1 is a biomarker for selection of individuals. In some instances, the sample is selected from the group consisting of a tissue sample, a whole blood sample, a serum sample, and a plasma sample. In some instances, the tissue sample is a tumor sample. In some instances, the tumor sample comprises tumor-infiltrating immune cells, tumor cells, stromal cells, and any combinations thereof.

In one aspect, the invention provides methods for selecting a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1%, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the method further includes administering to the identified subject the therapy. In another aspect, the invention provides methods for selecting a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1%. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In another aspect, the invention provides methods for selecting a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1% and less than 50%, wherein the anti-TIGIT antagonist antibody comprises: a VH domain comprising the amino acid sequence of SEQ ID NO: 17 or 18, and a VL domain comprising the amino acid sequence of SEQ ID NO: 19. In another aspect, the invention provides methods for selecting a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1% and less than 50. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In another aspect, the invention provides methods for selecting a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 50%, wherein the anti-TIGIT antagonist antibody comprises: a VH domain comprising the amino acid sequence of SEQ ID NO: 17 or 18 and a VL domain comprising the amino acid sequence of SEQ ID NO: 19. In another aspect, the invention provides methods for selecting a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 50%. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for identifying a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who may benefit from a therapy comprising an anti-TIGIT antagonist antibody and an anti-PD-L1 antagonist antibody, by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1%, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the invention provides methods for identifying a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who may benefit from a therapy comprising an anti-TIGIT antagonist antibody and an anti-PD-L1 antagonist antibody, by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1%. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for identifying a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who may benefit from a therapy comprising an anti-TIGIT antagonist antibody and an anti-PD-L1 antagonist antibody, by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1% and less than 50%, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the invention provides methods for identifying a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who may benefit from a therapy comprising an anti-TIGIT antagonist antibody and an anti-PD-L1 antagonist antibody, by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1% and less than 50%. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for identifying a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who may benefit from a therapy comprising an anti-TIGIT antagonist antibody and an anti-PD-L1 antagonist antibody, by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 50%, wherein the anti-TIGIT antagonist antibody comprises: a VH domain comprising the amino acid sequence of SEQ ID NO: 17 or 18 and a VL domain comprising the amino acid sequence of SEQ ID NO: 19. In some instances, the invention provides methods for identifying a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who may benefit from a therapy comprising an anti-TIGIT antagonist antibody and an anti-PD-L1 antagonist antibody, by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 50%. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for assessing responsiveness of a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) to a therapy comprising an anti-TIGIT antagonist antibody and an anti-PD-L1 antagonist antibody, by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1%, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the invention provides methods for assessing responsiveness of a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) to a therapy comprising an anti-TIGIT antagonist antibody and an anti-PD-L1 antagonist antibody, by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1%. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for assessing responsiveness of a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) to a therapy comprising an anti-TIGIT antagonist antibody and an anti-PD-L1 antagonist antibody, by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1% and less than 50%, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the invention provides methods for assessing responsiveness of a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) to a therapy comprising an anti-TIGIT antagonist antibody and an anti-PD-L1 antagonist antibody, by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1% and less than 50%. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods assessing responsiveness of a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) to a therapy comprising an anti-TIGIT antagonist antibody and an anti-PD-L1 antagonist antibody, by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 50%, wherein the anti-TIGIT antagonist antibody comprises: a VH domain comprising the amino acid sequence of SEQ ID NO: 17 or 18 and a VL domain comprising the amino acid sequence of SEQ ID NO: 19. In some instances, the invention provides methods assessing responsiveness of a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) to a therapy comprising an anti-TIGIT antagonist antibody and an anti-PD-L1 antagonist antibody, by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 50%. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for optimizing a therapy comprising an anti-TIGIT antagonist antibody and an anti-PD-L1 antagonist antibody in a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1%, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the invention provides methods for optimizing a therapy comprising an anti-TIGIT antagonist antibody and an anti-PD-L1 antagonist antibody in a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1%. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods for optimizing a therapy comprising an anti-TIGIT antagonist antibody and an anti-PD-L1 antagonist antibody in a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1% and less than 50%, wherein the anti-TIGIT antagonist antibody comprises: a VH domain comprising the amino acid sequence of SEQ ID NO: 17 or 18, and a VL domain comprising the amino acid sequence of SEQ ID NO: 19. In some instances, the invention provides methods for optimizing a therapy comprising an anti-TIGIT antagonist antibody and an anti-PD-L1 antagonist antibody in a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1% and less than 50%. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, the invention provides methods optimizing a therapy comprising an anti-TIGIT antagonist antibody and an anti-PD-L1 antagonist antibody in a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 50%, wherein the anti-TIGIT antagonist antibody comprises: a VH domain comprising the amino acid sequence of SEQ ID NO: 17 or 18 and a VL domain comprising the amino acid sequence of SEQ ID NO: 19. In some instances, the invention provides methods optimizing a therapy comprising an anti-TIGIT antagonist antibody and an anti-PD-L1 antagonist antibody in a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by obtaining a tumor sample from the subject, detecting the protein expression level of PD-L1 in the tumor sample by an IHC assay using anti-PD-L1 antibody 22C3 and determining a TPS therefrom, and identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 50%. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

Additionally provided herein are methods for selecting a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein therapy is guided by diagnostic methods that involve detecting the mutational status of EGFR and ALK in a sample obtained from the subject.

In some instances, the method includes detecting the mutational status of EGFR and ALK in a sample from the subject and detecting the absence of a sensitizing EGFR gene mutation or ALK gene rearrangement, and selecting for the subject a therapy comprising one or more dosing cycles of an anti-TIGIT antagonist antibody (e.g., an anti-TIGIT antagonist antibody disclosed herein, e.g., tiragolumab) at a fixed dose of between about 30 mg to about 1200 mg every three weeks and one or more dosing cycles of an anti-PD-L1 antagonist antibody, e.g., atezolizumab) at a fixed dose of between about 80 mg to about 1600 mg every three weeks, based on the subject not having a sensitizing EGFR gene mutation or ALK gene rearrangement. In some instances, the method includes detecting the mutational status of EGFR and ALK in a sample from the subject and detecting the absence of a sensitizing EGFR gene mutation or ALK gene rearrangement, and selecting for the subject a therapy comprising one or more dosing cycles of an anti-TIGIT antagonist antibody (e.g., an anti-TIGIT antagonist antibody disclosed herein, e.g., tiragolumab) at a fixed dose of about 600 mg every three weeks and one or more dosing cycles of an anti-PD-L1 antagonist antibody (e.g., atezolizumab) ata fixed dose of 1200 mg every three weeks, based on the subject not having a sensitizing EGFR gene mutation or ALK gene rearrangement.

Methods for detecting the mutational status EGFR and ALK are well known in the art, and include, but are not limited to, sequencing DNA from clinical samples (e.g., tumor biopsies or blood samples (e.g., circulating tumor DNA in blood)) using a next-generation sequencing method, such as the targeted gene pulldown and sequencing method described in Frampton et al. (Nature Biotechnology. 31(11): 1023-1033, 2013), which is incorporated by reference herein in its entirety. Such a next-generation sequencing method can be used with any of the methods disclosed herein to detect various mutations (e.g., insertions, deletions, base substitutions, focal gene amplifications, and/or homozygous gene deletions), while enabling the use of small samples (e.g., from small-core needle biopsies, fine-needle aspirations, and/or cell blocks) or fixed samples (e.g., formalin-fixed and paraffin-embedded (FFPE) samples). Other methods for the detection of the mutational status of EGFR and ALK include fluorescence in situ hybridization (FISH) and immunohistochemical (IHC) methods. Exemplary methods for the detection of the mutational status of ALK are disclosed in U.S. Pat. No. 9,651,555, which is herein incorporated by reference in its entirety. In some instances, the VENTANA® anti-ALK(D5F3) IHC assay is used to determine the mutational status of the ALK gene.

In some instances, the mutational status of EGFR and ALK in a sample from a subject is used to identify or select the subject as eligible for therapy comprising one or more dosing cycles of an anti-TIGIT antagonist antibody and an anti-PD-L1 antagonist antibody (e.g., an anti-TIGIT antagonist antibody disclosed herein, e.g., tiragolumab) at a fixed dose of between about 30 mg to about 1200 mg every three weeks and one or more dosing cycles of an anti-PD-L1 antagonist antibody, e.g., atezolizumab) at a fixed dose of between about 80 mg to about 1600 mg every three weeks (e.g., where the absence of a sensitizing EGFR gene mutation or ALK gene rearrangement may be used for identification or selection of individuals who are candidates for the therapy comprising one or more dosing cycles of an anti-TIGIT antagonist antibody and an anti-PD-L1 antagonist antibody as described herein. In some instances, the sample is selected from the group consisting of a tissue sample, a whole blood sample, a serum sample, and a plasma sample. In some instances, the tissue sample is a tumor sample.

In one aspect, the invention provides a method for selecting a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by detecting the mutational status of the EGFR gene and ALK gene from a sample from the subject and detecting the absence of a sensitizing EGFR gene mutation or ALK gene rearrangement; and selecting for the subject a therapy comprising one or more dosing cycles of an anti-TIGIT antagonist antibody administered at a fixed dose of 600 mg every three weeks and atezolizumab administered at a fixed dose of 1200 mg every three weeks, based on the subject not having a sensitizing EGFR gene mutation or ALK gene rearrangement, wherein the anti-TIGIT antagonist antibody comprises a VH domain comprising the amino acid sequence of SEQ ID NO: 17 or 18 and a VL domain comprising the amino acid sequence of SEQ ID NO:19. In another aspect, the invention provides a method for selecting a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by detecting the mutational status of the EGFR gene and ALK gene from a sample from the subject and detecting the absence of a sensitizing EGFR gene mutation or ALK gene rearrangement; and selecting for the subject a therapy comprising one or more dosing cycles of tiragolumab administered at a fixed dose of 600 mg every three weeks and atezolizumab administered at a fixed dose of 1200 mg every three weeks, based on the subject not having a sensitizing EGFR gene mutation or ALK gene rearrangement. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances of any of the methods described herein, the mutation is a sensitizing EGFR mutation. Sensitizing EGFR mutations are well known in the art and include those described in U.S. Publication No: US 2018/0235968 and in Juan et al. (Therapeutic Advances in Medical Oncology. 9(3): 201-216, 2017), which are incorporated by reference herein in their entireties. In some instances, the sensitizing EGFR mutation is a mutation in any one of exons 18-21 (e.g., a mutation in exon 18, exon 19, exon 20, and/or exon 21). In some instances, the sensitizing EGFR mutation is a deletion of exon 19 (dell 9). In other instances, sensitizing EGFR mutation is a L858R point mutation in exon 21. In some instances, the sensitizing EGFR mutation is a G719X point mutation in exon 18, wherein “X” is most commonly C, A, or S. In some instances, the sensitizing EGFR mutation is a G719S point mutation in exon 18. In some instances, the sensitizing EGFR mutation is a G719A point mutation in exon 18. In some instances, the sensitizing EGFR mutation is a S720F point mutation in exon 18. In some instances, the sensitizing EGFR mutation is a L861Q point mutation in exon 21. In some instances, the sensitizing EGFR mutation is a L861R point mutation in exon 21. In other instances, the sensitizing EGFR mutation is a T790M point mutation. In some instances, the sensitizing EGFR mutation is an E709X point mutation, where “X” is most commonly K, A, or H. In some instances, the sensitizing EGFR mutation is a S7681 point mutation.

In some instances of any of the methods described herein, the mutation is an ALK gene rearrangement. ALK gene rearrangements are well known in the art and include those described in U.S. Pat. No. 9,651,555 and in Du et al. (Thoracic Cancer. 9: 423-430, 2018), which are incorporated herein by reference in their entireties. In some instances, the ALK gene rearrangement results in the creation of an oncogenic ALK tyrosine kinase that activates downstream signaling pathways resulting in increased cell proliferation and survival. In some instances, the ALK gene rearrangement is an ALK rearrangement with a gene selected from the group consisting of EML4, KIF5B, KLC1, TFG, TPR, HIP1, STRN, DCTN1, SQSTM1, NPM1, BCL11A, B/RC6, RANBP2, ATIC, CLTC, TMP4, and MSN resulting in the formation of a fusion oncogene. In some instances, the ALK gene rearrangement is an EML4 rearrangement with ALK resulting in the formation of the fusion oncogene EML4-ALK.

Additionally provided herein are methods for selecting a therapy for a subject having a non-small cell lung cancer (NSCLC) (e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein therapy is guided by diagnostic methods that involve detecting the subtype of NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) in a sample obtained from the subject.

In some instances, the method includes detecting a subtype of NSCLC other than a pulmonary lymphoepithelioma-like carcinoma subtype of NSCLC in a sample from the subject, and selecting for the subject a therapy comprising one or more dosing cycles of an anti-TIGIT antagonist antibody (e.g., an anti-TIGIT antagonist antibody disclosed herein, e.g., tiragolumab) at a fixed dose of between about 30 mg to about 1200 mg every three weeks and one or more dosing cycles of an anti-PD-L1 antagonist antibody, e.g., atezolizumab) at a fixed dose of between about 80 mg to about 1600 mg every three weeks, based on the subject not having a pulmonary lymphoepithelioma-like carcinoma subtype of NSCLC. In some instances, the method includes detecting a subtype of NSCLC other than a pulmonary lymphoepithelioma-like carcinoma subtype of NSCLC, and selecting for the subject a therapy comprising one or more dosing cycles of an anti-TIGIT antagonist antibody (e.g., an anti-TIGIT antagonist antibody disclosed herein, e.g., tiragolumab) at a fixed dose of about 600 mg every three weeks and one or more dosing cycles of an anti-PD-L1 antagonist antibody (e.g., atezolizumab) at a fixed dose of 1200 mg every three weeks, based on the subject not having a pulmonary lymphoepithelioma-like carcinoma subtype of NSCLC.

Methods for detecting the subtype of NSCLC are well known in the art, and include, but are not limited to, methods of determination by histopathological criteria, or by molecular features (e.g., a subtype characterized by expression of one or a combination of biomarkers (e.g., particular genes or proteins encoded by said genes)). In some instances, the sample is selected from the group consisting of a tissue sample, a whole blood sample, a serum sample, and a plasma sample. In some instances, the tissue sample is a tumor sample.

In some instances, the subtype of NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) determined from the sample obtained from the subject is used to identify or select the subject as eligible for therapy comprising one or more dosing cycles of an anti-TIGIT antagonist antibody and an anti-PD-L1 antagonist antibody (e.g., an anti-TIGIT antagonist antibody disclosed herein, e.g., tiragolumab) at a fixed dose of between about 30 mg to about 1200 mg every three weeks and one or more dosing cycles of an anti-PD-L1 antagonist antibody (e.g., atezolizumab) at a fixed dose of between about 80 mg to about 1600 mg every three weeks (e.g., where the absence of a pulmonary lymphoepithelioma-like carcinoma subtype of NSCLC may be used for identification or selection of individuals who are candidates for the therapy comprising one or more dosing cycles of an anti-TIGIT antagonist antibody and an anti-PD-L1 antagonist antibody as described herein.

In one aspect, the invention provides a method for selecting a therapy for a subject having a NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) by detecting a subtype of the NSCLC other than a pulmonary lymphoepithelioma-like carcinoma; and selecting for the subject a therapy comprising one or more dosing cycles of an anti-TIGIT antagonist antibody administered at a fixed dose of 600 mg every three weeks and atezolizumab administered at a fixed dose of 1200 mg every three weeks, based on the subject not having a pulmonary lymphoepithelioma-like carcinoma subtype of NSCLC, wherein the anti-TIGIT antagonist antibody comprises a VH domain comprising the amino acid sequence of SEQ ID NO: 17 or 18 and a VL domain comprising the amino acid sequence of SEQ ID NO: 19. In another aspect, the invention provides a method for selecting a therapy for a subject having a NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) by biopsying a tumor sample from the subject and detecting a subtype of the NSCLC other than a pulmonary lymphoepithelioma-like carcinoma; and selecting for the subject a therapy comprising one or more dosing cycles of an anti-TIGIT antagonist antibody administered at a fixed dose of 600 mg every three weeks and atezolizumab administered at a fixed dose of 1200 mg every three weeks, based on the subject not having a pulmonary lymphoepithelioma-like carcinoma subtype of NSCLC, wherein the anti-TIGIT antagonist antibody comprises a VH domain comprising the amino acid sequence of SEQ ID NO: 17 or 18 and a VL domain comprising the amino acid sequence of SEQ ID NO: 19. In one aspect, the invention provides a method for selecting a therapy for a subject having a NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) by detecting a subtype of the NSCLC other than a pulmonary lymphoepithelioma-like carcinoma; and selecting for the subject a therapy comprising one or more dosing cycles of tiragolumab administered at a fixed dose of 600 mg every three weeks and atezolizumab administered at a fixed dose of 1200 mg every three weeks, based on the subject not having a pulmonary lymphoepithelioma-like carcinoma subtype of NSCLC. In another aspect, the invention provides a method for selecting a therapy for a subject having a NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) by biopsying a tumor sample from the subject and detecting a subtype of the NSCLC other than a pulmonary lymphoepithelioma-like carcinoma; and selecting for the subject a therapy comprising one or more dosing cycles of tiragolumab administered at a fixed dose of 600 mg every three weeks and atezolizumab administered at a fixed dose of 1200 mg every three weeks, based on the subject not having a pulmonary lymphoepithelioma-like carcinoma subtype of NSCLC. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

Additionally provided herein are methods for selecting a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), wherein therapy is guided by diagnostic methods that involve detecting the presence of one or more indicators of active or chronic active EBV infection in a sample obtained from the subject.

Indicators of active or chronic active EBV infections for use in the methods described herein can include, but are not limited to, EBV IgM, EBV IgG, Epstein-Barr nuclear antigen (EBNA), and Epstein-Barr viral particles detected in a sample from the subject (e.g., a blood or serum sample).

In some instances, the method includes detecting the presence of one or more indicators of active or chronic active EBV infection, including EBV IgM, EBV IgG, Epstein-Barr nuclear antigen (EBNA), and Epstein-Barr viral particles in a sample from the subject, and selecting for the subject a therapy comprising one or more dosing cycles of an anti-TIGIT antagonist antibody (e.g., an anti-TIGIT antagonist antibody disclosed herein, e.g., tiragolumab) at a fixed dose of between about 30 mg to about 1200 mg every three weeks and one or more dosing cycles of an anti-PD-L1 antagonist antibody (e.g., atezolizumab) at a fixed dose of between about 80 mg to about 1600 mg every three weeks, based on the subject being (a) negative for EBV IgG and/or EBNA, (b) positive for EBV IgG and/or EBNA, and negative for both EBV IgM and Epstein-Barr viral particles, or (c) negative for EBV IgG, EBV IgM, EBNA, and Epstein-Barr viral particles. In some instances, the method includes detecting the presence of one or more indicators of active or chronic active EBV infection, including EBV IgM, EBV IgG, Epstein-Barr nuclear antigen (EBNA), and Epstein-Barr viral particles in a sample from the subject, and selecting for the subject a therapy comprising one or more dosing cycles of an anti-TIGIT antagonist antibody (e.g., an anti-TIGIT antagonist antibody disclosed herein, e.g., tiragolumab) at a fixed dose of about 600 mg every three weeks and one or more dosing cycles of an anti-PD-L1 antagonist antibody (e.g., atezolizumab) at a fixed dose of 1200 mg every three weeks, based on the subject being (a) negative for EBV IgG and/or EBNA, (b) positive for EBV IgG and/or EBNA, and negative for both EBV IgM and Epstein-Barr viral particles, or (c) negative for EBV IgG, EBV IgM, EBNA, and Epstein-Barr viral particles.

Methods for detecting the presence of one or more indicators of active or chronic active EBV infection, including EBV IgM, EBV IgG, Epstein-Barr nuclear antigen (EBNA), and Epstein-Barr viral particles in a sample from a subject are well known in the art, and include, but are not limited to, methods involving serological diagnosis (e.g., the detection of EBV DNA (e.g., by PCR analysis of a blood sample for the detection of EBV viral particles) or EBV antigens or anti-EBV antibodies (e.g., detection of EBNA, EBV IgM, or EBV IgG using heterophilic antibodies). In some instances, the sample is selected from the group consisting of a whole blood sample, a serum sample, and a plasma sample.

In some instances, the presence or absence of the one or more indicators of active or chronic active EBV infection in a sample from a subject is used to identify or select the subject as eligible for therapy comprising one or more dosing cycles of an anti-TIGIT antagonist antibody and an anti-PD-L1 antagonist antibody (e.g., an anti-TIGIT antagonist antibody disclosed herein, e.g., tiragolumab) at a fixed dose of between about 30 mg to about 1200 mg every three weeks and one or more dosing cycles of an anti-PD-L1 antagonist antibody (e.g., atezolizumab) at a fixed dose of between about 80 mg to about 1600 mg every three weeks (e.g., where the subject is (a) negative for EBV IgG and/or EBNA, (b) positive for EBV IgG and/or EBNA, and negative for both EBV IgM and Epstein-Barr viral particles, or (c) negative for EBV IgG, EBV IgM, EBNA, and Epstein-Barr viral particles, and is identified or selected as a candidate for the therapy comprising one or more dosing cycles of an anti-TIGIT antagonist antibody and an anti-PD-L1 antagonist antibody as described herein.

In one aspect, the invention provides a method for selecting a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by detecting the presence of one or more of Epstein-Barr virus (EBV) IgM, EBV IgG, Epstein-Barr nuclear antigen (EBNA), and Epstein-Barr viral particles in a sample from the subject, and selecting for the subject a therapy comprising one or more dosing cycles of an anti-TIGIT antagonist antibody administered at a fixed dose of 600 mg every three weeks and atezolizumab administered at a fixed dose of 1200 mg every three weeks, on based on the subject being: (a) negative for EBV IgG and/or EBNA, (b) positive for EBV IgG and/or EBNA, and negative for both EBV IgM and Epstein-Barr viral particles, or (c) negative for EBV IgG, EBV IgM, EBNA, and Epstein-Barr viral particles, wherein the anti-TIGIT antagonist antibody comprises a VH domain comprising the amino acid sequence of SEQ ID NO: 17 or 18 and a VL domain comprising the amino acid sequence of SEQ ID NO: 19. In another aspect, the invention provides a method for selecting a therapy for a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) by detecting the presence of one or more of Epstein-Barr virus (EBV) IgM, EBV IgG, Epstein-Barr nuclear antigen (EBNA), and Epstein-Barr viral particles in a sample from the subject, and selecting for the subject a therapy comprising one or more dosing cycles of tiragolumab administered at a fixed dose of 600 mg every three weeks and atezolizumab administered at a fixed dose of 1200 mg every three weeks, on based on the subject being: (a) negative for EBV IgG and/or EBNA, (b) positive for EBV IgG and/or EBNA, and negative for both EBV IgM and Epstein-Barr viral particles, or (c) negative for EBV IgG, EBV IgM, EBNA, and Epstein-Barr viral particles. In some instances, the method further includes administering to the identified subject the therapy. In some instances, the therapy may further include, or be administered in conjunction with (either separately or together), one or more additional anti-cancer therapeutic agent(s) (e.g., an immunomodulatory agent (e.g., an agent that decreases or inhibits one or more immune co-inhibitory receptors (e.g., one or more immune co-inhibitory receptors selected from TIGIT, PD-L1, PD-1, CTLA-4, LAG3, TIM3, BTLA, and/or VISTA), such as a CTLA-4 antagonist, e.g., an anti-CTLA-4 antagonist antibody (e.g., ipilimumab (YERVOY®)), or an agent that increases or activates one or more immune co-stimulatory receptors (e.g., one or more immune co-stimulatory receptors selected from CD226, OX-40, CD28, CD27, CD137, HVEM, and/or GITR), such as an OX-40 agonist, e.g., an OX-40 agonist antibody), a chemotherapeutic agent, a cytotoxic agent, a growth inhibitory agent, a radiotherapy/radiation therapy, and/or an anti-hormonal agent, such as those recited herein above).

In some instances, in any of the diagnostic methods or uses described herein, the cancer is a lung cancer. In some instances, the lung cancer is a NSCLC. The cancer may be at an early or late stage. In some instances, the NSCLC is a squamous NSCLC. In some instances, the NSCLC is a non-squamous NSCLC. In some instances, the NSCLC is a locally advanced unresectable NSCLC. In some instances, the NSCLC is a Stage IIIB NSCLC. In some instances, the NSCLC is a recurrent or metastatic NSCLC. In some instances, the NSCLC is a Stage IV NSCLC. In some instances, the subject has not been previously treated for Stage IV NSCLC.

In some instances, in any of the diagnostic methods or uses described herein, the subject does not have a sensitizing epidermal growth factor receptor (EGFR) gene mutation or anaplastic lymphoma kinase (ALK) gene rearrangement. In some instances, the subject has an Eastern Cooperative Oncology Group (ECOG) Performance Status (PS) of 0 or 1.

In some instances, in any of the diagnostic methods or uses described herein, the subject does not have a pulmonary lymphoepithelioma-like carcinoma subtype of NSCLC.

In some instances, in any of the diagnostic methods or uses described herein, the subject does not have an active EBV infection or a known or suspected chronic active EBV infection. In some instances, the subject is negative for EBV IgM and/or negative by EBV PCR. In some instances, the subject is negative for EBV IgM and/or negative by EBV PCR and is positive for EBV IgG and/or positive for EBNA. In other instances, the subject is negative for EBV IgG and/or negative for EBNA.

### V. Exemplary Antibodies for Use in the Methods and Uses of the Invention

Exemplary anti-TIGIT antagonist antibodies and anti-PD-L1 antagonist antibodies useful for treating a subject (e.g., a human) having cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) in accordance with the methods, uses, and compositions for use of the invention are described herein.

A. Exemplary Anti-TIGIT Antagonist Antibodies

The invention provides anti-TIGIT antagonist antibodies useful for treating cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) in a subject (e.g., a human).

In some instances, the anti-TIGIT antagonist antibody is tiragolumab (CAS Registry Number: 1918185-84-8). Tiragolumab (Genentech) is also known as MTIG7192A.

In certain instances, the anti-TIGIT antagonist antibodies includes at least one, two, three, four, five, or six HVRs selected from: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and/or (f) an HVR-L3 comprising the amino acid sequence of QQYYSTPFT (SEQ ID NO: 6), or a combination of one or more of the above HVRs and one or more variants thereof having at least about 90% sequence identity (e.g., 90%, 91%, 92%, 93%, 94%, 95%, 96%, 97%, 98%, or 99% identity) to any one of SEQ ID NOs: 1-6.

In some instances, any of the above anti-TIGIT antagonist antibodies includes (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4); (e) an HVR-L2 comprising the amino acid sequence of WASTRES (SEQ ID NO: 5); and (f) an HVR-L3 comprising the amino acid sequence of QQYYSTPFT (SEQ ID NO: 6). In some instances, the anti-TIGIT antagonist antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of, SEQ ID NO: 17 or 18 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19. In some instances, the anti-TIGIT antagonist antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of, SEQ ID NO: 17 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19. In some instances, the anti-TIGIT antagonist antibody has a VH domain comprising an amino acid sequence having at least at least 90% sequence identity (e.g., at least 91%, 92%, 93%, 94%, 95%, 96%, 97%, 98%, or 99% sequence identity) to, or the sequence of, SEQ ID NO: 18 and/or a VL domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 19.

In some instances, the anti-TIGIT antagonist antibody further comprises at least one, two, three, or four of the following light chain variable region framework regions (FRs): an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and/or an FR-L4 comprising the amino acid sequence of FGPGTKVEIK (SEQ ID NO: 10), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 7-10. In some instances, for example, the antibody further comprises an FR-L1 comprising the amino acid sequence of DIVMTQSPDSLAVSLGERATINC (SEQ ID NO: 7); an FR-L2 comprising the amino acid sequence of WYQQKPGQPPNLLIY (SEQ ID NO: 8); an FR-L3 comprising the amino acid sequence of GVPDRFSGSGSGTDFTLTISSLQAEDVAVYYC (SEQ ID NO: 9); and an FR-L4 comprising the amino acid sequence of FGPGTKVEIK (SEQ ID NO: 10).

In some instances, the anti-TIGIT antagonist antibody further comprises at least one, two, three, or four of the following heavy chain variable region FRs: an FR-H1 comprising the amino acid sequence of X1VQLQQSGPGLVKPSQTLSLTCAISGDSVS (SEQ ID NO: 11), wherein X₁ is Q or E; an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 11-14. The anti-TIGIT antagonist antibody may further include, for example, at least one, two, three, or four of the following heavy chain variable region FRs: an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-15. In some instances, the anti-TIGIT antagonist antibody includes an FR-H1 comprising the amino acid sequence of EVQLQQSGPGLVKPSQTLSLTCAISGDSVS (SEQ ID NO: 15); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14. In another instance, for example, the anti-TIGIT antagonist antibody may further include at least one, two, three, or four of the following heavy chain variable region FRs: an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and/or an FR-H4 comprising the amino acid sequence of WGQGTLVTVSS (SEQ ID NO: 14), or a combination of one or more of the above FRs and one or more variants thereof having at least about 90% sequence identity (e.g., 90%, 91%, 92%, 93%, 94%, 95%, 96%, 97%, 98%, or 99% identity) to any one of SEQ ID NOs: 12-14 and 16. In some instances, the anti-TIGIT antagonist antibody includes an FR-H1 comprising the amino acid sequence of QVQLQQSGPGLVKPSQTLSLTCAISGDSVS (SEQ ID NO: 16); an FR-H2 comprising the amino acid sequence of WIRQSPSRGLEWLG (SEQ ID NO: 12); an FR-H3 comprising the amino acid sequence of RITINPDTSKNQFSLQLNSVTPEDTAVFYCTR (SEQ ID NO: 13); and an FR-H4 comprising the amino acid sequence of WGQGTLVTVSS (SEQ ID NO: 14).

In another aspect, an anti-TIGIT antagonist antibody is provided, wherein the antibody comprises a VH as in any of the instances provided above, and a VL as in any of the instances provided above, wherein one or both of the variable domain sequences include post-translational modifications.

In some instances, any one of the anti-TIGIT antagonist antibodies described above is capable of binding to rabbit TIGIT, in addition to human TIGIT. In some instances, any one of the anti-TIGIT antagonist antibodies described above is capable of binding to both human TIGIT and cynomolgus monkey (cyno) TIGIT. In some instances, any one of the anti-TIGIT antagonist antibodies described above is capable of binding to human TIGIT, cyno TIGIT, and rabbit TIGIT. In some instances, any one of the anti-TIGIT antagonist antibodies described above is capable of binding to human TIGIT, cyno TIGIT, and rabbit TIGIT, but not murine TIGIT.

In some instances, the anti-TIGIT antagonist antibody binds human TIGIT with a K_{D} of about 10 nM or lower and cyno TIGIT with a K_{D} of about 10 nM or lower (e.g., binds human TIGIT with a K_{D} of about 0.1 nM to about 1 nM and cyno TIGIT with a K_{D} of about 0.5 nM to about 1 nM, e.g., binds human TIGIT with a K_{D} of about 0.1 nM or lower and cyno TIGIT with a K_{D} of about 0.5 nM or lower).

In some instances, the anti-TIGIT antagonist antibody specifically binds TIGIT and inhibit or block TIGIT interaction with poliovirus receptor (PVR) (e.g., the antagonist antibody inhibits intracellular signaling mediated by TIGIT binding to PVR). In some instances, the antagonist antibody inhibits or blocks binding of human TIGIT to human PVR with an 1050 value of 10 nM or lower (e.g., 1 nM to about 10 nM). In some instances, the antagonist antibody inhibits or blocks binding of cyno TIGIT to cyno PVR with an 1050 value of 50 nM or lower (e.g., 1 nM to about 50 nM, e.g., 1 nM to about 5 nM).

In some instances, the methods or uses described herein may include using or administering an isolated anti-TIGIT antagonist antibody that competes for binding to TIGIT with any of the anti-TIGIT antagonist antibodies described above. For example, the method may include administering an isolated anti-TIGIT antagonist antibody that competes for binding to TIGIT with an anti-TIGIT antagonist antibody having the following six HVRs: (a) an HVR-H1 comprising the amino acid sequence of SNSAAWN (SEQ ID NO: 1); (b) an HVR-H2 comprising the amino acid sequence of KTYYRFKWYSDYAVSVKG (SEQ ID NO: 2); (c) an HVR-H3 comprising the amino acid sequence of ESTTYDLLAGPFDY (SEQ ID NO: 3); (d) an HVR-L1 comprising the amino acid sequence of KSSQTVLYSSNNKKYLA (SEQ ID NO: 4), (e) an HVR-L2 comprising the amino acid sequence of WASTRES (SEQ ID NO: 5); and (f) an HVR-L3 comprising the amino acid sequence of QQYYSTPFT (SEQ ID NO: 6). The methods described herein may also include administering an isolated anti-TIGIT antagonist antibody that binds to the same epitope as an anti-TIGIT antagonist antibody described above.

An anti-TIGIT antagonist antibody according to any of the above instances may be a monoclonal antibody, comprising a chimeric, humanized, or human antibody. In some instances, the anti-TIGIT antagonist antibody is tiragolumab. In one instance, an anti-TIGIT antagonist antibody is an antibody fragment, for example, a Fv, Fab, Fab′, scFv, diabody, or F(ab′)₂ fragment. In another instance, the antibody is a full-length antibody, e.g., an intact IgG antibody (e.g., an intact IgG1 antibody) or other antibody class or isotype as defined herein.

In a further aspect, an anti-TIGIT antagonist antibody according to any of the above instances may incorporate any of the features, singly or in combination, as described in Sections 1-6 below.

B. Exemplary Anti-PD-L1 Antagonist Antibodies

Provided herein are methods for treating cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) in a subject (e.g., a human) in a subject comprising administering to the subject an effective amount of an anti-PD-L1 antagonist antibody.

In some instances, the anti-PD-L1 antagonist antibody inhibits the binding of PD-L1 to its binding partners. In a specific aspect, PD-L1 binding partners are PD-1 and/or B7-1. In some instances, the anti-PD-L1 antagonist antibody is capable of inhibiting binding between PD-L1 and PD-1 and/or between PD-L1 and B7-1.

In some particular instances, the anti-PD-L1 antibody is atezolizumab (CAS Registry Number: 1422185-06-5). Atezolizumab (Genentech) is also known as MPDL3280A.

In some instances, the anti-PD-L1 antibody (e.g., atezolizumab) includes at least one, two, three, four, five, or six HVRs selected from: (a) an HVR-H1 sequence is GFTFSDSWIH (SEQ ID NO: 20); (b) an HVR-H2 sequence is AWISPYGGSTYYADSVKG (SEQ ID NO: 21); (c) an HVR-H3 sequence is RHWPGGFDY (SEQ ID NO: 22), (d) an HVR-L1 sequence is RASQDVSTAVA (SEQ ID NO: 23); (e) an HVR-L2 sequence is SASFLYS (SEQ ID NO: 24); and (f) an HVR-L3 sequence is QQYLYHPAT (SEQ ID NO: 25).

In some instances, the anti-PD-L1 antibody (e.g., atezolizumab) comprises a heavy chain and a light chain sequence, wherein: (a) the heavy chain variable (VH) region sequence comprises the amino acid sequence: EVQLVESGGGLVQPGGSLRLSCAASGFTFSDSWIHWVRQAPGKGLEWVAWISPYGGSTYYADSVKGRFTIS ADTSKNTAYLQMNSLRAEDTAVYYCARRHWPGGFDYWGQGTLVTVSS (SEQ ID NO: 26); and (b) the light chain variable (VL) region sequence comprises the amino acid sequence:

(SEQ ID NO: 27)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKR.

In some instances, the anti-PD-L1 antibody (e.g., atezolizumab) comprises a heavy chain and a light chain sequence, wherein:

(a) the heavy chain comprises the amino acid
sequence:
(SEQ ID NO: 28)
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPG;
and
(b) the light chain comprises the amino acid
sequence:
(SEQ ID NO: 29)
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

In some instances, the anti-PD-L1 antibody comprises (a) a VH domain comprising an amino acid sequence comprising having at least 95% sequence identity (e.g., at least 95%, 96%, 97%, 98%, or 99% sequence identity) to, or the sequence of (SEQ ID NO: 26); (b) a VL domain comprising an amino acid sequence comprising having at least 95% sequence identity (e.g., at least 95%, 96%, 97%, 98%, or 99% sequence identity) to, or the sequence of (SEQ ID NO: 27); or (c) a VH domain as in (a) and a VL domain as in (b). In other instances, the anti-PD-L1 antagonist antibody is selected from YW243.55.S70, MDX-1105, and MEDI4736 (durvalumab), and MSB0010718C (avelumab). Antibody YW243.55.S70 is an anti-PD-L1 described in PCT Pub. No. WO 2010/077634. MDX-1105, also known as BMS-936559, is an anti-PD-L1 antibody described in PCT Pub. No. WO 2007/005874. MEDI4736 (durvalumab) is an anti-PD-L1 monoclonal antibody described in PCT Pub. No. WO 2011/066389 and U.S. Pub. No. 2013/034559. Examples of anti-PD-L1 antibodies useful for the methods of this invention, and methods for making thereof are described in PCT Pub. Nos. WO 2010/077634, WO 2007/005874, and WO 2011/066389, and also in U.S. Pat. No. 8,217,149, and U.S. Pub. No. 2013/034559, which are incorporated herein by reference. The anti-PD-L1 antagonist antibodies (e.g., atezolizumab) useful in this invention, including compositions containing such antibodies, may be used in combination with an anti-TIGIT antagonist antibody to treat cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)).

In some instances, the anti-PD-L1 antagonist antibody is a monoclonal antibody. In some instances, the anti-PD-L1 antagonist antibody is an antibody fragment selected from the group consisting of Fab, Fab′-SH, Fv, scFv, and (Fab′)₂ fragments. In some instances, the anti-PD-L1 antagonist antibody is a humanized antibody. In some instances, the anti-PD-L1 antagonist antibody is a human antibody. In some instances, the anti-PD-L1 antagonist antibody described herein binds to human PD-L1.

In a further aspect, an anti-PD-L1 antagonist antibody according to any of the above instances may incorporate any of the features, singly or in combination, as described in Sections 1-6 below.

1. Antibody Affinity

In certain instances, an anti-TIGIT antagonist antibody and/or anti-PD-L1 antagonist antibody provided herein has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M).

In one instance, K_{D} is measured by a radiolabeled antigen binding assay (RIA). In one instance, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 μM or 26 μM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20 ™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another instance, K_{D} is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one instance, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE Evaluation Software version 3.2) by simultaneously fitting the association and dissociation sensorgrams. The equilibrium dissociation constant (K_{D}) is calculated as the ratio k_{off}/kₒₙ. See, for example, Chen et al., J. Mol. Biol. 293:865-881 (1999). If the on-rate exceeds 10⁶M-¹ s-¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain instances, an anti-TIGIT antagonist antibody and/or anti-PD-L1 antagonist antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). Fora review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain instances, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain instances, an anti-TIGIT antagonist antibody and/or anti-PD-L1 antagonist antibody provided herein is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain instances, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some instances, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain instances, an anti-TIGIT antagonist antibody and/or anti-PD-L1 antagonist antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Nati. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Anti-TIGIT antagonist antibody and/or anti-PD-L1 antagonist antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Anti-TIGIT antagonist antibody and/or anti-PD-L1 antagonist antibodies or antibody fragments isolated from human antibody libraries are considered human antibodies or human antibody fragments herein.

6. Antibody Variants

In certain instances, amino acid sequence variants of the anti-TIGIT antagonist antibodies and/or anti-PD-L1 antagonist antibodies of the invention are contemplated. As described in detail herein, anti-TIGIT antagonist antibodies and anti-PD-L1 antagonist antibodies may be optimized based on desired structural and functional properties. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

I. Substitution, Insertion, and Deletion Variants

In certain instances, anti-TIGIT antagonist antibody and/or anti-PD-L1 antagonist antibody variants having one or more amino acid substitutions are provided. Sites of interest for substitutional mutagenesis include the HVRs and FRs. Conservative substitutions are shown in Table 1 under the heading of “preferred substitutions.” More substantial changes are provided in Table 1 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 1
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some instances of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain instances, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain instances of the variant VH and VL sequences provided above, each HVR either is unaltered, or includes no more than one, two, or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

II. Glycosylation Variants

In certain instances, anti-TIGIT antagonist antibodies and/or anti-PD-L1 antagonist antibodies of the invention can be altered to increase or decrease the extent to which the antibody is glycosylated. Addition or deletion of glycosylation sites to anti-TIGIT antagonist antibody and/or anti-PD-L1 antagonist antibody of the invention may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some instances, modifications of the oligosaccharide in an antibody of the invention are made in order to create antibody variants with certain improved properties.

In one instance, anti-TIGIT antagonist antibody and/or anti-PD-L1 antagonist antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Led 3 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In view of the above, in some instances, the methods of the invention involve administering to the subject in the context of a fractionated, dose-escalation dosing regimen an anti-TIGIT antagonist antibody (e.g., an anti-TIGIT antagonist antibody disclosed herein, e.g., tiragolumab) and/or anti-PD-L1 antagonist antibody (e.g., atezolizumab) variant that comprises an aglycosylation site mutation. In some instances, the aglycosylation site mutation reduces effector function of the antibody. In some instances, the aglycosylation site mutation is a substitution mutation. In some instances, the antibody comprises a substitution mutation in the Fc region that reduces effector function. In some instances, the substitution mutation is at amino acid residue N297, L234, L235, and/or D265 (EU numbering). In some instances, the substitution mutation is selected from the group consisting of N297G, N297A, L234A, L235A, D265A, and P329G. In some instances, the substitution mutation is at amino acid residue N297. In a preferred instance, the substitution mutation is N297A.

Anti-TIGIT antagonist antibody and/or anti-PD-L1 antagonist antibody variants are further provided with bisected oligosaccharides, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

III. Fc Region Variants

In certain instances, one or more amino acid modifications are introduced into the Fc region of an anti-TIGIT antagonist (e.g., an anti-TIGIT antagonist antibody disclosed herein, e.g., tiragolumab) antibody and/or anti-PD-L1 antagonist antibody (e.g., atezolizumab) of the invention, thereby generating an Fc region variant (see e.g., US 2012/0251531). The Fc region variant may comprise a human Fc region sequence (e.g., a human IgG1, IgG2, IgG3 or IgG4 Fc region) comprising an amino acid modification (e.g., a substitution) at one or more amino acid positions.

In certain instances, the invention contemplates an anti-TIGIT antagonist antibody and/or anti-PD-L1 antagonist antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII, and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M. S. and M. J. Glennie Blood. 103:2738-2743 (2004)). FcRn binding and in vivo clearance/half-life determinations can also be performed using methods known in the art (see, e.g., Petkova, S. B. et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain instances, the proline at position 329 of a wild-type human Fc region in the antibody is substituted with glycine or arginine or an amino acid residue large enough to destroy the proline sandwich within the Fc/Fc.gamma receptor interface that is formed between the proline 329 of the Fc and tryptophan residues Trp 87 and Trp 110 of FcgRIII (Sondermann et al.: Nature 406, 267-273 (20 Jul. 2000)). In certain instances, the antibody comprises at least one further amino acid substitution. In one instance, the further amino acid substitution is S228P, E233P, L234A, L235A, L235E, N297A, N297D, or P331S, and still in another instance the at least one further amino acid substitution is L234A and L235A of the human IgG1 Fc region or S228P and L235E of the human IgG4 Fc region (see e.g., US 2012/0251531), and still in another instance the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain instance, an antibody variant comprises an Fc region with one or more amino acid substitutions which improve ADCC, e.g., substitutions at positions 298, 333, and/or 334 of the Fc region (EU numbering of residues).

In some instances, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424, or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

In some aspects the anti-TIGIT antagonist antibody (e.g., an anti-TIGIT antagonist antibody disclosed herein, e.g., tiragolumab) and/or anti-PD-L1 antagonist antibody (e.g., atezolizumab) comprises an Fc region comprising an N297G mutation.

In some instances, the anti-TIGIT antagonist antibody (e.g., an anti-TIGIT antagonist antibody disclosed herein, e.g., tiragolumab) and/or anti-PD-L1 antagonist antibody (e.g., atezolizumab) comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain. In some instances, at least one of the one or more heavy chain constant domains is paired with another heavy chain constant domain. In some instances, the CH3₁ and CH3₂ domains each comprise a protuberance or cavity, and wherein the protuberance or cavity in the CH3₁ domain is positionable in the cavity or protuberance, respectively, in the CH3₂ domain. In some instances, the CH3₁ and CH3₂ domains meet at an interface between said protuberance and cavity. In some instances, the CH2₁ and CH2₂ domains each comprise a protuberance or cavity, and wherein the protuberance or cavity in the CH2₁ domain is positionable in the cavity or protuberance, respectively, in the CH2₂ domain. In other instances, the CH2₁ and CH2₂ domains meet at an interface between said protuberance and cavity. In some instances, the anti-TIGIT antagonist antibody (e.g., an anti-TIGIT antagonist antibody disclosed herein, e.g., tiragolumab) and/or anti-PD-L1 antagonist antibody (e.g., atezolizumab) is an IgG1 antibody.

IV. Cysteine Engineered Antibody Variants

In certain instances, it is desirable to create cysteine engineered anti-TIGIT antagonist antibodies and/or anti-PD-L1 antagonist antibodies, e.g., “thioMAbs,” in which one or more residues of an antibody are substituted with cysteine residues. In particular instances, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain instances, any one or more of the following residues are substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

V. Antibody Derivatives

In certain instances, an anti-TIGIT antagonist antibody of the invention (e.g., an anti-TIGIT antagonist antibody (e.g., tiragolumab) or a variant thereof) and/or anti-PD-L1 antagonist antibody of the invention (e.g., atezolizumab or a variant thereof) provided herein are further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another instance, conjugates of an antibody and nonproteinaceous moiety that may be selectively heated by exposure to radiation are provided. In one instance, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

Recombinant Production Methods

Anti-TIGIT antagonist antibodies (e.g., an anti-TIGIT antagonist antibody disclosed herein, e.g., tiragolumab) and/or anti-PD-L1 antagonist antibodies (e.g., atezolizumab) of the invention may be produced using recombinant methods and compositions, for example, as described in U.S. Pat. No. 4,816,567, which is incorporated herein by reference in its entirety.

For recombinant production of an anti-TIGIT antagonist antibody and/or anti-PD-L1 antagonist antibody, nucleic acid encoding an antibody, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Immunoconjugates

The invention also provides immunoconjugates comprising an anti-TIGIT antagonist (e.g., an anti-TIGIT antagonist antibody as disclosed herein, e.g., tiragolumab) and/or anti-PD-L1 antagonist antibody (e.g., atezolizumab) of the invention conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In some instances, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another instance, an immunoconjugate comprises an anti-TIGIT antagonist antibody as described herein (e.g., tiragolumab) or an anti-PD-L1 antagonist antibody (e.g., atezolizumab) conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another instance, an immunoconjugate comprises an anti-TIGIT antagonist antibody as described herein (e.g., tiragolumab) and/or an anti-PD-L1 antagonist antibody as described herein (e.g., atezolizumab) conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker, or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### VI. Pharmaceutical Compositions and Formulations

Any of the anti-TIGIT antagonist antibodies and anti-PD-L1 antagonist antibodies described herein can be used in pharmaceutical compositions and formulations. Pharmaceutical compositions and formulations of an anti-TIGIT antagonist antibody and an anti-PD-L1 antagonist antibody can be prepared by mixing such antibodies having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules. The formulations to be used for in vivo administration are generally sterile. Sterility may be readily accomplished, e.g., by filtration through sterile filtration membranes.

### VII. Articles of Manufacture and Kits

In another aspect of the invention, an article of manufacture or a kit containing materials useful for the treatment, prevention, and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing, and/or diagnosing the condition and may have a sterile access port (for example the container may be an intravenous solution bag or a vial having a stopper pierceable by a hypodermic injection needle).

At least one active agent in the composition is an anti-TIGIT antagonist antibody of the invention (e.g., an anti-TIGIT antagonist antibody as disclosed herein, e.g., tiragolumab). The label or package insert indicates that the composition is used for treating the condition of choice (e.g., cancer, e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)). Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this instance of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution, and dextrose solution. It may further include other materials desirable from a commercial and user standpoint, including other buffers, diluents, filters, needles, and syringes.

In one instance, provided is a kit including an anti-TIGIT antagonist antibody (e.g., an anti-TIGIT antagonist antibody as disclosed herein, e.g., tiragolumab), an anti-PD-L1 antagonist antibody (e.g., atezolizumab), and a package insert comprising instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) the anti-TIGIT antagonist antibody at a fixed dose of between about 30 mg to about 1200 mg every three weeks and the anti-PD-L1 antagonist antibody at a fixed dose of between about 80 mg to about 1600 mg every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a PD-L1 TPS of greater than, or equal to 1%, and less than 50% a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a PD-L1 TPS of greater than, or equal to 50% a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to not have a sensitizing EGFR gene mutation or an ALK gene rearrangement a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a subtype of NSCLC other than pulmonary lymphoepithelioma-like carcinoma a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to be (a) negative for EBV IgG and/or EBNA, (b) positive for EBV IgG and/or EBNA, and negative for both EBV IgM and Epstein-Barr viral particles, or (c) negative for EBV IgG, EBV IgM, EBNA, and Epstein-Barr viral particles a fixed dose of about 600 mg of the anti-TIGIT antagonist antibody every three weeks and a fixed dose of about 1200 mg of the anti-PD-L1 antagonist antibody every three weeks.

In one instance, provided is a kit including tiragolumab, atezolizumab, and a package insert comprising instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) tiragolumab at a fixed dose of between about 30 mg to about 1200 mg every three weeks and atezolizumab at a fixed dose of between about 80 mg to about 1600 mg every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) a fixed dose of about 600 mg of tiragolumab every three weeks and a fixed dose of about 1200 mg of atezolizumab every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a PD-L1 TPS of greater than, or equal to 1%, and less than 50% a fixed dose of about 600 mg of tiragolumab every three weeks and a fixed dose of about 1200 mg of atezolizumab every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a PD-L1 TPS of greater than, or equal to 50% a fixed dose of about 600 mg of tiragolumab every three weeks and a fixed dose of about 1200 mg of atezolizumab every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to not have a sensitizing EGFR gene mutation or an ALK gene rearrangement a fixed dose of about 600 mg of tiragolumab every three weeks and a fixed dose of about 1200 mg of atezolizumab every three weeks. In some instances, the package insert comprises instructions to administer to a subject having a NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a subtype of NSCLC other than pulmonary lymphoepithelioma-like carcinoma a fixed dose of about 600 mg of tiragolumab every three weeks and a fixed dose of about 1200 mg of atezolizumab every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to be (a) negative for EBV IgG and/or EBNA, (b) positive for EBV IgG and/or EBNA, and negative for both EBV IgM and Epstein-Barr viral particles, or (c) negative for EBV IgG, EBV IgM, EBNA, and Epstein-Barr viral particles a fixed dose of about 600 mg of tiragolumab every three weeks and a fixed dose of about 1200 mg of atezolizumab every three weeks.

In some instances, the kit includes an anti-TIGIT antagonist antibody (e.g., an anti-TIGIT antagonist antibody as disclosed herein, e.g., tiragolumab) and a package insert comprising instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) the anti-TIGIT antagonist antibody at a fixed dose of between about 30 mg to about 1200 mg every three weeks and an anti-PD-L1 antagonist antibody at a fixed dose of between about 80 mg to about 1600 mg every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a PD-L1 TPS of greater than, or equal to 1%, and less than 50% a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a PD-L1 TPS of greater than, or equal to 50% a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to not have a sensitizing EGFR gene mutation or an ALK gene rearrangement a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a subtype of NSCLC other than pulmonary lymphoepithelioma-like carcinoma a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to be (a) negative for EBV IgG and/or EBNA, (b) positive for EBV IgG and/or EBNA, and negative for both EBV IgM and Epstein-Barr viral particles, or (c) negative for EBV IgG, EBV IgM, EBNA, and Epstein-Barr viral particles a fixed dose of about 600 mg of the anti-TIGIT antagonist antibody every three weeks and a fixed dose of about 1200 mg of the anti-PD-L1 antagonist antibody every three weeks.

In some instances, the kit includes tiragolumab and a package insert comprising instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) tiragolumab at a fixed dose of between about 30 mg to about 1200 mg every three weeks and atezolizumab at a fixed dose of between about 80 mg to about 1600 mg every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) a fixed dose of about 600 mg of tiragolumab every three weeks and a fixed dose of about 1200 mg of atezolizumab every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a PD-L1 TPS of greater than, or equal to 1%, and less than 50% a fixed dose of about 600 mg of tiragolumab every three weeks and a fixed dose of about 1200 mg of atezolizumab every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a PD-L1 TPS of greater than, or equal to 50% a fixed dose of about 600 mg of tiragolumab every three weeks and a fixed dose of about 1200 mg of atezolizumab every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to not have a sensitizing EGFR gene mutation or an ALK gene rearrangement a fixed dose of about 600 mg of tiragolumab every three weeks and a fixed dose of about 1200 mg of atezolizumab every three weeks. In some instances, the package insert comprises instructions to administer to a subject having a NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a subtype of NSCLC other than pulmonary lymphoepithelioma-like carcinoma a fixed dose of about 600 mg of tiragolumab every three weeks and a fixed dose of about 1200 mg of atezolizumab every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to be (a) negative for EBV IgG and/or EBNA, (b) positive for EBV IgG and/or EBNA, and negative for both EBV IgM and Epstein-Barr viral particles, or (c) negative for EBV IgG, EBV IgM, EBNA, and Epstein-Barr viral particles a fixed dose of about 600 mg of tiragolumab every three weeks and a fixed dose of about 1200 mg of atezolizumab every three weeks.

In some instances, the kit includes an anti-PD-L1 antagonist antibody (e.g., atezolizumab) and a package insert comprising instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) the anti-PD-L1 antagonist antibody at a fixed dose of between about 80 mg to about 1600 mg every three weeks and an anti-TIGIT antagonist antibody at a fixed dose of between about 30 mg to about 1200 mg every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) a fixed dose of about 1200 mg of the anti-PD-L1 antagonist antibody every three weeks and a fixed dose of about 600 mg of the anti-TIGIT antagonist antibody every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a PD-L1 TPS of greater than, or equal to 1%, and less than 50% a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a PD-L1 TPS of greater than, or equal to 50% a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to not have a sensitizing EGFR gene mutation or an ALK gene rearrangement a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a subtype of NSCLC other than pulmonary lymphoepithelioma-like carcinoma a fixed dose of about 600 mg of the anti-TIGIT antagonist antibody every three weeks and a fixed dose of about 1200 mg of the anti-PD-L1 antagonist antibody every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to be (a) negative for EBV IgG and/or EBNA, (b) positive for EBV IgG and/or EBNA, and negative for both EBV IgM and Epstein-Barr viral particles, or (c) negative for EBV IgG, EBV IgM, EBNA, and Epstein-Barr viral particles a fixed dose of about 600 mg of the anti-TIGIT antagonist antibody every three weeks and a fixed dose of about 1200 mg of the anti-PD-L1 antagonist antibody every three weeks.

In some instances, the kit includes atezolizumab and a package insert comprising instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) atezolizumab at a fixed dose of between about 80 mg to about 1600 mg every three weeks and tiragolumab at a fixed dose of between about 30 mg to about 1200 mg every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) a fixed dose of about 1200 mg of atezolizumab every three weeks and a fixed dose of about 600 mg of tiragolumab every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a PD-L1 TPS of greater than, or equal to 1%, and less than 50% a fixed dose of about 600 mg of tiragolumab every three weeks and a fixed dose of about 1200 mg of atezolizumab every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a PD-L1 TPS of greater than, or equal to 50% a fixed dose of about 600 mg of the tiragolumab every three weeks and a fixed dose of about 1200 mg of atezolizumab every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to not have a sensitizing EGFR gene mutation or an ALK gene rearrangement a fixed dose of about 600 mg of tiragolumab every three weeks and a fixed dose of about 1200 mg of atezolizumab every three weeks. In some instances, the package insert comprises instructions to administer to a subject having a NSCLC (e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to have a subtype of NSCLC other than pulmonary lymphoepithelioma-like carcinoma a fixed dose of about 600 mg of tiragolumab every three weeks and a fixed dose of about 1200 mg of atezolizumab every three weeks. In some instances, the package insert comprises instructions to administer to a subject having a cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) who has been determined to be (a) negative for EBV IgG and/or EBNA, (b) positive for EBV IgG and/or EBNA, and negative for both EBV IgM and Epstein-Barr viral particles, or (c) negative for EBV IgG, EBV IgM, EBNA, and Epstein-Barr viral particles a fixed dose of about 600 mg of tiragolumab every three weeks and a fixed dose of about 1200 mg of atezolizumab every three weeks.

In a related instance, the invention features a kit including an anti-TIGIT antagonist antibody (e.g., an anti-TIGIT antagonist antibody disclosed herein, e.g., tiragolumab) of the invention, an anti-PD-L1 antagonist antibody (e.g., atezolizumab) and a package insert comprising instructions for using the anti-TIGIT antagonist antibody and anti-PD-L1 antagonist antibody for treating cancer (e.g., lung cancer, e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)) in a subject according to any of the methods disclosed herein. In any of the above instances, the subject may, for example, be a human. It is specifically contemplated that any of the anti-TIGIT antagonist antibodies and anti-PD-L1 antagonist antibodies described herein may be included in the kit.

### VIII. Example

The following is an example of the methods of the invention. It is understood that various other embodiments may be practiced, given the general description provided above.

### Example 1. Efficacy of an Anti-TIGIT Antagonist Antibody in Combination with an Anti-PD-L1 Antagonist Antibody in Patients with Lung Cancer

To evaluate the efficacy and safety of treatment with an anti-TIGIT antagonist antibody (e.g., an anti-TIGIT antibody disclosed herein, e.g., tiragolumab) in combination with an anti-PD-L1 antagonist antibody (atezolizumab) compared with placebo in combination with atezolizumab in patients with lung cancer (e.g., non-small cell lung cancer (NSCLC), e.g., squamous or non-squamous NSCLC, e.g., locally advanced unresectable NSCLC (e.g., Stage IIIB NSCLC), or recurrent or metastatic NSCLC (e.g., Stage IV NSCLC)), patients are enrolled in a phase II, global, multicenter, randomized, blinded, placebo-controlled study. To be eligible, patients must (i) have not been previously treated for locally advanced unresectable or metastatic NSCLC, (ii) have an Eastern Cooperative Oncology Group (ECOG) Performance Status (PS) of 0 or 1, (iii) have a PD-L1 selected tumor (e.g., a tumor PD-L1 expression with a tumor proportion score (TPS)≥1% as determined by the PD-L1 IHC 22C3 pharmDx assay), (iv) not have an epidermal growth factor receptor (EGFR) or anaplastic lymphoma kinase (ALK) gene mutation, (v) not have a pulmonary lymphoepithelioma-like carcinoma subtype of NSCLC, and (vi) not have an active Epstein-Barr virus (EBV) infection or a known or suspected chronic active EBV infection.

If a patient has positive serology for EBV IgG and/or is positive for Epstein-Barr nuclear antigen (EBNA), then EBV IgM testing and/or EBV PCR is required for consideration of eligibility. If the patient has positive serology for EBV IgG and/or is positive for EBNA, they must be negative for EBV IgM and/or negative by EBV PCR. Additional EBV serology tests are performed for patients who subsequently experience an acute inflammatory event, e.g., systemic inflammatory response syndrome, while receiving study treatment.

The clinical trial consists of a single phase, as described in detail below.

Randomization

In this study, 120 patients are enrolled and randomized to one of two treatment arms in a 1:1 ratio (experimental arm to control arm). In the experimental arm, patients receive an anti-TIGIT antagonist antibody (e.g., an anti-TIGIT antibody disclosed herein, e.g., tiragolumab) in combination with atezolizumab. In the control arm, patients receive a placebo in combination with atezolizumab. The randomization is stratified on the basis of PD-L1 IHC 22C3 pharmDx assay results (e.g., a TPS of between 1-49% versus a TPS of ≥50%), histology of NSCLC (e.g., non-squamous versus squamous), and the patient's history of tobacco use (e.g., yes or no). These stratification factors have been identified as critical prognostic factors for patients with NSCLC. Prospective stratification by these factors will minimize differences in the two treatment arms due to sources other than the anti-TIGIT antagonist antibody.

Study Treatment Dosage and Administration

During treatment, patients receive a fixed dose of 600 mg of an anti-TIGIT antagonist antibody (e.g., an anti-TIGIT antibody disclosed herein, e.g., tiragolumab) or placebo (equivalent to an average body weight-based dose of 7.5 mg/kg) administered by intravenous infusion every 3 weeks (q3w) (21±3 days). The anti-TIGIT antagonist antibody (e.g., an anti-TIGIT antibody disclosed herein, e.g., tiragolumab) or placebo is administered on Day 1 of each 21-day dosing cycle. Atezolizumab is administered by intravenous infusion at a dose of 1200 mg (equivalent to an average body weight-based dose of 15 mg/kg) every 3 weeks (21±3 days). The atezolizumab dose is fixed and is not dependent on body weight. Atezolizumab is administered on Day 1 of each 21-day dosing cycle.

In one experiment of the study, on the days of administration, the anti-TIGIT antagonist antibody (e.g., an anti-TIGIT antibody disclosed herein, e.g., tiragolumab) or placebo is administered prior to atezolizumab, with an intervening observation period. Prior to the first infusion of the anti-TIGIT antibody or placebo, the patient's vital signs (e.g., pulse rate, respiratory rate, blood pressure, and temperature) are recorded within 60 minutes before starting the infusion. The first infusion of the anti-TIGIT antibody (e.g., an anti-TIGIT antibody disclosed herein, e.g., tiragolumab) or placebo is administered over 60 (±10) minutes. During this time, the patient's vital signs (pulse rate, respiratory rate, blood pressure, and temperature) are recorded at 15-minute intervals. Following infusion, the patient is observed for 60 minutes, during which time, the vital signs are monitored as described above. The first infusion of atezolizumab is administered over 60 (±15) minutes. During this time, the patient's vital signs are recorded at 15-minute intervals. Following infusion, the patient is observed for 60 minutes, during which time the vital signs are monitored as described above. If no infusion-associated adverse events are experienced during the first infusions of the anti-TIGIT antagonist antibody (e.g., an anti-TIGIT antibody disclosed herein, e.g., tiragolumab), placebo, or atezolizumab, subsequent infusions can be administered over 30 (±10) minutes. Additionally, the post-infusion observation periods may be reduced to 30 minutes. Pre-infusion recordation of vital signs shall continue to be recorded within 60 minutes prior to the start of infusion of the anti-TIGIT antagonist antibody (e.g., an anti-TIGIT antibody disclosed herein, e.g., tiragolumab) or placebo.

In another experiment of the study, on the days of administration, atezolizumab is administered prior to the anti-TIGIT antagonist antibody (e.g., an anti-TIGIT antibody disclosed herein, e.g., tiragolumab) or placebo, with an intervening observation period. Prior to the first infusion of atezolizumab, the patient's vital signs (e.g., pulse rate, respiratory rate, blood pressure, and temperature) are recorded within 60 minutes before starting the infusion. The first infusion of atezolizumab is administered over 60 (±15) minutes. During this time, the patient's vital signs (pulse rate, respiratory rate, blood pressure, and temperature) are recorded at 15-minute intervals. Following infusion, the patient is observed for 60 minutes, during which time, the vital signs are monitored as described above. The first infusion of the anti-TIGIT antibody (e.g., an anti-TIGIT antibody disclosed herein, e.g., tiragolumab) or placebo is administered over 60 (±10) minutes. During this time, the patient's vital signs are recorded at 15-minute intervals. Following infusion, the patient is observed for 60 minutes, during which time the vital signs are monitored as described above. If no infusion-associated adverse events are experienced during the first infusions of atezolizumab, placebo, or the anti-TIGIT antagonist antibody (e.g., an anti-TIGIT antibody disclosed herein, e.g., tiragolumab), subsequent infusions can be administered over 30 (±10) minutes. Additionally, the post-infusion observation periods may be reduced to 30 minutes. Pre-infusion recordation of vital signs shall continue to be recorded within 30 minutes prior to the start of infusion of atezolizumab.

Treatment is continued until lack of clinical benefit, worsening of symptoms attributed to disease progression following an integrated assessment of radiographic data, biopsy results, and clinical status, decline in performance status, intolerable toxicity related to the study treatment, or tumor progression at a critical site that cannot be managed with protocol-accepted therapy.

Concomitant Therapy

Certain concomitant therapies are permitted. Concomitant therapies include any medication (e.g., prescription drugs, over the counter drugs, vaccines, herbal or homeopathic remedies, nutritional supplements) used by a patient in addition to protocol-mandated study treatment from seven days prior to initiation of study treatment to the treatment discontinuation visit. Patients are permitted to use the following concomitant therapies during the study.

Systemic corticosteroids and other immune-modulating medications may, in theory, attenuate the potential beneficial immunologic effects of treatment with the anti-TIGIT antagonist antibody and/or atezolizumab, but should be administered at the discretion of the treating physician in line with the management guidelines. No premedication is allowed for the first infusion of atezolizumab, the anti-TIGIT antagonist antibody (e.g., an anti-TIGIT antibody disclosed herein, e.g., tiragolumab), or placebo. If the patient experienced an infusion-related reaction (IRR) during any previous infusion of atezolizumab, the anti-TIGIT antagonist antibody (e.g., an anti-TIGIT antibody disclosed herein, e.g., tiragolumab), or placebo, premedication with an antihistamine and/or antipyretic may be administered for Cycles 2 at the discretion of the treating physician after consultation with the medical monitor. The use of inhaled corticosteroids and mineralocorticoids (e.g., fludrocortisone) for patients with orthostatic hypotension or adrenocortical insufficiency is also allowed. Physiologic doses of corticosteroids for adrenal insufficiency are allowed.

Patients with abnormal renal function should be evaluated and treated for other more common etiologies (e.g., prerenal and postrenal causes and concomitant medications including NSAIDs). Renal biopsies may be required to determine a definitive diagnosis and appropriate treatment. Patients presenting with signs and symptoms of nephritis, in the absence of an identified alternate etiology, should be evaluated and treated according to the severity of the event. If the patient presents with a grade 1 renal event, study treatment may continue while kidney functions (e.g., creatinine levels) are monitored and resolve to within normal limits and/or baseline values. Patients experiencing a grade 2 event should have the study treatment withheld for up to twelve weeks and treated with corticosteroids until the resolution of symptoms. Patients may resume the study treatment following a tapering period over at least one month of corticosteroids to an equivalent dose of ≤10 mg/day oral prednisone. Patients experiencing a grade 3 or grade 4 renal event should permanently discontinue treatment with the anti-TIGIT antibody (e.g., tiragolumab)/placebo and atezolizumab and be treated with corticosteroids and/or immunosuppressive agents.

Megestrol administered as an appetite stimulant is acceptable while the patient is enrolled in the study. Patients who use oral contraceptives, hormone-replacement therapy, prophylactic or therapeutic anticoagulation therapy (such as low molecular weight heparin or warfarin at a stable dose level), or other maintenance therapy for non-malignant indications should continue their use. Cannabinoids are permitted only if obtained in accordance with local regulations, and only if an established part of patient management prior to study enrolment.

Certain forms of radiotherapy may be considered for pain palliation if patients are deriving benefit (e.g., treatment of known bony metastases) and provided they do not compromise assessments of tumor target lesions. In addition, the anti-TIGIT antagonist antibody (e.g., an anti-TIGIT antibody disclosed herein, e.g., tiragolumab) or placebo and atezolizumab treatment can continue during palliative radiotherapy. Patients experiencing a mixed response requiring local therapy (e.g., surgery, stereotactic radiosurgery, radiotherapy, radiofrequency ablation) for control of three or fewer lesions may still be eligible to continue study treatment, at the discretion of the investigator, and after discussion with the medical monitor. Subsequent tumor assessments may need to take the local treatment into account in determining overall response per the response evaluation criteria in solid tumors (RECIST) v1.1 or per the immune-modified RECIST (imRECIST) criteria (see, e.g., Hodi et al. J. Clin. Oncol. e-pub, Jan. 17, 2018, which is hereby incorporated by reference in its entirety), as appropriate.

Patients receiving denosumab prior to enrollment are maintained on bisphosphonate therapy instead (if willing and eligible) during screening and while actively treated with study drug. Initiation of bisphosphonates is discouraged during the treatment phase of the study due to potential immunomodulatory properties, however, initiation of such treatment should not result in discontinuation of study treatment.

In some instances, premedication with antihistamines, antipyretics, and/or analgesics are administered for the second and subsequent anti-TIGIT antagonist antibody (e.g., an anti-TIGIT antibody disclosed herein, e.g., tiragolumab) or placebo and atezolizumab infusions only, at the discretion of the investigator. In general, investigators can manage a patient's care with supportive therapies as clinically indicated, per local standard practice. Patients who experience infusion associated symptoms can receive treatment symptomatically with acetaminophen, ibuprofen, diphenhydramine, and/or H2 receptor antagonists (e.g., famotidine, cimetidine), or equivalent medications per local standard practice. Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and β₂ adrenergic agonists).

Efficacy Endpoints

Co-primary and secondary efficacy analyses among all randomized patients are conducted when approximately 80 total progression-free survival (PFS) events occur.

To evaluate the efficacy of the anti-TIGIT antagonist antibody (e.g., an anti-TIGIT antibody disclosed herein, e.g., tiragolumab) in combination with atezolizumab compared with placebo in combination with atezolizumab, the objective response rate (ORR), with ORR defined as the percentage of patients who have experienced a complete response (CR) or a partial response (PR) on two consecutive occasions weeks apart (as determined by the investigator according to RECIST v1.1), is measured as a primary endpoint. The difference in ORR between the two study arms is estimated, along with PFS hazard ratios (HRs) with 90% confidence interval (CI). The ORRs between the two treatment arms are compared at the two-sided significance level of 5% using the Mantel-Haenszel Test, stratified by the study's stratification factors (i.e., PD-L1 IHC 22C3 pharmDx assay results (e.g., a TPS of between 1-49% versus a TPS of 50%), histology of NSCLC (e.g., non-squamous versus squamous), and the patient's history of tobacco use (e.g., yes or no)). An additional primary efficacy endpoint further includes progression-free survival (PFS), defined as the time from randomization to the date of first documented disease progression or death, whichever occurs first. A stratified Cox proportional-hazards model is used to estimate the HR and its 90% CI. PFS between treatment arms is compared using the two-sided stratified log-rank test. Kaplan-Meier methodology is used to estimate a PFS curve and median PFS for each treatment arm.

Secondary efficacy endpoints can include duration of objective response (DOR), defined as the time from the first occurrence of a documented objective response to disease progression (as determined by the investigator according to RECIST v1.1), or death from any cause, whichever occurs first, or overall survival (OS) (i.e., the time from randomization to death from any cause). A stratified Cox proportional-hazards model is used to estimate the HR and its 90% CI. OS between treatment arms is compared using the two-sided stratified log-rank test. Kaplan-Meier methodology is used to estimate an OS curve and median OS for each treatment arm.

Additional exploratory efficacy endpoints may further include evaluating ORR, DOR, and PFS according to immune-modified RECIST (imRECIST) criteria (see, e.g., Hodi et al. J. Clin. Oncol. e-pub, Jan. 17, 2018, which is hereby incorporated by reference in its entirety), which are based on key principles from immune-related response criteria that were originally designed to account for tumor change patterns observed in melanoma patients treated with the CTLA-4 inhibitor ipilimumab (see, e.g., Wolchok et al. Clin. Can. Res. 15(23): 7412-20, 2009, which is hereby incorporated by reference in its entirety).

To evaluate the safety and tolerability of the anti-TIGIT antagonist antibody (e.g., an anti-TIGIT antibody disclosed herein, e.g., tiragolumab) in combination atezolizumab compared with the placebo in combination atezolizumab, the incidence, nature, and severity of adverse events (AEs) (e.g., AEs graded according to the National Cancer Institute Common Terminology Criteria for Adverse Events version 4.0 (NCI CTCAE v4.0)) are measured. Additionally, clinically significant changes in vital signs, physical findings, and clinical laboratory results from baseline during and following administration of the anti-TIGIT antagonist antibody (e.g., an anti-TIGIT antibody disclosed herein, e.g., tiragolumab) in combination with atezolizumab compared with placebo in combination with atezolizumab are also measured as an endpoint. Yet further efficacy endpoints can include changes in health-related quality of life (HRQoL) as assessed by symptoms in lung cancer (SILO) scale (e.g., time to deterioration (TTD) in cough dyspenea and chest pain), the European organization for research and treatment of Cancer (EORTC) quality of life questionnaire C30 (QLC-C-30) (e.g., mean change from baseline in HRQoL and day-to-day function as measured by the global health status, physical function, and role function scales), and the EuroQol 5-Dimension, 5-Level Questionnaire (EQ-5D-5L) questionnaire (e.g., capture utility values) for health economic modeling, and/or tolerability of the anti-TIGIT antagonist antibody (e.g., an anti-TIGIT antibody disclosed herein, e.g., tiragolumab) in combination with atezolizumab or the placebo in combination with atezolizumab.

Biomarkers

Patient samples, including archival tumor tissues, as well as serum, plasma, whole blood, and stool are collected for exploratory biomarker assessments for all patients in the randomized study. In addition to assessing PD-L1 status, biomarkers related to resistance, disease progression, and clinical benefit of the anti-TIGIT antagonist antibody (e.g., an anti-TIGIT antibody disclosed herein, e.g., tiragolumab) and/or atezolizumab are analyzed. For example, potential predictive and prognostic biomarkers related to the clinical benefit and safety of the anti-TIGIT antagonist antibody (e.g., an anti-TIGIT antibody disclosed herein, e.g., tiragolumab) and/or atezolizumab are analyzed.

Tumor tissue and blood samples collected at baseline (and, if deemed clinically feasible by the investigator, tumor tissue collected at the time of disease progression) enables whole-exome sequencing (WES) and/or next-generation sequencing (NGS) to identify somatic mutations that are predictive of response to study treatment, are associated with progression to a more severe disease state, are associated with acquired resistance to study treatment, are associated with susceptibility to developing adverse events, or can increase the knowledge and understanding of disease biology.

Biomarkers include, but are not limited to, PD-L1 and TIGIT expression on tumor tissues and germline and somatic mutations from tumor tissue and/or from circulating tumor DNA in blood (including, but not limited to, mutation load, MSI, and MMR defects), identified through WGS and/or NGS, and plasma derived cytokines.

To assess the effect of the PD-L1/PD-1 pathway on ORR, PFS, DOR, and/or OS in the primary patient population, the relationship between protein, RNA, DNA, tumor mutational burden, and other exploratory biomarkers in tumor tissue and/or blood to efficacy, safety, PK, immunogenicity, and patient-reported outcomes (PROs) may be evaluated. Additionally, to assess the effect of the TIGIT pathway on ORR, PFS, DOR, and/or OS following in the primary population, ORR, DOR, PFS, and OS may be evaluated in a patient population whose tumors have TIGIT expression, as defined by protein and/or RNA expression.

Exploratory biomarker analyses may be performed in an effort to understand the association of these markers (e.g., TIGIT IHC status) with study treatment efficacy. The efficacy outcomes may be explored in a population of patients whose tumors have high TIGIT expression, as determined by IHC and/or RNA analysis. Exploratory analysis of WGS data may be conducted in the context of this study and explored in aggregate with data from other studies to increase researcher's understanding of disease pathobiology and guide the development of new therapeutic approaches.

Immunogenicity Analyses

To evaluate the immune response to the anti-TIGIT antagonist antibody (e.g., an anti-TIGIT antibody disclosed herein, e.g., tiragolumab) and atezolizumab, the incidence of treatment-emergent anti-drug antibodies (ADAs) and their potential impact on safety, efficacy, and pharmacokinetics (PK) will be assessed (with assessments grouped according to treatment received).

Pharmacokinetic Analyses

To characterize the pharmacokinetics of the anti-TIGIT antagonist antibody (e.g., an anti-TIGIT antibody disclosed herein, e.g., tiragolumab) when given in combination with atezolizumab, serum concentrations of the anti-TIGIT antagonist antibody are determined from subjects at different time points. Further, to characterize the pharmacokinetics of atezolizumab when atezolizumab is administered in combination with the anti-TIGIT antagonist antibody (e.g., tiragolumab) or in combination with the placebo, plasma concentration of atezolizumab is obtained from subjects at different time points during the study. PK analyses are reported and summarized using descriptive statistics.

### IX. Other Embodiments

Some embodiments of the technology described herein can be defined according to any of the following numbered embodiments:

1. A method for treating a subject having a lung cancer, the method comprising administering to the subject one or more dosing cycles of an anti-TIGIT antagonist antibody at a fixed dose of between about 30 mg to about 1200 mg every three weeks and an anti-PD-L1 antagonist antibody at a fixed dose of between about 80 mg to about 1600 mg every three weeks.

2. The method of embodiment 1, wherein the method comprises administering to the subject an anti-TIGIT antagonist antibody at a fixed dose of between about 30 mg to about 600 mg every three weeks.

3. The method of embodiment 1 or 2, wherein the method comprises administering to the subject an anti-TIGIT antagonist antibody at a fixed dose of about 600 mg every three weeks.

4. The method of any one of embodiments 1-3, wherein the anti-TIGIT antagonist antibody comprises the following hypervariable regions (HVRs):

an HVR-H1 sequence comprising the amino acid sequence of SNSAAWN (SEQ ID NO: 1);

an HVR-H2 sequence comprising the amino acid sequence of KTYYRFKWYSDYAVSVKG (SEQ ID NO: 2);

an HVR-H3 sequence comprising the amino acid sequence of ESTTYDLLAGPFDY (SEQ ID NO: 3);

an HVR-L1 sequence comprising the amino acid sequence of KSSQTVLYSSNNKKYLA (SEQ ID NO: 4);

an HVR-L2 sequence comprising the amino acid sequence of WASTRES (SEQ ID NO: 5); and

an HVR-L3 sequence comprising the amino acid sequence of QQYYSTPFT (SEQ ID NO: 6).

5. The method of any one of embodiments 1-4, wherein the anti-TIGIT antagonist antibody comprises the following light chain variable region framework regions (FRs):

an FR-L1 comprising the amino acid sequence of DIVMTQSPDSLAVSLGERATINC (SEQ ID NO: 7);

an FR-L2 comprising the amino acid sequence of WYQQKPGQPPNLLIY (SEQ ID NO: 8);

an FR-L3 comprising the amino acid sequence of GVPDRFSGSGSGTDFTLTISSLQAEDVAVYYC (SEQ ID NO: 9); and

an FR-L4 comprising the amino acid sequence of FGPGTKVEIK (SEQ ID NO: 10).

6. The method of any one of embodiments 1-5, wherein the anti-TIGIT antagonist antibody comprises the following heavy chain variable region FRs:

an FR-H1 comprising the amino acid sequence of X₁VQLQQSGPGLVKPSQTLSLTCAISGDSVS (SEQ ID NO: 11), wherein X₁ is Q or E;

an FR-H2 comprising the amino acid sequence of WIRQSPSRGLEWLG (SEQ ID NO: 12);

an FR-H3 comprising the amino acid sequence of RITINPDTSKNQFSLQLNSVTPEDTAVFYCTR (SEQ ID NO: 13); and

an FR-H4 comprising the amino acid sequence of WGQGTLVTVSS (SEQ ID NO: 14).

7. The method of embodiment 6, wherein X₁ is Q.

8. The method of embodiment 6, wherein X₁ is E.

9. The method of any one of embodiments 1-8, wherein the anti-TIGIT antagonist antibody comprises:

(a) a heavy chain variable (VH) domain comprising an amino acid sequence having at least 95% sequence identity to the amino acid sequence of SEQ ID NO: 17 or 18;

(b) a light chain variable (VL) domain comprising an amino acid sequence having at least 95% sequence identity to the amino acid sequence of SEQ ID NO: 19; or

(c) a VH domain as in (a) and a VL domain as in (b).

10. The method of any one of embodiments 1-9, wherein the anti-TIGIT antagonist antibody comprises:

a VH domain comprising the amino acid sequence of SEQ ID NO: 17 or 18; and

a VL domain comprising the amino acid sequence of SEQ ID NO: 19.

11. The method of any one of embodiments 1-10, wherein the anti-TIGIT antagonist antibody is a monoclonal antibody.

12. The method of any one of embodiments 1-11, wherein the anti-TIGIT antagonist antibody is a human antibody.

13. The method of any one of embodiments 1-12, wherein the anti-TIGIT antagonist antibody is a full-length antibody.

14. The method of any one of embodiments 1-6 and 8-13, wherein the anti-TIGIT antagonist antibody is tiragolumab.

15. The method of any one of embodiments 1-12, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

16. The method of any one of embodiments 1-15, wherein the anti-TIGIT antagonist antibody is an IgG class antibody.

17. The method of any one of embodiments 1-16, wherein the anti-TIGIT antagonist antibody is an IgG1 subclass antibody.

18. The method of any one of embodiments 1-17, the method comprises administering to the subject an anti-PD-L1 antibody at a fixed dose of about 1200 mg every three weeks.

19. The method of any one of embodiments 1-18, wherein the anti-PD-L1 antagonist antibody is atezolizumab (MPDL3280A), YW243.55.S70, MSB0010718C, MDX-1105, or MEDI4736.

20. The method of any one of embodiments 1-19, wherein the anti-PD-L1 antagonist antibody is atezolizumab.

21. The method of any one of embodiments 1-20, wherein the anti-PD-L1 antagonist antibody comprises the following HVRs:

an HVR-H1 sequence comprising the amino acid sequence of GFTFSDSWIH (SEQ ID NO: 20);

an HVR-H2 sequence comprising the amino acid sequence of AWISPYGGSTYYADSVKG (SEQ ID NO: 21);

an HVR-H3 sequence comprising the amino acid sequence of RHWPGGFDY (SEQ ID NO: 22);

an HVR-L1 sequence comprising the amino acid sequence of RASQDVSTAVA (SEQ ID NO: 23);

an HVR-L2 sequence comprising the amino acid sequence of SASFLYS (SEQ ID NO: 24); and

an HVR-L3 sequence comprising the amino acid sequence of QQYLYHPAT (SEQ ID NO: 25).

22. The method of any one of embodiments 1-21, wherein the anti-PD-L1 antagonist antibody comprises:

(a) a heavy chain variable (VH) domain comprising an amino acid sequence having at least 95% sequence identity to the amino acid sequence of SEQ ID NO: 26;

(b) a light chain variable (VL) domain comprising an amino acid sequence having at least 95% sequence identity to the amino acid sequence of SEQ ID NO: 27; or

(c) a VH domain as in (a) and a VL domain as in (b).

23. The method of any one of embodiments 1-22, wherein the anti-PD-L1 antagonist antibody comprises:

a VH domain comprising the amino acid sequence of SEQ ID NO: 26; and

a VL domain comprising the amino acid sequence of SEQ ID NO: 27.

24. The method of any one of embodiments 1-23, wherein the anti-PD-L1 antagonist antibody is a monoclonal antibody.

25. The method of any one of embodiments 1-24, wherein the anti-PD-L1 antagonist antibody is a humanized antibody.

26. The method of any one of embodiments 1-25, wherein the anti-PD-L1 antagonist antibody is a full-length antibody.

27. The method of any one of embodiments 1-25, wherein the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

28. The method of any one of embodiments 1-27, wherein the anti-PD-L1 antagonist antibody is an IgG class antibody.

29. The method of any one of embodiments 1-28, wherein the anti-PD-L1 antagonist antibody is an IgG1 subclass antibody.

30. The method of any one of embodiments 1-29, wherein the method comprises administering to the subject the anti-TIGIT antagonist antibody at a fixed dose of about 600 mg every three weeks and the anti-PD-L1 antagonist antibody at a fixed dose of about 1200 mg every three weeks.

31. The method of any one of embodiments 1-30, wherein the length of each of the one or more dosing cycles is 21 days.

32. The method of any one of embodiments 1-31, wherein the method comprises administering to the subject the anti-TIGIT antagonist antibody and the anti-PD-L1 antagonist antibody on about Day 1 of each of the one or more dosing cycles.

33. The method of any one of embodiments 1-32, wherein the method comprises administering to the subject the anti-TIGIT antagonist antibody before the anti-PD-L1 antagonist antibody.

34. The method of any one of embodiments 1-33, wherein the method comprises a first observation period following administration of the anti-TIGIT antagonist antibody and second observation period following administration of the anti-PD-L1 antagonist antibody.

35. The method of embodiment 34, wherein the first observation period and the second observation period are each between about 30 minutes to about 60 minutes in length.

36. The method of any one of embodiments 1-32, wherein the method comprises administering to the subject the anti-PD-L1 antagonist antibody before the anti-TIGIT antagonist antibody.

37. The method of any one of embodiments 1-32 and 36, wherein the method comprises a first observation period following administration of the anti-PD-L1 antagonist antibody and second observation period following administration of the anti-TIGIT antagonist antibody.

38. The method of embodiment 37, wherein the first observation period and the second observation period are each between about 30 minutes to about 60 minutes in length.

39. The method of any one of embodiments 1-32, wherein the method comprises administering to the subject the anti-TIGIT antagonist antibody and the anti-PD-L1 antagonist antibody simultaneously.

40. The method of any one of embodiments 1-39, wherein the method comprises administering to the subject the anti-TIGIT antagonist antibody and anti-PD-L1 antagonist antibody intravenously.

41. The method of any one of embodiments 1-40, wherein the method comprises administering to the subject the anti-TIGIT antagonist antibody by intravenous infusion over 60±10 minutes.

42. The method of any one of embodiments 1-41, wherein the method comprises administering to the subject the anti-PD-L1 antagonist antibody by intravenous infusion over 60±15 minutes.

43. The method of any one of embodiments 1-42, wherein a tumor sample obtained from the subject has been determined to have a detectable expression level of PD-L1.

44. The method of embodiment 43, wherein the detectable expression level of PD-L1 is a detectable protein expression level of PD-L1.

45. The method of embodiment 44, wherein the detectable protein expression level of PD-L1 has been determined by an immunohistochemical (IHC) assay.

46. The method of embodiment 45, wherein the IHC assay uses anti-PD-L1 antibody 22C3, SP142, SP263, or 28-8.

47. The method of embodiment 45 or 46, wherein the IHC assay uses anti-PD-L1 antibody 22C3.

48. The method of any one of embodiments 43-47, wherein the tumor sample has been determined to have a tumor proportion score (TPS) of greater than, or equal to, 1%.

49. The method of embodiment 48, wherein the TPS is greater than, or equal to, 1% and less than 50%.

50. The method of embodiment 48, wherein the TPS is greater than, or equal to, 50%.

51. The method of embodiment 45 or46, wherein the IHC assay uses anti-PD-L1 antibody SP142.

52. The method of any one of embodiments 43-46 and 51, wherein the tumor sample has been determined to have a detectable expression level of PD-L1 in greater than, or equal to, 1% of the tumor cells in the tumor sample.

53. The method of any one of embodiments 43-46, 51, and 52, wherein the tumor sample has been determined to have a detectable expression level of PD-L1 in greater than, or equal to, 1% and less than 5% of the tumor cells in the tumor sample.

54. The method of any one of embodiments 43-46, 51, and 52, wherein the tumor sample has been determined to have a detectable expression level of PD-L1 in greater than, or equal to, 5% and less than 50% of the tumor cells in the tumor sample.

55. The method of any one of embodiments 43-46, 51, and 52, wherein the tumor sample has been determined to have a detectable expression level of PD-L1 in greater than, or equal to, 50% of the tumor cells in the tumor sample.

56. The method of any one of embodiments 43-46 and 51-55, wherein the tumor sample has been determined to have a detectable expression level of PD-L1 in tumor-infiltrating immune cells that comprise greater than, or equal to, 1% of the tumor sample.

57. The method of any one of embodiments 43-46 and 51-56, wherein the tumor sample has been determined to have a detectable expression level of PD-L1 in tumor-infiltrating immune cells that comprise greater than, or equal to, 1% and less than 5% of the tumor sample.

58. The method of any one of embodiments 43-46 and 51-56, wherein the tumor sample has been determined to have a detectable expression level of PD-L1 in tumor-infiltrating immune cells that comprise greater than, or equal to, 5% and less than 10% of the tumor sample.

59. The method of any one of embodiments 43-46 and 51-56, wherein the tumor sample has been determined to have a detectable expression level of PD-L1 in tumor-infiltrating immune cells that comprise greater than, or equal to, 10% of the tumor sample.

60. The method of embodiment 43, wherein the detectable expression level of PD-L1 is a detectable nucleic acid expression level of PD-L1.

61. The method of embodiment 60, wherein the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

62. The method of any one of embodiments 1-61, wherein the lung cancer is a non-small cell lung cancer (NSCLC).

63. The method of any one of embodiments 1-62, wherein the lung cancer is a squamous NSCLC.

64. The method of any one of embodiments 1-62, wherein the lung cancer is a non-squamous NSCLC.

65. The method of any one of embodiments 1-64, wherein the lung cancer is a locally advanced unresectable NSCLC.

66. The method of any one of embodiments 1-65, wherein the lung cancer is a Stage IIIB NSCLC.

67. The method of any one of embodiments 1-64, wherein the lung cancer is a recurrent or metastatic NSCLC.

68. The method of any one of embodiments 1-64 and 67, wherein the lung cancer is a Stage IV NSCLC.

69. The method of any one of embodiments 1-68, wherein the subject has not been previously treated for Stage IV NSCLC.

70. The method of any one of embodiments 1-69, wherein the subject does not have a sensitizing epidermal growth factor receptor (EGFR) gene mutation or anaplastic lymphoma kinase (ALK) gene rearrangement.

71. The method of any one of embodiments 1-70, wherein the subject does not have a pulmonary lymphoepithelioma-like carcinoma subtype of NSCLC.

72. The method of any one of embodiments 1-71, wherein the subject does not have an active Epstein-Barr virus (EBV) infection or a known or suspected chronic active EBV infection.

73. The method of any one of embodiments 1-72, wherein the subject is negative for EBV IgM or negative by EBV PCR.

74. The method of any one of embodiments 1-73, wherein the subject is negative for EBV IgM and negative by EBV PCR.

75. The method of any one of embodiments 1-74, wherein the subject is positive for EBV IgG or positive for Epstein-Barr nuclear antigen (EBNA).

76. The method of any one of embodiments 1-75, wherein the subject is positive for EBV IgG and positive for EBNA.

77. The method of any one of embodiments 1-74, wherein the subject is negative for EBV IgG or negative for EBNA.

78. The method of any one of embodiments 1-74 and 77, wherein the subject is negative for EBV IgG and negative for EBNA.

79. The method of any one of embodiments 1-78, wherein the treating results in a clinical response.

80. The method of embodiment 79, wherein the clinical response is an increase in the objective response rate (ORR) of the subject as compared to a reference ORR.

81. The method of embodiment 80, wherein the reference ORR is the median ORR of a population of subjects who have received a treatment comprising an anti-PD-L1 antagonist antibody without an anti-TIGIT antagonist antibody.

82. The method of any one of embodiments 79-81, wherein the clinical response is an increase in the progression-free survival (PFS) of the subject as compared to a reference PFS time.

83. The method of any one of embodiments 79-82, wherein the reference PFS time is the median PFS time of a population of subjects who have received a treatment comprising an anti-PD-L1 antagonist antibody without an anti-TIGIT antagonist antibody.

84. A method for treating a subject having a NSCLC, the method comprising administering to the subject one or more dosing cycles of an anti-TIGIT antagonist antibody at a fixed dose of 600 mg every three weeks and atezolizumab at a fixed dose of 1200 mg every three weeks, wherein the anti-TIGIT antagonist antibody comprises:

a VH domain comprising the amino acid sequence of SEQ ID NO: 17 or 18; and

a VL domain comprising the amino acid sequence of SEQ ID NO: 19.

85. A method of treating a subject having a NSCLC, the method comprising:

(a) obtaining a tumor sample from the subject;

(b) detecting the protein expression level of PD-L1 in the tumor sample by an IHC assay using anti-PD-L1 antibody 22C3 and determining a TPS therefrom;

(c) identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1% and less than 50%, wherein the anti-TIGIT antagonist antibody comprises:

a VH domain comprising the amino acid sequence of SEQ ID NO: 17 or 18; and

a VL domain comprising the amino acid sequence of SEQ ID NO: 19; and

(d) administering to the identified subject the therapy.

86. A method of treating a subject having a NSCLC, the method comprising:

(a) obtaining a tumor sample from the subject;

(b) detecting the protein expression level of PD-L1 in the tumor sample by an IHC assay using anti-PD-L1 antibody 22C3 and determining a TPS therefrom;

(c) identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 50%, wherein the anti-TIGIT antagonist antibody comprises:

a VH domain comprising the amino acid sequence of SEQ ID NO: 17 or 18; and

a VL domain comprising the amino acid sequence of SEQ ID NO: 19; and

(d) administering to the identified subject the therapy.

87. A method of selecting a therapy for a subject having a NSCLC, the method comprising:

(a) determining a TPS from a tumor sample from the subject by an IHC assay using anti-PD-L1 antibody 22C3; and

(b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1% and less than 50%, wherein the anti-TIGIT antagonist antibody comprises:

a VH domain comprising the amino acid sequence of SEQ ID NO: 17 or 18; and

a VL domain comprising the amino acid sequence of SEQ ID NO: 19.

88. A method of selecting a therapy for a subject having a NSCLC, the method comprising:

(a) determining a TPS from a tumor sample from the subject by an IHC assay using anti-PD-L1 antibody 22C3; and

(b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 50%, wherein the anti-TIGIT antagonist antibody comprises:

a VH domain comprising the amino acid sequence of SEQ ID NO: 17 or 18; and

a VL domain comprising the amino acid sequence of SEQ ID NO: 19.

89. A method of selecting a therapy for a subject having a NSCLC, the method comprising:

(a) detecting the mutational status of the epidermal growth factor receptor (EGFR) gene and anaplastic lymphoma kinase (ALK) gene from a sample from the subject and detecting the absence of a sensitizing EGFR gene mutation or ALK gene rearrangement; and

(b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks, based on the subject not having a sensitizing EGFR gene mutation or ALK gene rearrangement, wherein the anti-TIGIT antagonist antibody comprises:

a VH domain comprising the amino acid sequence of SEQ ID NO: 17 or 18; and

a VL domain comprising the amino acid sequence of SEQ ID NO: 19.

90. A method of selecting a therapy for a subject having a NSCLC, the method comprising:

(a) biopsying a tumor sample from the subject and detecting a subtype of the NSCLC other than a pulmonary lymphoepithelioma-like carcinoma; and

(b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks, based on the subject not having a pulmonary lymphoepithelioma-like carcinoma subtype of NSCLC, wherein the anti-TIGIT antagonist antibody comprises:

a VH domain comprising the amino acid sequence of SEQ ID NO: 17 or 18; and

a VL domain comprising the amino acid sequence of SEQ ID NO: 19.

91. A method of selecting a therapy for a subject having a NSCLC, the method comprising:

(a) detecting the presence of one or more of Epstein-Barr virus (EBV) IgM, EBV IgG, Epstein-Barr nuclear antigen (EBNA), and Epstein-Barr viral particles in a sample from the subject, and

(b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks, on based on the subject being:

(i) negative for EBV IgG and/or EBNA; or

(ii) positive for EBV IgG and/or EBNA, and negative for both EBV IgM and Epstein-Barr viral particles, wherein the anti-TIGIT antagonist antibody comprises:

a VH domain comprising the amino acid sequence of SEQ ID NO: 17 or 18; and

a VL domain comprising the amino acid sequence of SEQ ID NO: 19.

92. A method for treating a subject having a NSCLC, the method comprising administering to the subject one or more dosing cycles of tiragolumab at a fixed dose of 600 mg every three weeks and atezolizumab at a fixed dose of 1200 mg every three weeks.

93. A method of treating a subject having a NSCLC, the method comprising:

(a) obtaining a tumor sample from the subject;

(b) detecting the protein expression level of PD-L1 in the tumor sample by an IHC assay using anti-PD-L1 antibody 22C3 and determining a TPS therefrom;

(c) identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1% and less than 50%; and

(d) administering to the identified subject the therapy.

94. A method of treating a subject having a NSCLC, the method comprising:

(a) obtaining a tumor sample from the subject;

(b) detecting the protein expression level of PD-L1 in the tumor sample by an IHC assay using anti-PD-L1 antibody 22C3 and determining a TPS therefrom;

(c) identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 50%; and

(d) administering to the identified subject the therapy.

95. A method of selecting a therapy for a subject having a NSCLC, the method comprising:

(a) determining a TPS from a tumor sample from the subject by an IHC assay using anti-PD-L1 antibody 22C3; and

(b) selecting for the subject a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1% and less than 50%.

96. A method of selecting a therapy for a subject having a NSCLC, the method comprising:

(a) determining a TPS from a tumor sample from the subject by an IHC assay using anti-PD-L1 antibody 22C3; and

(b) selecting for the subject a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 50%.

97. A method of selecting a therapy for a subject having a NSCLC, the method comprising:

(a) detecting the mutational status of the epidermal growth factor receptor (EGFR) gene and anaplastic lymphoma kinase (ALK) gene from a sample from the subject and detecting the absence of a sensitizing EGFR gene mutation or ALK gene rearrangement; and

(b) selecting for the subject a therapy comprising one or more dosing cycles of tiragolumab administered at a fixed dose of 600 mg every three weeks and atezolizumab administered at a fixed dose of 1200 mg every three weeks, based on the subject not having a sensitizing EGFR gene mutation or ALK gene rearrangement.

98. A method of selecting a therapy for a subject having a NSCLC, the method comprising:

(a) biopsying a tumor sample from the subject and detecting a subtype of the NSCLC other than a pulmonary lymphoepithelioma-like carcinoma; and

(b) selecting for the subject a therapy comprising one or more dosing cycles of tiragolumab administered at a fixed dose of 600 mg every three weeks and atezolizumab administered at a fixed dose of 1200 mg every three weeks, based on the subject not having a pulmonary lymphoepithelioma-like carcinoma subtype of NSCLC.

99. A method of selecting a therapy for a subject having a NSCLC, the method comprising:

(a) detecting the presence of one or more of Epstein-Barr virus (EBV) IgM, EBV IgG, Epstein-Barr nuclear antigen (EBNA), and Epstein-Barr viral particles in a sample from the subject, and

(b) selecting for the subject a therapy comprising one or more dosing cycles of tiragolumab administered at a fixed dose of 600 mg every three weeks and atezolizumab administered at a fixed dose of 1200 mg every three weeks, on based on the subject being:
(i) negative for EBV IgG and/or EBNA; or
(ii) positive for EBV IgG and/or EBNA, and negative for both EBV IgM and Epstein-Barr viral particles.

100. An anti-TIGIT antagonist antibody and an anti-PD-L1 antagonist antibody for use in a method of treating a subject having a lung cancer, wherein the method comprises administering to the subject one or more dosing cycles of the anti-TIGIT antagonist antibody at a fixed dose of between about 30 mg to about 1200 mg every three weeks and the anti-PD-L1 antagonist antibody at a fixed dose of between about 80 mg to about 1600 mg every three weeks.

101. The anti-TIGIT antagonist antibody and anti-PD-L1 antagonist antibody for use of embodiment 100, wherein the anti-TIGIT antagonist antibody is to be administered to the subject at a fixed dose of between about 30 mg to about 600 mg every three weeks.

102. The anti-TIGIT antagonist antibody and anti-PD-L1 antagonist antibody for use of embodiment 100 or 101, wherein the anti-TIGIT antagonist antibody is to be administered to the subject at a fixed dose of about 600 mg every three weeks.

103. The anti-TIGIT antagonist antibody and anti-PD-L1 antagonist antibody for use of any one of embodiments 100-102, wherein the anti-TIGIT antagonist antibody comprises the following HVRs:

an HVR-H1 sequence comprising the amino acid sequence of SNSAAWN (SEQ ID NO: 1);

an HVR-H2 sequence comprising the amino acid sequence of KTYYRFKWYSDYAVSVKG (SEQ ID NO: 2);

an HVR-H3 sequence comprising the amino acid sequence of ESTTYDLLAGPFDY (SEQ ID NO: 3);

an HVR-L1 sequence comprising the amino acid sequence of KSSQTVLYSSNNKKYLA (SEQ ID NO: 4);

an HVR-L2 sequence comprising the amino acid sequence of WASTRES (SEQ ID NO: 5); and

an HVR-L3 sequence comprising the amino acid sequence of QQYYSTPFT (SEQ ID NO: 6).

104. The anti-TIGIT antagonist antibody and anti-PD-L1 antagonist antibody for use of any one of embodiments 100-103, wherein the anti-TIGIT antagonist antibody comprises the following light chain variable region FRs:

an FR-L1 comprising the amino acid sequence of DIVMTQSPDSLAVSLGERATINC (SEQ ID NO: 7);

an FR-L2 comprising the amino acid sequence of WYQQKPGQPPNLLIY (SEQ ID NO: 8);

an FR-L3 comprising the amino acid sequence of GVPDRFSGSGSGTDFTLTISSLQAEDVAVYYC (SEQ ID NO: 9); and

an FR-L4 comprising the amino acid sequence of FGPGTKVEIK (SEQ ID NO: 10).

105. The anti-TIGIT antagonist antibody and anti-PD-L1 antagonist antibody for use of any one of embodiments 100-104, wherein the anti-TIGIT antagonist antibody comprises the following heavy chain variable region FRs:

an FR-H1 comprising the amino acid sequence of X₁VQLQQSGPGLVKPSQTLSLTCAISGDSVS (SEQ ID NO: 11), wherein X₁ is Q or E;

an FR-H2 comprising the amino acid sequence of WIRQSPSRGLEWLG (SEQ ID NO: 12);

an FR-H3 comprising the amino acid sequence of RITINPDTSKNQFSLQLNSVTPEDTAVFYCTR (SEQ ID NO: 13); and

an FR-H4 comprising the amino acid sequence of WGQGTLVTVSS (SEQ ID NO: 14).

106. The anti-TIGIT antagonist antibody and anti-PD-L1 antagonist antibody for use of embodiment 105, wherein X₁ is Q.

107. The anti-TIGIT antagonist antibody and anti-PD-L1 antagonist antibody of embodiment 105, wherein X₁ is E.

108. The anti-TIGIT antagonist antibody and anti-PD-L1 antagonist antibody for use of any one of embodiments 100-107, wherein the anti-TIGIT antagonist antibody comprises:

(a) a heavy chain variable (VH) domain comprising an amino acid sequence having at least 95% sequence identity to the amino acid sequence of SEQ ID NO: 17 or 18;

(b) a light chain variable (VL) domain comprising an amino acid sequence having at least 95% sequence identity to the amino acid sequence of SEQ ID NO: 19; or

(c) a VH domain as in (a) and a VL domain as in (b).

109. The anti-TIGIT antagonist antibody and anti-PD-L1 antagonist antibody for use of any one of embodiments 100-108, wherein the anti-TIGIT antagonist antibody comprises:

a VH domain comprising the amino acid sequence of SEQ ID NO: 17 or 18; and

a VL domain comprising the amino acid sequence of SEQ ID NO: 19.

110. The anti-TIGIT antagonist antibody and anti-PD-L1 antagonist antibody for use of any one of embodiments 100-109, wherein the anti-TIGIT antagonist antibody is a monoclonal antibody.

111. The anti-TIGIT antagonist antibody and anti-PD-L1 antagonist antibody for use of any one of embodiments 100-110, wherein the anti-TIGIT antagonist antibody is a human antibody.

112. The anti-TIGIT antagonist antibody and anti-PD-L1 antagonist antibody for use of any one of embodiments 100-111, wherein the anti-TIGIT antagonist antibody is a full-length antibody.

113. The anti-TIGIT antagonist antibody and anti-PD-L1 antagonist antibody for use of any one of embodiments 100-105 and 107-112 wherein the anti-TIGIT antagonist antibody is tiragolumab.

114. The anti-TIGIT antagonist antibody and anti-PD-L1 antagonist antibody for use of any one of embodiments 100-111, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

115. The anti-TIGIT antagonist antibody and anti-PD-L1 antagonist antibody for use of any one of embodiments 100-114, wherein the anti-TIGIT antagonist antibody is an IgG class antibody.

116. The anti-TIGIT antagonist antibody and anti-PD-L1 antagonist antibody for use of any one of embodiments 100-115, wherein the anti-TIGIT antagonist antibody is an IgG1 subclass antibody.

117. The anti-TIGIT antagonist antibody and anti-PD-L1 antagonist antibody for use of any one of embodiments 100-116, wherein the anti-PD-L1 antagonist antibody is to be administered to the subject at a fixed dose of about 1200 mg every three weeks.

118. The anti-TIGIT antagonist antibody and anti-PD-L1 antagonist antibody for use of any one of embodiments 100-117, wherein the anti-PD-L1 antagonist antibody is atezolizumab (MPDL3280A), YW243.55.S70, MSB0010718C, MDX-1105, or MEDI4736.

119. The anti-TIGIT antagonist antibody and anti-PD-L1 antagonist antibody for use of any one of embodiments 100-118, wherein the anti-PD-L1 antagonist antibody is atezolizumab.

120. The anti-TIGIT antagonist antibody and anti-PD-L1 antagonist antibody for use of any one of embodiments 100-119, wherein the anti-PD-L1 antagonist antibody comprises the following HVRs:

an HVR-H1 sequence comprising the amino acid sequence of GFTFSDSWIH (SEQ ID NO: 20);

an HVR-H2 sequence comprising the amino acid sequence of AWISPYGGSTYYADSVKG (SEQ ID NO: 21);

an HVR-H3 sequence comprising the amino acid sequence of RHWPGGFDY (SEQ ID NO: 22);

an HVR-L1 sequence comprising the amino acid sequence of RASQDVSTAVA (SEQ ID NO: 23);

an HVR-L2 sequence comprising the amino acid sequence of SASFLYS (SEQ ID NO: 24); and

an HVR-L3 sequence comprising the amino acid sequence of QQYLYHPAT (SEQ ID NO: 25).

121. The anti-TIGIT antagonist antibody and anti-PD-L1 antagonist antibody for use of any one of embodiments 100-120, wherein the anti-PD-L1 antagonist antibody comprises:

(a) a heavy chain variable (VH) domain comprising an amino acid sequence having at least 95% sequence identity to the amino acid sequence of SEQ ID NO: 26;

(b) a light chain variable (VL) domain comprising an amino acid sequence having at least 95% sequence identity to the amino acid sequence of SEQ ID NO: 27; or

(c) a VH domain as in (a) and a VL domain as in (b).

122. The anti-TIGIT antagonist antibody and anti-PD-L1 antagonist antibody for use of any one of embodiments 100-121, wherein the anti-PD-L1 antagonist antibody comprises:

a VH domain comprising the amino acid sequence of SEQ ID NO: 26; and

a VL domain comprising the amino acid sequence of SEQ ID NO: 27.

123. The anti-TIGIT antagonist antibody and anti-PD-L1 antagonist antibody for use of any one of embodiments 100-122, wherein the anti-PD-L1 antagonist antibody is a monoclonal antibody.

124. The anti-TIGIT antagonist antibody and anti-PD-L1 antagonist antibody for use of any one of embodiments 100-123, wherein the anti-PD-L1 antagonist antibody is a humanized antibody.

125. The anti-TIGIT antagonist antibody and anti-PD-L1 antagonist antibody for use of any one of embodiments 100-124, wherein the anti-PD-L1 antagonist antibody is a full-length antibody.

126. The anti-TIGIT antagonist antibody and anti-PD-L1 antagonist antibody for use of any one of embodiments 100-124, wherein the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

127. The anti-TIGIT antagonist antibody and anti-PD-L1 antagonist antibody for use of any one of embodiments 100-126, wherein the anti-PD-L1 antagonist antibody is an IgG class antibody.

128. The anti-TIGIT antagonist antibody and anti-PD-L1 antagonist antibody for use of any one of embodiments 100-127, wherein the anti-PD-L1 antagonist antibody is an IgG1 subclass antibody.

129. The anti-TIGIT antagonist antibody and anti-PD-L1 antagonist antibody for use of any one of embodiments 100-128, wherein the anti-TIGIT antagonist antibody is to be administered to the subject at a fixed dose of about 600 mg every three weeks and the anti-PD-L1 antagonist antibody is to be administered to the subject at a fixed dose of about 1200 mg every three weeks.

130. The anti-TIGIT antagonist antibody and anti-PD-L1 antagonist antibody for use of any one of embodiments 100-129, wherein the length of each of the one or more dosing cycles is 21 days.

131. The anti-TIGIT antagonist antibody and anti-PD-L1 antibody for use of any one of embodiments 100-130, wherein the anti-TIGIT antagonist antibody and anti-PD-L1 antagonist antibody are to be administered to the subject on about Day 1 of each of the one or more dosing cycles.

132. The anti-TIGIT antagonist antibody and anti-PD-L1 antagonist antibody for use of any one of embodiments 100-131, wherein the anti-TIGIT antagonist antibody is to be administered to the subject before the anti-PD-L1 antagonist antibody.

133. The anti-TIGIT antagonist antibody and anti-PD-L1 antagonist antibody for use of any one of embodiments 100-132, wherein a first observation period is to follow administration of the anti-TIGIT antagonist antibody and second observation period is to follow administration of the anti-PD-L1 antagonist antibody.

134. The anti-TIGIT antagonist antibody and anti-PD-L1 antagonist antibody for use of embodiment 133, wherein the first observation period and the second observation period are each between about 30 minutes to about 60 minutes in length.

135. The anti-TIGIT antagonist antibody and anti-PD-L1 antagonist antibody for use of any one of embodiments 100-131, wherein the anti-PD-L1 antagonist antibody is to be administered to the subject before the anti-TIGIT antagonist antibody.

136. The anti-TIGIT antagonist antibody and anti-PD-L1 antagonist antibody for use of any one of embodiments 100-131 and 135, wherein a first observation period is to follow administration of the anti-PD-L1 antagonist antibody and second observation period is to follow administration of the anti-TIGIT antagonist antibody.

137. The anti-TIGIT antagonist antibody and anti-PD-L1 antagonist antibody for use of embodiment 136, wherein the first observation period and the second observation period are each between about 30 minutes to about 60 minutes in length.

138. The anti-TIGIT antagonist antibody and anti-PD-L1 antagonist antibody for use of any one of embodiments 100-131, wherein the anti-TIGIT antagonist antibody is to be administered to the subject simultaneously with the anti-PD-L1 antagonist antibody.

139. The anti-TIGIT antagonist antibody and anti-PD-L1 antagonist antibody for use of any one of embodiments 100-138, wherein the anti-TIGIT antagonist antibody and anti-PD-L1 antagonist antibody are to be administered to the subject intravenously.

140. The anti-TIGIT antagonist antibody and anti-PD-L1 antagonist antibody for use of any one of embodiments 100-139, wherein the anti-TIGIT antagonist antibody is to be administered to the subject by intravenous infusion over 60±10 minutes.

141. The anti-TIGIT antagonist antibody and anti-PD-L1 antagonist antibody for use of any one of embodiments 100-140, wherein the anti-PD-L1 antagonist antibody is to be administered to the subject by intravenous infusion over 60±15 minutes.

142. The anti-TIGIT antagonist antibody and anti-PD-L1 antagonist antibody for use of any one of embodiments 100-141, wherein a tumor sample obtained from the subject has been determined to have a detectable expression level of PD-L1.

143. The anti-TIGIT antagonist antibody and anti-PD-L1 antibody for use of embodiment 142, wherein the detectable expression level of PD-L1 is a detectable protein expression level of PD-L1.

144. The anti-TIGIT antagonist antibody and anti-PD-L1 antagonist antibody for use of embodiment 143, wherein the detectable protein expression level of PD-L1 has been determined by an immunohistochemical (IHC) assay.

145. The anti-TIGIT antagonist antibody and anti-PD-L1 antagonist antibody for use of embodiment 144, wherein the IHC assay uses anti-PD-L1 antibody 22C3, SP142, SP263, or 28-8.

146. The anti-TIGIT antagonist antibody and anti-PD-L1 antagonist antibody for use of embodiment 144 or 145, wherein the IHC assay uses anti-PD-L1 antibody 22C3.

147. The anti-TIGIT antagonist antibody and anti-PD-L1 antagonist antibody for use of any one of embodiments 142-146, wherein the tumor sample has been determined to have a tumor proportion score (TPS) of greater than, or equal to, 1%.

148. The anti-TIGIT antagonist antibody and anti-PD-L1 antagonist antibody for use of embodiment 147, wherein the TPS is greater than, or equal to, 1% and less than 50%.

149. The anti-TIGIT antagonist antibody and anti-PD-L1 antagonist antibody for use of embodiment 147, wherein the TPS is greater than, or equal to, 50%.

150. The anti-TIGIT antagonist antibody and anti-PD-L1 antagonist antibody for use of any one of embodiments 144 or 145, wherein the IHC assay uses anti-PD-L1 antibody SP142.

151. The anti-TIGIT antagonist antibody and anti-PD-L1 antagonist antibody for use of any one of embodiments 142-145 and 150, wherein the tumor sample has been determined to have a detectable expression level of PD-L1 in greater than, or equal to, 1% of the tumor cells in the tumor sample.

152. The anti-TIGIT antagonist antibody and anti-PD-L1 antagonist antibody for use of any one of embodiments 142-145, 150, and 151, wherein the tumor sample has been determined to have a detectable expression level of PD-L1 in greater than, or equal to, 1% and less than 5% of the tumor cells in the tumor sample.

153. The anti-TIGIT antagonist antibody and anti-PD-L1 antagonist antibody for use of any one of embodiments 142-145, 150, and 151, wherein the tumor sample has been determined to have a detectable expression level of PD-L1 in greater than, or equal to, 5% and less than 50% of the tumor cells in the tumor sample.

154. The anti-TIGIT antagonist antibody and anti-PD-L1 antagonist antibody for use of any one of embodiments 142-145, 150, and 151, wherein the tumor sample has been determined to have a detectable expression level of PD-L1 in greater than, or equal to, 50% of the tumor cells in the tumor sample.

155. The anti-TIGIT antagonist antibody and anti-PD-L1 antagonist antibody for use of any one of embodiments 142-145 and 150-154, wherein the tumor sample has been determined to have a detectable expression level of PD-L1 in tumor-infiltrating immune cells that comprise greater than, or equal to, 1% of the tumor sample.

156. The anti-TIGIT antagonist antibody and anti-PD-L1 antagonist antibody for use of any one of embodiments 142-145 and 150-155, wherein the tumor sample has been determined to have a detectable expression level of PD-L1 in tumor-infiltrating immune cells that comprise greater than, or equal to, 1% and less than 5% of the tumor sample.

157. The anti-TIGIT antagonist antibody and anti-PD-L1 antagonist antibody for use of any one of embodiments 142-145 and 150-155, wherein the tumor sample has been determined to have a detectable expression level of PD-L1 in tumor-infiltrating immune cells that comprise greater than, or equal to, 5% and less than 10% of the tumor sample.

158. The anti-TIGIT antagonist antibody and anti-PD-L1 antagonist antibody for use of any one of embodiments 142-145 and 150-155, wherein the tumor sample has been determined to have a detectable expression level of PD-L1 in tumor-infiltrating immune cells that comprise greater than, or equal to, 10% of the tumor sample.

159. The anti-TIGIT antagonist antibody and anti-PD-L1 antagonist antibody for use of embodiment 142, wherein the detectable expression level of PD-L1 is a detectable nucleic acid expression level of PD-L1.

160. The anti-TIGIT antagonist antibody and anti-PD-L1 antagonist antibody for use of embodiment 159, wherein the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

161. The anti-TIGIT antagonist antibody and anti-PD-L1 antagonist antibody for use of any one of embodiments 100-160, wherein the lung cancer is a non-small cell lung cancer (NSCLC).

162. The anti-TIGIT antagonist antibody and anti-PD-L1 antagonist antibody for use of any one of embodiments 100-161, wherein the lung cancer is a squamous NSCLC.

163. The anti-TIGIT antagonist antibody and anti-PD-L1 antagonist antibody for use of any one of embodiments 100-161, wherein the lung cancer is a non-squamous NSCLC.

164. The anti-TIGIT antagonist antibody and anti-PD-L1 antagonist antibody for use of any one of embodiments 100-163, wherein the lung cancer is a locally advanced unresectable NSCLC.

165. The anti-TIGIT antagonist antibody and anti-PD-L1 antagonist antibody for use of any one of embodiments 100-164, wherein the lung cancer is a Stage IIIB NSCLC.

166. The anti-TIGIT antagonist antibody and anti-PD-L1 antagonist antibody for use of any one of embodiments any one of embodiments 100-163, wherein the lung cancer is a recurrent or metastatic NSCLC.

167. The anti-TIGIT antagonist antibody and anti-PD-L1 antagonist antibody for use of any one of embodiments 100-163 and 166, wherein the lung cancer is a Stage IV NSCLC.

168. The anti-TIGIT antagonist antibody and anti-PD-L1 antagonist antibody for use of any one of embodiments 100-167, wherein the subject has not been previously treated for Stage IV NSCLC.

169. The anti-TIGIT antagonist antibody and anti-PD-L1 antagonist antibody for use of any one of embodiments 100-168, wherein the subject does not have a sensitizing epidermal growth factor receptor (EGFR) gene mutation or anaplastic lymphoma kinase (ALK) gene rearrangement.

170. The anti-TIGIT antagonist antibody and anti-PD-L1 antagonist antibody for use of any one of embodiments 100-169, wherein the subject does not have a pulmonary lymphoepithelioma-like carcinoma subtype of NSCLC.

171. The anti-TIGIT antagonist antibody and anti-PD-L1 antagonist antibody for use of any one of embodiments 100-170, wherein the subject does not have an active EBV infection or a known or suspected chronic active EBV infection.

172. The anti-TIGIT antagonist antibody and anti-PD-L1 antagonist antibody for use of any one of embodiments 100-171, wherein the subject is negative for EBV IgM or negative by EBV PCR.

173. The anti-TIGIT antagonist antibody and anti-PD-L1 antagonist antibody for use of any one of embodiments 100-172, wherein the subject is negative for EBV IgM and negative by EBV PCR.

174. The anti-TIGIT antagonist antibody and anti-PD-L1 antagonist antibody for use of any one of embodiments 100-173, wherein the subject is positive for EBV IgG or positive for EBNA.

175. The anti-TIGIT antagonist antibody and anti-PD-L1 antagonist antibody for use of any one of embodiments 100-174, wherein the subject is positive for EBV IgG and positive for EBNA.

176. The anti-TIGIT antagonist antibody and anti-PD-L1 antagonist antibody for use of any one of embodiments 100-173, wherein the subject is negative for EBV IgG or negative for EBNA.

177. The anti-TIGIT antagonist antibody and anti-PD-L1 antagonist antibody for use of any one of embodiments 100-173 and 176, wherein the subject is negative for EBV IgG and negative for EBNA.

178. The anti-TIGIT antagonist antibody and anti-PD-L1 antagonist antibody for use of any one of embodiments 100-177, wherein administration of the anti-TIGIT antagonist antibody and anti-PD-L1 antagonist antibody results in a clinical response.

179. The anti-TIGIT antagonist antibody and anti-PD-L1 antagonist antibody for use of any one of embodiments 100-178, wherein the clinical response is an increase in the objective response rate (ORR) of the subject as compared to a reference ORR.

180. The anti-TIGIT antagonist antibody and anti-PD-L1 antagonist antibody for use of any one of embodiments 100-179, wherein the reference ORR is the median ORR of a population of subjects who have received a treatment comprising an anti-PD-L1 antagonist antibody without an anti-TIGIT antagonist antibody.

181. The anti-TIGIT antagonist antibody and anti-PD-L1 antagonist antibody for use of any one of embodiments 100-180, wherein the clinical response is an increase in the progression-free survival (PFS) of the subject as compared to a reference PFS time.

182. The anti-TIGIT antagonist antibody and anti-PD-L1 antagonist antibody for use of any one of embodiments 100-181, wherein the reference PFS time is the median PFS time of a population of subjects who have received a treatment comprising an anti-PD-L1 antagonist antibody without an anti-TIGIT antagonist antibody.

183. An anti-TIGIT antagonist antibody and atezolizumab for use in a method of treating a subject having a NSCLC, wherein the method comprises administering to the subject one or more dosing cycles of an anti-TIGIT antagonist antibody at a fixed dose of 600 mg every three weeks and atezolizumab at a fixed dose of 1200 mg every three weeks, wherein the anti-TIGIT antagonist antibody comprises:

a VH domain comprising the amino acid sequence of SEQ ID NO: 17 or 18; and

a VL domain comprising the amino acid sequence of SEQ ID NO: 19.

184. Tiragolumab and atezolizumab for use in a method of treating a subject having a NSCLC, wherein the method comprises administering to the subject one or more dosing cycles of tiragolumab at a fixed dose of 600 mg every three weeks and atezolizumab at a fixed dose of 1200 mg every three weeks.

185. Use of an anti-TIGIT antagonist antibody and an anti-PD-L1 antagonist antibody in the manufacture of a medicament for use in a method of treating a subject having a lung cancer, wherein the method comprises administering to the subject one or more dosing cycles of the medicament, and wherein the medicament is formulated for administration of the anti-TIGIT antagonist antibody at a fixed dose of between about 30 mg to about 1200 mg every three weeks and the anti-PD-L1 antagonist antibody at a fixed dose of between about 80 mg to about 1600 mg every three weeks.

186. Use of an anti-TIGIT antagonist antibody in the manufacture of a medicament for use in a method of treating a subject having a lung cancer, wherein the method comprises administering to the subject one or more dosing cycles of the medicament and an anti-PD-L1 antagonist antibody, and wherein the medicament is formulated for administration of the anti-TIGIT antagonist antibody at a fixed dose of between about 30 mg to about 1200 mg every three weeks and the anti-PD-L1 antagonist antibody is to be administered at a fixed dose of between about 80 mg to about 1600 mg every three weeks.

187. Use of an anti-PD-L1 antagonist antibody in the manufacture of a medicament for use in a method of treating a subject having a lung cancer, wherein the method comprises administering to the subject one or more dosing cycles of the medicament and an anti-TIGIT antagonist antibody, and wherein the medicament is formulated for administration of the anti-PD-L1 antagonist antibody at a fixed dose of between about 80 mg to about 1600 mg every three weeks and the anti-TIGIT antagonist antibody is to be administered at a fixed dose of between about 30 mg to about 1200 mg every three weeks.

188. The use of any one of embodiments 185-187, wherein the anti-TIGIT antagonist antibody is to be administered to the subject at a fixed dose of between about 30 mg to about 600 mg every three weeks.

189. The use of any one of embodiments 185-188, wherein the anti-TIGIT antagonist antibody is to be administered to the subject at a fixed dose of about 600 mg every three weeks.

190. The use of any one of embodiments 185-189, wherein the anti-TIGIT antagonist antibody comprises the following hypervariable regions (HVRs):

an HVR-H1 sequence comprising the amino acid sequence of SNSAAWN (SEQ ID NO: 1);

an HVR-H2 sequence comprising the amino acid sequence of KTYYRFKWYSDYAVSVKG (SEQ ID NO: 2);

an HVR-H3 sequence comprising the amino acid sequence of ESTTYDLLAGPFDY (SEQ ID NO: 3);

an HVR-L1 sequence comprising the amino acid sequence of KSSQTVLYSSNNKKYLA (SEQ ID NO: 4);

an HVR-L2 sequence comprising the amino acid sequence of WASTRES (SEQ ID NO: 5); and

an HVR-L3 sequence comprising the amino acid sequence of QQYYSTPFT (SEQ ID NO: 6).

191. The use of any one of embodiments 185-190, wherein the anti-TIGIT antagonist antibody comprises the following light chain variable region framework regions (FRs):

an FR-L1 comprising the amino acid sequence of DIVMTQSPDSLAVSLGERATINC (SEQ ID NO: 7);

an FR-L2 comprising the amino acid sequence of WYQQKPGQPPNLLIY (SEQ ID NO: 8);

an FR-L3 comprising the amino acid sequence of GVPDRFSGSGSGTDFTLTISSLQAEDVAVYYC (SEQ ID NO: 9); and

an FR-L4 comprising the amino acid sequence of FGPGTKVEIK (SEQ ID NO: 10).

192. The use of any one of embodiments 185-191, wherein the anti-TIGIT antagonist antibody comprises the following heavy chain variable region FRs:

an FR-H1 comprising the amino acid sequence of X₁VQLQQSGPGLVKPSQTLSLTCAISGDSVS (SEQ ID NO: 11), wherein X₁ is Q or E;

an FR-H2 comprising the amino acid sequence of WIRQSPSRGLEWLG (SEQ ID NO: 12);

an FR-H3 comprising the amino acid sequence of RITINPDTSKNQFSLQLNSVTPEDTAVFYCTR (SEQ ID NO: 13); and

an FR-H4 comprising the amino acid sequence of WGQGTLVTVSS (SEQ ID NO: 14).

193. The use of embodiment 192, wherein X₁ is Q.

194. The use of embodiment 192, wherein X₁ is E.

195. The use of any one of embodiments 185-194, wherein the anti-TIGIT antagonist antibody comprises:

(a) a heavy chain variable (VH) domain comprising an amino acid sequence having at least 95% sequence identity to the amino acid sequence of SEQ ID NO: 17 or 18;

(b) a light chain variable (VL) domain comprising an amino acid sequence having at least 95% sequence identity to the amino acid sequence of SEQ ID NO: 19; or

(c) a VH domain as in (a) and a VL domain as in (b).

196. The use of any one of embodiments 185-195, wherein the anti-TIGIT antagonist antibody comprises:

(a) a heavy chain variable (VH) domain comprising an amino acid sequence having at least 95% sequence identity to the amino acid sequence of SEQ ID NO: 17 or 18;

(b) a light chain variable (VL) domain comprising an amino acid sequence having at least 95% sequence identity to the amino acid sequence of SEQ ID NO: 19; or

(c) a VH domain as in (a) and a VL domain as in (b).

197. The use of any one of embodiments 185-196, wherein the anti-TIGIT antagonist antibody is a monoclonal antibody.

198. The use of any one of embodiments 185-197, wherein the anti-TIGIT antagonist antibody is a human antibody.

199. The use of any one of embodiments 185-198, wherein the anti-TIGIT antagonist antibody is a full-length antibody.

200. The use of any one of embodiments 185-192 and 194-199, wherein the anti-TIGIT antagonist antibody is tiragolumab.

201. The use of any one of embodiments 185-198, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

202. The use of any one of embodiments 185-201, wherein the anti-TIGIT antagonist antibody is an IgG class antibody.

203. The use of any one of embodiments 185-202, wherein the anti-TIGIT antagonist antibody is an IgG1 subclass antibody.

204. The use of any one of embodiments 185-203, wherein the anti-PD-L1 antagonist antibody is to be administered to the subject at a fixed dose of about 1200 mg every three weeks.

205. The use of any one of embodiments 185-204, wherein the anti-PD-L1 antagonist antibody is atezolizumab (MPDL3280A), YW243.55.S70, MSB0010718C, MDX-1105, or MEDI4736.

206. The use of any one of embodiments 185-205, wherein the anti-PD-L1 antagonist antibody is atezolizumab.

207. The use of any one of embodiments 185-204, wherein the anti-PD-L1 antagonist antibody comprises the following HVRs:

an HVR-H1 sequence comprising the amino acid sequence of GFTFSDSWIH (SEQ ID NO: 20);

an HVR-H2 sequence comprising the amino acid sequence of AWISPYGGSTYYADSVKG (SEQ ID NO: 21);

an HVR-H3 sequence comprising the amino acid sequence of RHWPGGFDY (SEQ ID NO: 22);

an HVR-L1 sequence comprising the amino acid sequence of RASQDVSTAVA (SEQ ID NO: 23);

an HVR-L2 sequence comprising the amino acid sequence of SASFLYS (SEQ ID NO: 24); and

an HVR-L3 sequence comprising the amino acid sequence of QQYLYHPAT (SEQ ID NO: 25).

208. The use of any one of embodiments 185-207, wherein the anti-PD-L1 antagonist antibody comprises:

(a) a heavy chain variable (VH) domain comprising an amino acid sequence having at least 95% sequence identity to the amino acid sequence of SEQ ID NO: 26;

(b) a light chain variable (VL) domain comprising an amino acid sequence having at least 95% sequence identity to the amino acid sequence of SEQ ID NO: 27; or

(c) a VH domain as in (a) and a VL domain as in (b).

209. The use of any one of embodiments 185-208, wherein the anti-PD-L1 antagonist antibody comprises:

a VH domain comprising the amino acid sequence of SEQ ID NO: 26; and

a VL domain comprising the amino acid sequence of SEQ ID NO: 27.

210. The use of any one of embodiments 185-209, wherein the anti-PD-L1 antagonist antibody is a monoclonal antibody.

211. The use of any one of embodiments 185-210, wherein the anti-PD-L1 antagonist antibody is a humanized antibody.

212. The use of any one of embodiments 185-211, wherein the anti-PD-L1 antagonist antibody is a full-length antibody.

213. The use of any one of embodiments 185-211, wherein the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

214. The use of any one of embodiments 185-213, wherein the anti-PD-L1 antagonist antibody is an IgG class antibody.

215. The use of any one of embodiments 185-214, wherein the anti-PD-L1 antagonist antibody is an IgG1 subclass antibody.

216. The use of any one of embodiments 185-215, wherein the anti-TIGIT antagonist antibody is to be administered to the subject at a fixed dose of about 600 mg of every three weeks and the anti-PD-L1 antagonist antibody is to be administered to the subject at a fixed dose of about 1200 mg every three weeks.

217. The use of any one of embodiments 185-216, wherein the length of each of the one or more dosing cycles is 21 days.

218. The use of any one of embodiments 185-217, wherein the anti-TIGIT antagonist antibody and anti-PD-L1 antagonist antibody are to be administered to the subject on about Day 1 of each of the one or more dosing cycles.

219. The use of any one of embodiments 185-218, wherein the anti-TIGIT antagonist antibody is to be administered to the subject before the anti-PD-L1 antagonist antibody.

220. The use of any one of embodiments 185-219, wherein a first observation period is to follow administration of the anti-TIGIT antagonist antibody and second observation period is to follow administration of the anti-PD-L1 antagonist antibody.

221. The use of embodiment 220, wherein the first observation period and the second observation period are each between about 30 minutes to about 60 minutes in length.

222. The use of any one of embodiments 185-218, wherein the anti-PD-L1 antagonist antibody is to be administered to the subject before the anti-TIGIT antagonist antibody.

223. The use of any one of embodiments 185-218 and 222, wherein a first observation period is to follow administration of the anti-PD-L1 antagonist antibody and second observation period is to follow administration of the anti-TIGIT antagonist antibody.

224. The use of embodiment 223, wherein the first observation period and the second observation period are each between about 30 minutes to about 60 minutes in length.

225. The use of any one of embodiments 185-218, wherein the anti-TIGIT antagonist antibody is to be administered to the subject simultaneously with the anti-PD-L1 antagonist antibody.

226. The use of any one of embodiments 185-225, wherein the anti-TIGIT antagonist antibody and anti-PD-L1 antagonist antibody are to be administered to the subject intravenously.

227. The use of any one of embodiments 185-226, wherein the anti-TIGIT antagonist antibody is to be administered to the subject by intravenous infusion over 60±10 minutes.

228. The use of any one of embodiments 185-227, wherein the anti-PD-L1 antagonist antibody is to be administered to the subject by intravenous infusion over 60±15 minutes.

229. The use of any one of embodiments 185-228, wherein a tumor sample obtained from the subject has been determined to have a detectable expression level of PD-L1.

230. The use of embodiment 229, wherein the detectable expression level of PD-L1 is a detectable protein expression level of PD-L1.

231. The use of embodiment 230, wherein the detectable protein expression level of PD-L1 has been determined by an immunohistochemical (IHC) assay.

232. The use of embodiment 231, wherein the IHC assay uses anti-PD-L1 antibody 22C3, SP142, SP263, or 28-8.

233. The use of embodiment 231 or 232, wherein the IHC assay uses anti-PD-L1 antibody 22C3.

234. The use of any one of embodiments 230-233, wherein the tumor sample has been determined to have a tumor proportion score (TPS) of greater than, or equal to, 1%.

235. The use of embodiment 234, wherein the TPS is greater than, or equal to, 1% and less than 50%.

236. The use of embodiment 234, wherein the TPS is greater than, or equal to, 50%.

237. The use of embodiment 231 or 232, wherein the IHC assay uses anti-PD-L1 antibody SP142.

238. The use of any one of embodiments 230-232 and 237, wherein the tumor sample has been determined to have a detectable expression level of PD-L1 in greater than, or equal to, 1% of the tumor cells in the tumor sample.

239. The use of any one of embodiments 230-232, 237 and 238, wherein the tumor sample has been determined to have a detectable expression level of PD-L1 in greater than, or equal to, 1% and less than 5% of the tumor cells in the tumor sample.

240. The use of any one of embodiments 230-232, 237 and 238, wherein the tumor sample has been determined to have a detectable expression level of PD-L1 in greater than, or equal to, 5% and less than 50% of the tumor cells in the tumor sample.

241. The use of any one of embodiments 230-232, 237 and 238, wherein the tumor sample has been determined to have a detectable expression level of PD-L1 in greater than, or equal to, 50% of the tumor cells in the tumor sample.

242. The use of any one of embodiments 230-232 and 237-241, wherein the tumor sample has been determined to have a detectable expression level of PD-L1 in tumor-infiltrating immune cells that comprise greater than, or equal to, 1% of the tumor sample.

243. The use of any one of embodiments 230-232 and 237-242, wherein the tumor sample has been determined to have a detectable expression level of PD-L1 in tumor-infiltrating immune cells that comprise greater than, or equal to, 1% and less than 5% of the tumor sample.

244. The use of any one of embodiments 230-232 and 237-242, wherein the tumor sample has been determined to have a detectable expression level of PD-L1 in tumor-infiltrating immune cells that comprise greater than, or equal to, 5% and less than 10% of the tumor sample.

245. The use of any one of embodiments 230-232 and 237-242, wherein the tumor sample has been determined to have a detectable expression level of PD-L1 in tumor-infiltrating immune cells that comprise greater than, or equal to, 10% of the tumor sample.

246. The use of embodiment 231, wherein the detectable expression level of PD-L1 is a detectable nucleic acid expression level of PD-L1.

247. The use of embodiment 246, wherein the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

248. The use of any one of embodiments 185-247, wherein the lung cancer is a non-small cell lung cancer (NSCLC).

249. The use of any one of embodiments 185-248, wherein the lung cancer is a squamous NSCLC.

250. The use of any one of embodiments 185-248, wherein the lung cancer is a non-squamous NSCLC.

251. The use of any one of any one of embodiments 185-250, wherein the lung cancer is a locally advanced unresectable NSCLC.

252. The use of any one of embodiments 185-251, wherein the lung cancer is a Stage IIIB NSCLC.

253. The use of any one of embodiments 185-251, wherein the lung cancer is a recurrent or metastatic NSCLC.

254. The use of any one of embodiments 185-251 and 253, wherein the lung cancer is a Stage IV NSCLC.

255. The use of any one of embodiments 185-254, wherein the subject has not been previously treated for Stage IV NSCLC.

256. The use of any one of embodiments 185-255, wherein the subject does not have a sensitizing epidermal growth factor receptor (EGFR) gene mutation or anaplastic lymphoma kinase (ALK) gene rearrangement.

257. The use of any one of embodiments 185-256, wherein the subject does not have a pulmonary lymphoepithelioma-like carcinoma subtype of NSCLC.

258. The use of any one of embodiments 185-257, wherein the subject does not have an active EBV infection or a known or suspected chronic active EBV infection.

259. The use of any one of embodiments 185-258, wherein the subject is negative for EBV IgM or negative by EBV PCR.

260. The use of any one of embodiments 185-259, wherein the subject is negative for EBV IgM and negative by EBV PCR.

261. The use of any one of embodiments 185-260, wherein the subject is positive for EBV IgG or positive for EBNA.

262. The use of any one of embodiments 185-261, wherein the subject is positive for EBV IgG and positive for EBNA.

263. The use of any one of embodiments 185-260, wherein the subject is negative for EBV IgG or negative for EBNA.

264. The use of any one of embodiments 185-260 and 263, wherein the subject is negative for EBV IgG and negative for EBNA.

265. The use of any one of embodiments 185-264, wherein administration of the anti-TIGIT antagonist antibody and anti-PD-L1 antagonist antibody results in a clinical response.

266. The use of any one of embodiments 185-265, wherein the clinical response is an increase in the objective response rate (ORR) of the subject as compared to a reference ORR.

267. The use of any one of embodiments 185-266, wherein the reference ORR is the median ORR of a population of subjects who have received a treatment comprising an anti-PD-L1 antagonist antibody without an anti-TIGIT antagonist antibody.

268. The use of any one of embodiments 185-267, wherein the clinical response is an increase in the progression-free survival (PFS) of the subject as compared to a reference PFS time.

269. The use of any one of embodiments 185-268, wherein the reference PFS time is the median PFS time of a population of subjects who have received a treatment comprising an anti-PD-L1 antagonist antibody without an anti-TIGIT antagonist antibody.

270. Use of an anti-TIGIT antagonist antibody and atezolizumab in the manufacture of a medicament for use in a method of treating a subject having a NSCLC, wherein the method comprises administering to the subject one or more dosing cycles of the medicament, and wherein the medicament is formulated for administration of the anti-TIGIT antagonist antibody at a fixed dose of 600 mg every three weeks and atezolizumab at a fixed dose of 1200 mg every three weeks, and wherein the anti-TIGIT antagonist antibody comprises:

a VH domain comprising the amino acid sequence of SEQ ID NO: 17 or 18; and

a VL domain comprising the amino acid sequence of SEQ ID NO: 19.

271. Use of tiragolumab and atezolizumab in the manufacture of a medicament for use in a method of treating a subject having a NSCLC, wherein the method comprises administering to the subject one or more dosing cycles of the medicament, and wherein the medicament is formulated for administration of tiragolumab at a fixed dose of 600 mg every three weeks and atezolizumab at a fixed dose of 1200 mg every three weeks.

272. A kit comprising an anti-TIGIT antagonist antibody, an anti-PD-L1 antagonist antibody, and a package insert comprising instructions to administer the anti-TIGIT antagonist antibody and the anti-PD-L1 antagonist antibody to a subject having a lung cancer in accordance with the methods of any one of embodiments 1-86 and 92-94.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method for treating a subject having a lung cancer, the method comprising administering to the subject one or more dosing cycles of an anti-TIGIT antagonist antibody at a fixed dose of between about 30 mg to about 1200 mg every three weeks and an anti-PD-L1 antagonist antibody at a fixed dose of between about 80 mg to about 1600 mg every three weeks.

2. The method of claim 1, wherein the method comprises administering to the subject an anti-TIGIT antagonist antibody at a fixed dose of between about 30 mg to about 600 mg every three weeks.

3. The method of claim 2, wherein the method comprises administering to the subject an anti-TIGIT antagonist antibody at a fixed dose of about 600 mg every three weeks.

4. The method of any one of claims 1-3, wherein the anti-TIGIT antagonist antibody comprises the following hypervariable regions (HVRs):
an HVR-H1 sequence comprising the amino acid sequence of SNSAAWN (SEQ ID NO: 1);
an HVR-H2 sequence comprising the amino acid sequence of KTYYRFKWYSDYAVSVKG (SEQ ID NO: 2);
an HVR-H3 sequence comprising the amino acid sequence of ESTTYDLLAGPFDY (SEQ ID NO: 3);
an HVR-L1 sequence comprising the amino acid sequence of KSSQTVLYSSNNKKYLA (SEQ ID NO: 4);
an HVR-L2 sequence comprising the amino acid sequence of WASTRES (SEQ ID NO: 5); and
an HVR-L3 sequence comprising the amino acid sequence of QQYYSTPFT (SEQ ID NO: 6).

5. The method of claim 4, wherein the anti-TIGIT antagonist antibody further comprises the following light chain variable region framework regions (FRs):
an FR-L1 comprising the amino acid sequence of DIVMTQSPDSLAVSLGERATINC (SEQ ID NO: 7);
an FR-L2 comprising the amino acid sequence of WYQQKPGQPPNLLIY (SEQ ID NO: 8);
an FR-L3 comprising the amino acid sequence of GVPDRFSGSGSGTDFTLTISSLQAEDVAVYYC (SEQ ID NO: 9); and
an FR-L4 comprising the amino acid sequence of FGPGTKVEIK (SEQ ID NO: 10).

6. The method of claim 4, wherein the anti-TIGIT antagonist antibody further comprises the following heavy chain variable region FRs:
an FR-H1 comprising the amino acid sequence of X₁VQLQQSGPGLVKPSQTLSLTCAISGDSVS (SEQ ID NO: 11), wherein X₁ is Q or E;
an FR-H2 comprising the amino acid sequence of WIRQSPSRGLEWLG (SEQ ID NO: 12);
an FR-H3 comprising the amino acid sequence of RITINPDTSKNQFSLQLNSVTPEDTAVFYCTR (SEQ ID NO: 13); and
an FR-H4 comprising the amino acid sequence of WGQGTLVTVSS (SEQ ID NO: 14).

7. The method of claim 6, wherein X₁ is Q.

8. The method of claim 6, wherein X₁ is E.

9. The method of any one of claims 4-8, wherein the anti-TIGIT antagonist antibody comprises:
(a) a heavy chain variable (VH) domain comprising an amino acid sequence having at least 95% sequence identity to the amino acid sequence of SEQ ID NO: 17 or 18;
(b) a light chain variable (VL) domain comprising an amino acid sequence having at least 95% sequence identity to the amino acid sequence of SEQ ID NO: 19; or
(c) a VH domain as in (a) and a VL domain as in (b).

10. The method of any one of claims 1-9, wherein the anti-TIGIT antagonist antibody comprises:
a VH domain comprising the amino acid sequence of SEQ ID NO: 17 or 18; and
a VL domain comprising the amino acid sequence of SEQ ID NO: 19.

11. The method of any one of claims 1-10, wherein the anti-TIGIT antagonist antibody is a monoclonal antibody.

12. The method of claim 11, wherein the anti-TIGIT antagonist antibody is a human antibody.

13. The method of any one of claims 1-12, wherein the anti-TIGIT antagonist antibody is a full-length antibody.

14. The method of any one of claims 1-6 and 8-13, wherein the anti-TIGIT antagonist antibody is tiragolumab.

15. The method of any one of claims 1-12, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

16. The method of any one of claims 1-15, wherein the anti-TIGIT antagonist antibody is an IgG class antibody.

17. The method of claim 16, wherein the IgG class antibody is an IgG1 subclass antibody.

18. The method of any one of claims 1-17, the method comprises administering to the subject an anti-PD-L1 antibody at a fixed dose of about 1200 mg every three weeks.

19. The method of any one of claims 1-18, wherein the anti-PD-L1 antagonist antibody is atezolizumab (MPDL3280A), YW243.55.S70, MSB0010718C, MDX-1105, or MEDI4736.

20. The method of claim 19, wherein the anti-PD-L1 antagonist antibody is atezolizumab.

21. The method of any one of claims 1-18, wherein the anti-PD-L1 antagonist antibody comprises the following HVRs:
an HVR-H1 sequence comprising the amino acid sequence of GFTFSDSWIH (SEQ ID NO: 20);
an HVR-H2 sequence comprising the amino acid sequence of AWISPYGGSTYYADSVKG (SEQ ID NO: 21);
an HVR-H3 sequence comprising the amino acid sequence of RHWPGGFDY (SEQ ID NO: 22);
an HVR-L1 sequence comprising the amino acid sequence of RASQDVSTAVA (SEQ ID NO: 23);
an HVR-L2 sequence comprising the amino acid sequence of SASFLYS (SEQ ID NO: 24); and
an HVR-L3 sequence comprising the amino acid sequence of QQYLYHPAT (SEQ ID NO: 25).

22. The method of claim 21, wherein the anti-PD-L1 antagonist antibody comprises:
(a) a heavy chain variable (VH) domain comprising an amino acid sequence having at least 95% sequence identity to the amino acid sequence of SEQ ID NO: 26;
(b) a light chain variable (VL) domain comprising an amino acid sequence having at least 95% sequence identity to the amino acid sequence of SEQ ID NO: 27; or
(c) a VH domain as in (a) and a VL domain as in (b).

23. The method of any one of claims 1-22, wherein the anti-PD-L1 antagonist antibody comprises:
a VH domain comprising the amino acid sequence of SEQ ID NO: 26; and
a VL domain comprising the amino acid sequence of SEQ ID NO: 27.

24. The method of any one of claims 21-23, wherein the anti-PD-L1 antagonist antibody is a monoclonal antibody.

25. The method of claim 24, wherein the anti-PD-L1 antagonist antibody is a humanized antibody.

26. The method of claim 24 or 25, wherein the anti-PD-L1 antagonist antibody is a full-length antibody.

27. The method of any one of claims 21-25, wherein the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

28. The method of any one of claims 21-27, wherein the anti-PD-L1 antagonist antibody is an IgG class antibody.

29. The method of claim 28, wherein the IgG class antibody is an IgG1 subclass antibody.

30. The method of any one of claims 1-29, wherein the method comprises administering to the subject the anti-TIGIT antagonist antibody at a fixed dose of about 600 mg every three weeks and the anti-PD-L1 antagonist antibody at a fixed dose of about 1200 mg every three weeks.

31. The method of any one of claims 1-30, wherein the length of each of the one or more dosing cycles is 21 days.

32. The method of any one of claims 1-31, wherein the method comprises administering to the subject the anti-TIGIT antagonist antibody and the anti-PD-L1 antagonist antibody on about Day 1 of each of the one or more dosing cycles.

33. The method of any one of claims 1-32, wherein the method comprises administering to the subject the anti-TIGIT antagonist antibody before the anti-PD-L1 antagonist antibody.

34. The method of claim 33, wherein the method comprises a first observation period following administration of the anti-TIGIT antagonist antibody and second observation period following administration of the anti-PD-L1 antagonist antibody.

35. The method of claim 34, wherein the first observation period and the second observation period are each between about 30 minutes to about 60 minutes in length.

36. The method of any one of claims 1-32, wherein the method comprises administering to the subject the anti-PD-L1 antagonist antibody before the anti-TIGIT antagonist antibody.

37. The method of claim 36, wherein the method comprises a first observation period following administration of the anti-PD-L1 antagonist antibody and second observation period following administration of the anti-TIGIT antagonist antibody.

38. The method of claim 37, wherein the first observation period and the second observation period are each between about 30 minutes to about 60 minutes in length.

39. The method of any one of claims 1-32, wherein the method comprises administering to the subject the anti-TIGIT antagonist antibody and the anti-PD-L1 antagonist antibody simultaneously.

40. The method of any one of claims 1-39, wherein the method comprises administering to the subject the anti-TIGIT antagonist antibody and anti-PD-L1 antagonist antibody intravenously.

41. The method of claim 40, wherein the method comprises administering to the subject the anti-TIGIT antagonist antibody by intravenous infusion over 60±10 minutes.

42. The method of claim 40 or 41, wherein the method comprises administering to the subject the anti-PD-L1 antagonist antibody by intravenous infusion over 60±15 minutes.

43. The method of any one of claims 1-42, wherein a tumor sample obtained from the subject has been determined to have a detectable expression level of PD-L1.

44. The method of claim 43, wherein the detectable expression level of PD-L1 is a detectable protein expression level of PD-L1.

45. The method of claim 44, wherein the detectable protein expression level of PD-L1 has been determined by an immunohistochemical (IHC) assay.

46. The method of claim 45, wherein the IHC assay uses anti-PD-L1 antibody 22C3, SP142, SP263, or 28-8.

47. The method of claim 46, wherein the IHC assay uses anti-PD-L1 antibody 22C3.

48. The method of claim 47, wherein the tumor sample has been determined to have a tumor proportion score (TPS) of greater than, or equal to, 1%.

49. The method of claim 48, wherein the TPS is greater than, or equal to, 1% and less than 50%.

50. The method of claim 48, wherein the TPS is greater than, or equal to, 50%.

51. The method of claim 46, wherein the IHC assay uses anti-PD-L1 antibody SP142.

52. The method of claim 51, wherein the tumor sample has been determined to have a detectable expression level of PD-L1 in greater than, or equal to, 1% of the tumor cells in the tumor sample.

53. The method of claim 52, wherein the tumor sample has been determined to have a detectable expression level of PD-L1 in greater than, or equal to, 1% and less than 5% of the tumor cells in the tumor sample.

54. The method of claim 52, wherein the tumor sample has been determined to have a detectable expression level of PD-L1 in greater than, or equal to, 5% and less than 50% of the tumor cells in the tumor sample.

55. The method of claim 52, wherein the tumor sample has been determined to have a detectable expression level of PD-L1 in greater than, or equal to, 50% of the tumor cells in the tumor sample.

56. The method of any one of claims 51-55, wherein the tumor sample has been determined to have a detectable expression level of PD-L1 in tumor-infiltrating immune cells that comprise greater than, or equal to, 1% of the tumor sample.

57. The method of claim 56, wherein the tumor sample has been determined to have a detectable expression level of PD-L1 in tumor-infiltrating immune cells that comprise greater than, or equal to, 1% and less than 5% of the tumor sample.

58. The method of claim 56, wherein the tumor sample has been determined to have a detectable expression level of PD-L1 in tumor-infiltrating immune cells that comprise greater than, or equal to, 5% and less than 10% of the tumor sample.

59. The method of claim 56, wherein the tumor sample has been determined to have a detectable expression level of PD-L1 in tumor-infiltrating immune cells that comprise greater than, or equal to, 10% of the tumor sample.

60. The method of claim 43, wherein the detectable expression level of PD-L1 is a detectable nucleic acid expression level of PD-L1.

61. The method of claim 60, wherein the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

62. The method of any one of claims 1-61, wherein the lung cancer is a non-small cell lung cancer (NSCLC).

63. The method of claim 62, wherein the NSCLC is a squamous NSCLC.

64. The method of claim 62, wherein the NSCLC is a non-squamous NSCLC.

65. The method of any one of claims 62-64, wherein the NSCLC is a locally advanced unresectable NSCLC.

66. The method of claim 65, wherein the NSCLC is a Stage IIIB NSCLC.

67. The method of any one of claims 62-64, wherein the NSCLC is a recurrent or metastatic NSCLC.

68. The method of claim 67, wherein the NSCLC is a Stage IV NSCLC.

69. The method of claim 67 or 68, wherein the subject has not been previously treated for Stage IV NSCLC.

70. The method of any one of claims 1-69, wherein the subject does not have a sensitizing epidermal growth factor receptor (EGFR) gene mutation or anaplastic lymphoma kinase (ALK) gene rearrangement.

71. The method of any one of claims 1-70, wherein the subject does not have a pulmonary lymphoepithelioma-like carcinoma subtype of NSCLC.

72. The method of any one of claims 1-71, wherein the subject does not have an active Epstein-Barr virus (EBV) infection or a known or suspected chronic active EBV infection.

73. The method of any one of claims 1-72, wherein the subject is negative for EBV IgM or negative by EBV PCR.

74. The method of claim 73, wherein the subject is negative for EBV IgM and negative by EBV PCR.

75. The method of claim 73 or 74, wherein the subject is positive for EBV IgG or positive for Epstein-Barr nuclear antigen (EBNA).

76. The method of claim 75, wherein the subject is positive for EBV IgG and positive for EBNA.

77. The method of any one of claims 1-74, wherein the subject is negative for EBV IgG or negative for EBNA.

78. The method of claim 77, wherein the subject is negative for EBV IgG and negative for EBNA.

79. The method of any one of claims 1-78, wherein the treating results in a clinical response.

80. The method of claim 79, wherein the clinical response is an increase in the objective response rate (ORR) of the subject as compared to a reference ORR.

81. The method of claim 80, wherein the reference ORR is the median ORR of a population of subjects who have received a treatment comprising an anti-PD-L1 antagonist antibody without an anti-TIGIT antagonist antibody.

82. The method of any one of claims 79-81, wherein the clinical response is an increase in the progression-free survival (PFS) of the subject as compared to a reference PFS time.

83. The method of any one of claims 79-82, wherein the reference PFS time is the median PFS time of a population of subjects who have received a treatment comprising an anti-PD-L1 antagonist antibody without an anti-TIGIT antagonist antibody.

84. A method for treating a subject having a NSCLC, the method comprising administering to the subject one or more dosing cycles of an anti-TIGIT antagonist antibody at a fixed dose of 600 mg every three weeks and atezolizumab at a fixed dose of 1200 mg every three weeks, wherein the anti-TIGIT antagonist antibody comprises:
a VH domain comprising the amino acid sequence of SEQ ID NO: 17 or 18; and
a VL domain comprising the amino acid sequence of SEQ ID NO: 19.

85. A method of treating a subject having a NSCLC, the method comprising:
(a) obtaining a tumor sample from the subject;
(b) detecting the protein expression level of PD-L1 in the tumor sample by an IHC assay using anti-PD-L1 antibody 22C3 and determining a TPS therefrom;
(c) identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1% and less than 50%, wherein the anti-TIGIT antagonist antibody comprises:
a VH domain comprising the amino acid sequence of SEQ ID NO: 17 or 18; and
a VL domain comprising the amino acid sequence of SEQ ID NO: 19; and
(d) administering to the identified subject the therapy.

86. A method of treating a subject having a NSCLC, the method comprising:
(a) obtaining a tumor sample from the subject;
(b) detecting the protein expression level of PD-L1 in the tumor sample by an IHC assay using anti-PD-L1 antibody 22C3 and determining a TPS therefrom;
(c) identifying the subject as one who is likely to benefit from a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 50%, wherein the anti-TIGIT antagonist antibody comprises:
a VH domain comprising the amino acid sequence of SEQ ID NO: 17 or 18; and
a VL domain comprising the amino acid sequence of SEQ ID NO: 19; and
(d) administering to the identified subject the therapy.

87. A method of selecting a therapy for a subject having a NSCLC, the method comprising:
(a) determining a TPS from a tumor sample from the subject by an IHC assay using anti-PD-L1 antibody 22C3; and
(b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 1% and less than 50%, wherein the anti-TIGIT antagonist antibody comprises:
a VH domain comprising the amino acid sequence of SEQ ID NO: 17 or 18; and
a VL domain comprising the amino acid sequence of SEQ ID NO: 19.

88. A method of selecting a therapy for a subject having a NSCLC, the method comprising:
(a) determining a TPS from a tumor sample from the subject by an IHC assay using anti-PD-L1 antibody 22C3; and
(b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks based on the TPS having been determined to be greater than, or equal to, 50%, wherein the anti-TIGIT antagonist antibody comprises:
a VH domain comprising the amino acid sequence of SEQ ID NO: 17 or 18; and
a VL domain comprising the amino acid sequence of SEQ ID NO: 19.

89. A method of selecting a therapy for a subject having a NSCLC, the method comprising:
(a) detecting the mutational status of the epidermal growth factor receptor (EGFR) gene and anaplastic lymphoma kinase (ALK) gene from a sample from the subject and detecting the absence of a sensitizing EGFR gene mutation or ALK gene rearrangement; and
(b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks, based on the subject not having a sensitizing EGFR gene mutation or ALK gene rearrangement, wherein the anti-TIGIT antagonist antibody comprises:
a VH domain comprising the amino acid sequence of SEQ ID NO: 17 or 18; and
a VL domain comprising the amino acid sequence of SEQ ID NO: 19.

90. A method of selecting a therapy for a subject having a NSCLC, the method comprising:
(a) biopsying a tumor sample from the subject and detecting a subtype of the NSCLC other than a pulmonary lymphoepithelioma-like carcinoma; and
(b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks, based on the subject not having a pulmonary lymphoepithelioma-like carcinoma subtype of NSCLC, wherein the anti-TIGIT antagonist antibody comprises:
a VH domain comprising the amino acid sequence of SEQ ID NO: 17 or 18; and
a VL domain comprising the amino acid sequence of SEQ ID NO: 19.

91. A method of selecting a therapy for a subject having a NSCLC, the method comprising:
(a) detecting the presence of one or more of Epstein-Barr virus (EBV) IgM, EBV IgG, Epstein-Barr nuclear antigen (EBNA), and Epstein-Barr viral particles in a sample from the subject, and
(b) selecting for the subject a therapy comprising one or more dosing cycles of an anti-TIGIT antagonist antibody administered at a fixed dose of 600 mg every three weeks and atezolizumab administered at a fixed dose of 1200 mg every three weeks, on based on the subject being:
(i) negative for EBV IgG and/or EBNA; or
(ii) positive for EBV IgG and/or EBNA, and negative for both EBV IgM and Epstein-Barr viral particles,
wherein the anti-TIGIT antagonist antibody comprises:
a VH domain comprising the amino acid sequence of SEQ ID NO: 17 or 18; and
a VL domain comprising the amino acid sequence of SEQ ID NO: 19.

92. A method for treating a subject having a NSCLC, the method comprising administering to the subject one or more dosing cycles of tiragolumab at a fixed dose of 600 mg every three weeks and atezolizumab at a fixed dose of 1200 mg every three weeks.

93. A method of treating a subject having a NSCLC, the method comprising:
(a) obtaining a tumor sample from the subject;
(b) detecting the protein expression level of PD-L1 in the tumor sample by an IHC assay using anti-PD-L1 antibody 22C3 and determining a TPS therefrom;
(c) identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1% and less than 50%; and
(d) administering to the identified subject the therapy.

94. A method of treating a subject having a NSCLC, the method comprising:
(a) obtaining a tumor sample from the subject;
(b) detecting the protein expression level of PD-L1 in the tumor sample by an IHC assay using anti-PD-L1 antibody 22C3 and determining a TPS therefrom;
(c) identifying the subject as one who is likely to benefit from a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 50%; and
(d) administering to the identified subject the therapy.

95. A method of selecting a therapy for a subject having a NSCLC, the method comprising:
(a) determining a TPS from a tumor sample from the subject by an IHC assay using anti-PD-L1 antibody 22C3; and
(b) selecting for the subject a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 1% and less than 50%.

96. A method of selecting a therapy for a subject having a NSCLC, the method comprising:
(a) determining a TPS from a tumor sample from the subject by an IHC assay using anti-PD-L1 antibody 22C3; and
(b) selecting for the subject a therapy comprising one or more dosing cycles of tiragolumab administered at a fixed dose of 600 mg every three weeks and atezolizumab administered at a fixed dose of 1200 mg every three weeks based on the TPS having been determined to be greater than, or equal to, 50%.

97. A method of selecting a therapy for a subject having a NSCLC, the method comprising:
(a) detecting the mutational status of the epidermal growth factor receptor (EGFR) gene and anaplastic lymphoma kinase (ALK) gene from a sample from the subject and detecting the absence of a sensitizing EGFR gene mutation or ALK gene rearrangement; and
(b) selecting for the subject a therapy comprising one or more dosing cycles of tiragolumab administered at a fixed dose of 600 mg every three weeks and atezolizumab administered at a fixed dose of 1200 mg every three weeks, based on the subject not having a sensitizing EGFR gene mutation or ALK gene rearrangement.

98. A method of selecting a therapy for a subject having a NSCLC, the method comprising:
(a) biopsying a tumor sample from the subject and detecting a subtype of the NSCLC other than a pulmonary lymphoepithelioma-like carcinoma; and
(b) selecting for the subject a therapy comprising one or more dosing cycles of tiragolumab administered at a fixed dose of 600 mg every three weeks and atezolizumab administered at a fixed dose of 1200 mg every three weeks, based on the subject not having a pulmonary lymphoepithelioma-like carcinoma subtype of NSCLC.

99. A method of selecting a therapy for a subject having a NSCLC, the method comprising:
(a) detecting the presence of one or more of Epstein-Barr virus (EBV) IgM, EBV IgG, Epstein-Barr nuclear antigen (EBNA), and Epstein-Barr viral particles in a sample from the subject, and
(b) selecting for the subject a therapy comprising one or more dosing cycles of tiragolumab administered at a fixed dose of 600 mg every three weeks and atezolizumab administered at a fixed dose of 1200 mg every three weeks, on based on the subject being:
(i) negative for EBV IgG and/or EBNA; or
(ii) positive for EBV IgG and/or EBNA, and negative for both EBV IgM and Epstein-Barr viral particles.

100. An anti-TIGIT antagonist antibody and an anti-PD-L1 antagonist antibody for use in a method of treating a subject having a lung cancer, wherein the method comprises administering to the subject one or more dosing cycles of the anti-TIGIT antagonist antibody at a fixed dose of between about 30 mg to about 1200 mg every three weeks and the anti-PD-L1 antagonist antibody at a fixed dose of between about 80 mg to about 1600 mg every three weeks.

101. The anti-TIGIT antagonist antibody and anti-PD-L1 antagonist antibody for use of claim 100, wherein the anti-TIGIT antagonist antibody is to be administered to the subject at a fixed dose of between about 30 mg to about 600 mg every three weeks.

102. The anti-TIGIT antagonist antibody and anti-PD-L1 antagonist antibody for use of claim 101, wherein the anti-TIGIT antagonist antibody is to be administered to the subject at a fixed dose of about 600 mg every three weeks.

103. The anti-TIGIT antagonist antibody and anti-PD-L1 antagonist antibody for use of any one of claims 100-102, wherein the anti-TIGIT antagonist antibody comprises the following HVRs:
an HVR-H1 sequence comprising the amino acid sequence of SNSAAWN (SEQ ID NO: 1);
an HVR-H2 sequence comprising the amino acid sequence of KTYYRFKWYSDYAVSVKG (SEQ ID NO: 2);
an HVR-H3 sequence comprising the amino acid sequence of ESTTYDLLAGPFDY (SEQ ID NO: 3);
an HVR-L1 sequence comprising the amino acid sequence of KSSQTVLYSSNNKKYLA (SEQ ID NO: 4);
an HVR-L2 sequence comprising the amino acid sequence of WASTRES (SEQ ID NO: 5); and
an HVR-L3 sequence comprising the amino acid sequence of QQYYSTPFT (SEQ ID NO: 6).

104. The anti-TIGIT antagonist antibody and anti-PD-L1 antagonist antibody for use of claim 103, wherein the anti-TIGIT antagonist antibody further comprises the following light chain variable region FRs:
an FR-L1 comprising the amino acid sequence of DIVMTQSPDSLAVSLGERATINC (SEQ ID NO: 7);
an FR-L2 comprising the amino acid sequence of WYQQKPGQPPNLLIY (SEQ ID NO: 8);
an FR-L3 comprising the amino acid sequence of GVPDRFSGSGSGTDFTLTISSLQAEDVAVYYC (SEQ ID NO: 9); and
an FR-L4 comprising the amino acid sequence of FGPGTKVEIK (SEQ ID NO: 10).

105. The anti-TIGIT antagonist antibody and anti-PD-L1 antagonist antibody for use of claim 103, wherein the anti-TIGIT antagonist antibody further comprises the following heavy chain variable region FRs:
an FR-H1 comprising the amino acid sequence of X₁VQLQQSGPGLVKPSQTLSLTCAISGDSVS (SEQ ID NO: 11), wherein X₁ is Q or E;
an FR-H2 comprising the amino acid sequence of WIRQSPSRGLEWLG (SEQ ID NO: 12);
an FR-H3 comprising the amino acid sequence of RITINPDTSKNQFSLQLNSVTPEDTAVFYCTR (SEQ ID NO: 13); and
an FR-H4 comprising the amino acid sequence of WGQGTLVTVSS (SEQ ID NO: 14).

106. The anti-TIGIT antagonist antibody and anti-PD-L1 antagonist antibody for use of claim 105, wherein X₁ is Q.

107. The anti-TIGIT antagonist antibody and anti-PD-L1 antagonist antibody of claim 105, wherein X₁ is E.

108. The anti-TIGIT antagonist antibody and anti-PD-L1 antagonist antibody for use of any one of claims 103-107, wherein the anti-TIGIT antagonist antibody comprises:
(a) a heavy chain variable (VH) domain comprising an amino acid sequence having at least 95% sequence identity to the amino acid sequence of SEQ ID NO: 17 or 18;
(b) a light chain variable (VL) domain comprising an amino acid sequence having at least 95% sequence identity to the amino acid sequence of SEQ ID NO: 19; or
(c) a VH domain as in (a) and a VL domain as in (b).

109. The anti-TIGIT antagonist antibody and anti-PD-L1 antagonist antibody for use of any one of claims 100-108, wherein the anti-TIGIT antagonist antibody comprises:
a VH domain comprising the amino acid sequence of SEQ ID NO: 17 or 18; and
a VL domain comprising the amino acid sequence of SEQ ID NO: 19.

110. The anti-TIGIT antagonist antibody and anti-PD-L1 antagonist antibody for use of any one of claims 100-109, wherein the anti-TIGIT antagonist antibody is a monoclonal antibody.

111. The anti-TIGIT antagonist antibody and anti-PD-L1 antagonist antibody for use of claim 110, wherein the anti-TIGIT antagonist antibody is a human antibody.

112. The anti-TIGIT antagonist antibody and anti-PD-L1 antagonist antibody for use of any one of claims 100-111, wherein the anti-TIGIT antagonist antibody is a full-length antibody.

113. The anti-TIGIT antagonist antibody and anti-PD-L1 antagonist antibody for use of any one of claims 100-105 and 107-112, wherein the anti-TIGIT antagonist antibody is tiragolumab.

114. The anti-TIGIT antagonist antibody and anti-PD-L1 antagonist antibody for use of any one of claims 100-111, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

115. The anti-TIGIT antagonist antibody and anti-PD-L1 antagonist antibody for use of any one of claims 100-114, wherein the anti-TIGIT antagonist antibody is an IgG class antibody.

116. The anti-TIGIT antagonist antibody and anti-PD-L1 antagonist antibody for use of claim 115, wherein the IgG class antibody is an IgG1 subclass antibody.

117. The anti-TIGIT antagonist antibody and anti-PD-L1 antagonist antibody for use of any one of claims 100-116, wherein the anti-PD-L1 antagonist antibody is to be administered to the subject at a fixed dose of about 1200 mg every three weeks.

118. The anti-TIGIT antagonist antibody and anti-PD-L1 antagonist antibody for use of any one of claims 100-117, wherein the anti-PD-L1 antagonist antibody is atezolizumab (MPDL3280A), YW243.55.S70, MSB0010718C, MDX-1105, or MEDI4736.

119. The anti-TIGIT antagonist antibody and anti-PD-L1 antagonist antibody for use of claim 118, wherein the anti-PD-L1 antagonist antibody is atezolizumab.

120. The anti-TIGIT antagonist antibody and anti-PD-L1 antagonist antibody for use of any one of claims 100-117, wherein the anti-PD-L1 antagonist antibody comprises the following HVRs:
an HVR-H1 sequence comprising the amino acid sequence of GFTFSDSWIH (SEQ ID NO: 20);
an HVR-H2 sequence comprising the amino acid sequence of AWISPYGGSTYYADSVKG (SEQ ID NO: 21);
an HVR-H3 sequence comprising the amino acid sequence of RHWPGGFDY (SEQ ID NO: 22);
an HVR-L1 sequence comprising the amino acid sequence of RASQDVSTAVA (SEQ ID NO: 23);
an HVR-L2 sequence comprising the amino acid sequence of SASFLYS (SEQ ID NO: 24); and
an HVR-L3 sequence comprising the amino acid sequence of QQYLYHPAT (SEQ ID NO: 25).

121. The anti-TIGIT antagonist antibody and anti-PD-L1 antagonist antibody for use of claim 120, wherein the anti-PD-L1 antagonist antibody comprises:
(a) a heavy chain variable (VH) domain comprising an amino acid sequence having at least 95% sequence identity to the amino acid sequence of SEQ ID NO: 26;
(b) a light chain variable (VL) domain comprising an amino acid sequence having at least 95% sequence identity to the amino acid sequence of SEQ ID NO: 27; or
(c) a VH domain as in (a) and a VL domain as in (b).

122. The anti-TIGIT antagonist antibody and anti-PD-L1 antagonist antibody for use of any one of claims 100-121, wherein the anti-PD-L1 antagonist antibody comprises:
a VH domain comprising the amino acid sequence of SEQ ID NO: 26; and
a VL domain comprising the amino acid sequence of SEQ ID NO: 27.

123. The anti-TIGIT antagonist antibody and anti-PD-L1 antagonist antibody for use of any one of claims 120-122, wherein the anti-PD-L1 antagonist antibody is a monoclonal antibody.

124. The anti-TIGIT antagonist antibody and anti-PD-L1 antagonist antibody for use of claim 123, wherein the anti-PD-L1 antagonist antibody is a humanized antibody.

125. The anti-TIGIT antagonist antibody and anti-PD-L1 antagonist antibody for use of claim 123 or 124, wherein the anti-PD-L1 antagonist antibody is a full-length antibody.

126. The anti-TIGIT antagonist antibody and anti-PD-L1 antagonist antibody for use of any one of claims 120-124, wherein the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

127. The anti-TIGIT antagonist antibody and anti-PD-L1 antagonist antibody for use of any one of claims 120-126, wherein the anti-PD-L1 antagonist antibody is an IgG class antibody.

128. The anti-TIGIT antagonist antibody and anti-PD-L1 antagonist antibody for use of claim 127, wherein the IgG class antibody is an IgG1 subclass antibody.

129. The anti-TIGIT antagonist antibody and anti-PD-L1 antagonist antibody for use of any one of claims 100-128, wherein the anti-TIGIT antagonist antibody is to be administered to the subject at a fixed dose of about 600 mg every three weeks and the anti-PD-L1 antagonist antibody is to be administered to the subject at a fixed dose of about 1200 mg every three weeks.

130. The anti-TIGIT antagonist antibody and anti-PD-L1 antagonist antibody for use of any one of claims 100-129, wherein the length of each of the one or more dosing cycles is 21 days.

131. The anti-TIGIT antagonist antibody and anti-PD-L1 antibody for use of any one of claims 100-130, wherein the anti-TIGIT antagonist antibody and anti-PD-L1 antagonist antibody are to be administered to the subject on about Day 1 of each of the one or more dosing cycles.

132. The anti-TIGIT antagonist antibody and anti-PD-L1 antagonist antibody for use of any one of claims 100-131, wherein the anti-TIGIT antagonist antibody is to be administered to the subject before the anti-PD-L1 antagonist antibody.

133. The anti-TIGIT antagonist antibody and anti-PD-L1 antagonist antibody for use of claim 132, wherein a first observation period is to follow administration of the anti-TIGIT antagonist antibody and second observation period is to follow administration of the anti-PD-L1 antagonist antibody.

134. The anti-TIGIT antagonist antibody and anti-PD-L1 antagonist antibody for use of claim 133, wherein the first observation period and the second observation period are each between about 30 minutes to about 60 minutes in length.

135. The anti-TIGIT antagonist antibody and anti-PD-L1 antagonist antibody for use of any one of claims 100-131, wherein the anti-PD-L1 antagonist antibody is to be administered to the subject before the anti-TIGIT antagonist antibody.

136. The anti-TIGIT antagonist antibody and anti-PD-L1 antagonist antibody for use of claim 135, wherein a first observation period is to follow administration of the anti-PD-L1 antagonist antibody and second observation period is to follow administration of the anti-TIGIT antagonist antibody.

137. The anti-TIGIT antagonist antibody and anti-PD-L1 antagonist antibody for use of claim 136, wherein the first observation period and the second observation period are each between about 30 minutes to about 60 minutes in length.

138. The anti-TIGIT antagonist antibody and anti-PD-L1 antagonist antibody for use of any one of claims 100-131, wherein the anti-TIGIT antagonist antibody is to be administered to the subject simultaneously with the anti-PD-L1 antagonist antibody.

139. The anti-TIGIT antagonist antibody and anti-PD-L1 antagonist antibody for use of any one of claims 100-138, wherein the anti-TIGIT antagonist antibody and anti-PD-L1 antagonist antibody are to be administered to the subject intravenously.

140. The anti-TIGIT antagonist antibody and anti-PD-L1 antagonist antibody for use of claim 139, wherein the anti-TIGIT antagonist antibody is to be administered to the subject by intravenous infusion over 60±10 minutes.

141. The anti-TIGIT antagonist antibody and anti-PD-L1 antagonist antibody for use of claim 139 or 140, wherein the anti-PD-L1 antagonist antibody is to be administered to the subject by intravenous infusion over 60±15 minutes.

142. The anti-TIGIT antagonist antibody and anti-PD-L1 antagonist antibody for use of any one of claims 100-141, wherein a tumor sample obtained from the subject has been determined to have a detectable expression level of PD-L1.

143. The anti-TIGIT antagonist antibody and anti-PD-L1 antibody for use of claim 142, wherein the detectable expression level of PD-L1 is a detectable protein expression level of PD-L1.

144. The anti-TIGIT antagonist antibody and anti-PD-L1 antagonist antibody for use of claim 143, wherein the detectable protein expression level of PD-L1 has been determined by an immunohistochemical (IHC) assay.

145. The anti-TIGIT antagonist antibody and anti-PD-L1 antagonist antibody for use of claim 144, wherein the IHC assay uses anti-PD-L1 antibody 22C3, SP142, SP263, or 28-8.

146. The anti-TIGIT antagonist antibody and anti-PD-L1 antagonist antibody for use of claim 145, wherein the IHC assay uses anti-PD-L1 antibody 22C3.

147. The anti-TIGIT antagonist antibody and anti-PD-L1 antagonist antibody for use of claim 146, wherein the tumor sample has been determined to have a tumor proportion score (TPS) of greater than, or equal to, 1%.

148. The anti-TIGIT antagonist antibody and anti-PD-L1 antagonist antibody for use of claim 147, wherein the TPS is greater than, or equal to, 1% and less than 50%.

149. The anti-TIGIT antagonist antibody and anti-PD-L1 antagonist antibody for use of claim 147, wherein the TPS is greater than, or equal to, 50%.

150. The anti-TIGIT antagonist antibody and anti-PD-L1 antagonist antibody for use of claim 145, wherein the IHC assay uses anti-PD-L1 antibody SP142.

151. The anti-TIGIT antagonist antibody and anti-PD-L1 antagonist antibody for use of claim 150, wherein the tumor sample has been determined to have a detectable expression level of PD-L1 in greater than, or equal to, 1% of the tumor cells in the tumor sample.

152. The anti-TIGIT antagonist antibody and anti-PD-L1 antagonist antibody for use of claim 151, wherein the tumor sample has been determined to have a detectable expression level of PD-L1 in greater than, or equal to, 1% and less than 5% of the tumor cells in the tumor sample.

153. The anti-TIGIT antagonist antibody and anti-PD-L1 antagonist antibody for use of claim 151, wherein the tumor sample has been determined to have a detectable expression level of PD-L1 in greater than, or equal to, 5% and less than 50% of the tumor cells in the tumor sample.

154. The anti-TIGIT antagonist antibody and anti-PD-L1 antagonist antibody for use of claim 151, wherein the tumor sample has been determined to have a detectable expression level of PD-L1 in greater than, or equal to, 50% of the tumor cells in the tumor sample.

155. The anti-TIGIT antagonist antibody and anti-PD-L1 antagonist antibody for use of any one of claims 150-154, wherein the tumor sample has been determined to have a detectable expression level of PD-L1 in tumor-infiltrating immune cells that comprise greater than, or equal to, 1% of the tumor sample.

156. The anti-TIGIT antagonist antibody and anti-PD-L1 antagonist antibody for use of claim 155, wherein the tumor sample has been determined to have a detectable expression level of PD-L1 in tumor-infiltrating immune cells that comprise greater than, or equal to, 1% and less than 5% of the tumor sample.

157. The anti-TIGIT antagonist antibody and anti-PD-L1 antagonist antibody for use of claim 155, wherein the tumor sample has been determined to have a detectable expression level of PD-L1 in tumor-infiltrating immune cells that comprise greater than, or equal to, 5% and less than 10% of the tumor sample.

158. The anti-TIGIT antagonist antibody and anti-PD-L1 antagonist antibody for use of claim 155, wherein the tumor sample has been determined to have a detectable expression level of PD-L1 in tumor-infiltrating immune cells that comprise greater than, or equal to, 10% of the tumor sample.

159. The anti-TIGIT antagonist antibody and anti-PD-L1 antagonist antibody for use of claim 142, wherein the detectable expression level of PD-L1 is a detectable nucleic acid expression level of PD-L1.

160. The anti-TIGIT antagonist antibody and anti-PD-L1 antagonist antibody for use of claim 159, wherein the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

161. The anti-TIGIT antagonist antibody and anti-PD-L1 antagonist antibody for use of any one of claims 100-160, wherein the lung cancer is a non-small cell lung cancer (NSCLC).

162. The anti-TIGIT antagonist antibody and anti-PD-L1 antagonist antibody for use of claim 161, wherein the NSCLC is a squamous NSCLC.

163. The anti-TIGIT antagonist antibody and anti-PD-L1 antagonist antibody for use of claim 161, wherein the NSCLC is a non-squamous NSCLC.

164. The anti-TIGIT antagonist antibody and anti-PD-L1 antagonist antibody for use of any one of claims 161-163, wherein the NSCLC is a locally advanced unresectable NSCLC.

165. The anti-TIGIT antagonist antibody and anti-PD-L1 antagonist antibody for use of claim 164, wherein the NSCLC is a Stage IIIB NSCLC.

166. The anti-TIGIT antagonist antibody and anti-PD-L1 antagonist antibody for use of any one of claims 161-163, wherein the NSCLC is a recurrent or metastatic NSCLC.

167. The anti-TIGIT antagonist antibody and anti-PD-L1 antagonist antibody for use of claim 166, wherein the NSCLC is a Stage IV NSCLC.

168. The anti-TIGIT antagonist antibody and anti-PD-L1 antagonist antibody for use of claim 166 or 167, wherein the subject has not been previously treated for Stage IV NSCLC.

169. The anti-TIGIT antagonist antibody and anti-PD-L1 antagonist antibody for use of any one of claims 100-168, wherein the subject does not have a sensitizing epidermal growth factor receptor (EGFR) gene mutation or anaplastic lymphoma kinase (ALK) gene rearrangement.

170. The anti-TIGIT antagonist antibody and anti-PD-L1 antagonist antibody for use of any one of claims 100-169, wherein the subject does not have a pulmonary lymphoepithelioma-like carcinoma subtype of NSCLC.

171. The anti-TIGIT antagonist antibody and anti-PD-L1 antagonist antibody for use of any one of claims 100-170, wherein the subject does not have an active EBV infection or a known or suspected chronic active EBV infection.

172. The anti-TIGIT antagonist antibody and anti-PD-L1 antagonist antibody for use of any one of claims 100-171, wherein the subject is negative for EBV IgM or negative by EBV PCR.

173. The anti-TIGIT antagonist antibody and anti-PD-L1 antagonist antibody for use of claim 172, wherein the subject is negative for EBV IgM and negative by EBV PCR.

174. The anti-TIGIT antagonist antibody and anti-PD-L1 antagonist antibody for use of claim 172 or 173, wherein the subject is positive for EBV IgG or positive for EBNA.

175. The anti-TIGIT antagonist antibody and anti-PD-L1 antagonist antibody for use of claim 174, wherein the subject is positive for EBV IgG and positive for EBNA.

176. The anti-TIGIT antagonist antibody and anti-PD-L1 antagonist antibody for use of any one of claims 100-173, wherein the subject is negative for EBV IgG or negative for EBNA.

177. The anti-TIGIT antagonist antibody and anti-PD-L1 antagonist antibody for use of claim 176, wherein the subject is negative for EBV IgG and negative for EBNA.

178. The anti-TIGIT antagonist antibody and anti-PD-L1 antagonist antibody for use of any one of claims 100-177, wherein administration of the anti-TIGIT antagonist antibody and anti-PD-L1 antagonist antibody results in a clinical response.

179. The anti-TIGIT antagonist antibody and anti-PD-L1 antagonist antibody for use of claim 178, wherein the clinical response is an increase in the objective response rate (ORR) of the subject as compared to a reference ORR.

180. The anti-TIGIT antagonist antibody and anti-PD-L1 antagonist antibody for use of claim 179, wherein the reference ORR is the median ORR of a population of subjects who have received a treatment comprising an anti-PD-L1 antagonist antibody without an anti-TIGIT antagonist antibody.

181. The anti-TIGIT antagonist antibody and anti-PD-L1 antagonist antibody for use of any one of claims 178-180, wherein the clinical response is an increase in the progression-free survival (PFS) of the subject as compared to a reference PFS time.

182. The anti-TIGIT antagonist antibody and anti-PD-L1 antagonist antibody for use of any one of claims 178-181, wherein the reference PFS time is the median PFS time of a population of subjects who have received a treatment comprising an anti-PD-L1 antagonist antibody without an anti-TIGIT antagonist antibody.

183. An anti-TIGIT antagonist antibody and atezolizumab for use in a method of treating a subject having a NSCLC, wherein the method comprises administering to the subject one or more dosing cycles of an anti-TIGIT antagonist antibody at a fixed dose of 600 mg every three weeks and atezolizumab at a fixed dose of 1200 mg every three weeks, wherein the anti-TIGIT antagonist antibody comprises:
a VH domain comprising the amino acid sequence of SEQ ID NO: 17 or 18; and
a VL domain comprising the amino acid sequence of SEQ ID NO: 19.

184. Tiragolumab and atezolizumab for use in a method of treating a subject having a NSCLC, wherein the method comprises administering to the subject one or more dosing cycles of tiragolumab at a fixed dose of 600 mg every three weeks and atezolizumab at a fixed dose of 1200 mg every three weeks.

185. Use of an anti-TIGIT antagonist antibody and an anti-PD-L1 antagonist antibody in the manufacture of a medicament for use in a method of treating a subject having a lung cancer, wherein the method comprises administering to the subject one or more dosing cycles of the medicament, and wherein the medicament is formulated for administration of the anti-TIGIT antagonist antibody at a fixed dose of between about 30 mg to about 1200 mg every three weeks and the anti-PD-L1 antagonist antibody at a fixed dose of between about 80 mg to about 1600 mg every three weeks.

186. Use of an anti-TIGIT antagonist antibody in the manufacture of a medicament for use in a method of treating a subject having a lung cancer, wherein the method comprises administering to the subject one or more dosing cycles of the medicament and an anti-PD-L1 antagonist antibody, and wherein the medicament is formulated for administration of the anti-TIGIT antagonist antibody at a fixed dose of between about 30 mg to about 1200 mg every three weeks and the anti-PD-L1 antagonist antibody is to be administered at a fixed dose of between about 80 mg to about 1600 mg every three weeks.

187. Use of an anti-PD-L1 antagonist antibody in the manufacture of a medicament for use in a method of treating a subject having a lung cancer, wherein the method comprises administering to the subject one or more dosing cycles of the medicament and an anti-TIGIT antagonist antibody, and wherein the medicament is formulated for administration of the anti-PD-L1 antagonist antibody at a fixed dose of between about 80 mg to about 1600 mg every three weeks and the anti-TIGIT antagonist antibody is to be administered at a fixed dose of between about 30 mg to about 1200 mg every three weeks.

188. The use of any one of claims 185-187, wherein the anti-TIGIT antagonist antibody is to be administered to the subject at a fixed dose of between about 30 mg to about 600 mg every three weeks.

189. The use of claim 188, wherein the anti-TIGIT antagonist antibody is to be administered to the subject at a fixed dose of about 600 mg every three weeks.

190. The use of any one of claims 185-189, wherein the anti-TIGIT antagonist antibody comprises the following hypervariable regions (HVRs):
an HVR-H1 sequence comprising the amino acid sequence of SNSAAWN (SEQ ID NO: 1);
an HVR-H2 sequence comprising the amino acid sequence of KTYYRFKWYSDYAVSVKG (SEQ ID NO: 2);
an HVR-H3 sequence comprising the amino acid sequence of ESTTYDLLAGPFDY (SEQ ID NO: 3);
an HVR-L1 sequence comprising the amino acid sequence of KSSQTVLYSSNNKKYLA (SEQ ID NO: 4);
an HVR-L2 sequence comprising the amino acid sequence of WASTRES (SEQ ID NO: 5); and
an HVR-L3 sequence comprising the amino acid sequence of QQYYSTPFT (SEQ ID NO: 6).

191. The use of claim 190, wherein the anti-TIGIT antagonist antibody further comprises the following light chain variable region framework regions (FRs):
an FR-L1 comprising the amino acid sequence of DIVMTQSPDSLAVSLGERATINC (SEQ ID NO: 7);
an FR-L2 comprising the amino acid sequence of WYQQKPGQPPNLLIY (SEQ ID NO: 8);
an FR-L3 comprising the amino acid sequence of GVPDRFSGSGSGTDFTLTISSLQAEDVAVYYC (SEQ ID NO: 9); and
an FR-L4 comprising the amino acid sequence of FGPGTKVEIK (SEQ ID NO: 10).

192. The use of claim 190, wherein the anti-TIGIT antagonist antibody further comprises the following heavy chain variable region FRs:
an FR-H1 comprising the amino acid sequence of X₁VQLQQSGPGLVKPSQTLSLTCAISGDSVS (SEQ ID NO: 11), wherein X₁ is Q or E;
an FR-H2 comprising the amino acid sequence of WIRQSPSRGLEWLG (SEQ ID NO: 12);
an FR-H3 comprising the amino acid sequence of RITINPDTSKNQFSLQLNSVTPEDTAVFYCTR (SEQ ID NO: 13); and
an FR-H4 comprising the amino acid sequence of WGQGTLVTVSS (SEQ ID NO: 14).

193. The use of claim 192, wherein X₁ is Q.

194. The use of claim 192, wherein X₁ is E.

195. The use of any one of claims 190-194, wherein the anti-TIGIT antagonist antibody comprises:
(a) a heavy chain variable (VH) domain comprising an amino acid sequence having at least 95% sequence identity to the amino acid sequence of SEQ ID NO: 17 or 18;
(b) a light chain variable (VL) domain comprising an amino acid sequence having at least 95% sequence identity to the amino acid sequence of SEQ ID NO: 19; or
(c) a VH domain as in (a) and a VL domain as in (b).

196. The use of any one of claims 185-195, wherein the anti-TIGIT antagonist antibody comprises:
(a) a heavy chain variable (VH) domain comprising an amino acid sequence having at least 95% sequence identity to the amino acid sequence of SEQ ID NO: 17 or 18;
(b) a light chain variable (VL) domain comprising an amino acid sequence having at least 95% sequence identity to the amino acid sequence of SEQ ID NO: 19; or
(c) a VH domain as in (a) and a VL domain as in (b).

197. The use of any one of claims 185-196, wherein the anti-TIGIT antagonist antibody is a monoclonal antibody.

198. The use of claim 197, wherein the anti-TIGIT antagonist antibody is a human antibody.

199. The use of any one of claims 185-198, wherein the anti-TIGIT antagonist antibody is a full-length antibody.

200. The use of any one of claims 185-192 and 194-199, wherein the anti-TIGIT antagonist antibody is tiragolumab.

201. The use of any one of claims 185-198, wherein the anti-TIGIT antagonist antibody is an antibody fragment that binds TIGIT selected from the group consisting of Fab, Fab′, Fab′-SH, Fv, single chain variable fragment (scFv), and (Fab′)₂ fragments.

202. The use of any one of claims 185-201, wherein the anti-TIGIT antagonist antibody is an IgG class antibody.

203. The use of claim 202, wherein the IgG class antibody is an IgG1 subclass antibody.

204. The use of any one of claims 185-203, wherein the anti-PD-L1 antagonist antibody is to be administered to the subject at a fixed dose of about 1200 mg every three weeks.

205. The use of any one of claims 185-204, wherein the anti-PD-L1 antagonist antibody is atezolizumab (MPDL3280A), YW243.55.S70, MSB0010718C, MDX-1105, or MEDI4736.

206. The use of claim 205, wherein the anti-PD-L1 antagonist antibody is atezolizumab.

207. The use of any one of claims 185-204, wherein the anti-PD-L1 antagonist antibody comprises the following HVRs:
an HVR-H1 sequence comprising the amino acid sequence of GFTFSDSWIH (SEQ ID NO: 20);
an HVR-H2 sequence comprising the amino acid sequence of AWISPYGGSTYYADSVKG (SEQ ID NO: 21);
an HVR-H3 sequence comprising the amino acid sequence of RHWPGGFDY (SEQ ID NO: 22);
an HVR-L1 sequence comprising the amino acid sequence of RASQDVSTAVA (SEQ ID NO: 23);
an HVR-L2 sequence comprising the amino acid sequence of SASFLYS (SEQ ID NO: 24); and
an HVR-L3 sequence comprising the amino acid sequence of QQYLYHPAT (SEQ ID NO: 25).

208. The use of claim 207, wherein the anti-PD-L1 antagonist antibody comprises:
(a) a heavy chain variable (VH) domain comprising an amino acid sequence having at least 95% sequence identity to the amino acid sequence of SEQ ID NO: 26;
(b) a light chain variable (VL) domain comprising an amino acid sequence having at least 95% sequence identity to the amino acid sequence of SEQ ID NO: 27; or
(c) a VH domain as in (a) and a VL domain as in (b).

209. The use of any one of claims 185-208, wherein the anti-PD-L1 antagonist antibody comprises:
a VH domain comprising the amino acid sequence of SEQ ID NO: 26; and
a VL domain comprising the amino acid sequence of SEQ ID NO: 27.

210. The use of any one of claims 207-209, wherein the anti-PD-L1 antagonist antibody is a monoclonal antibody.

211. The use of claim 210, wherein the anti-PD-L1 antagonist antibody is a humanized antibody.

212. The use of claim 210 or 211, wherein the anti-PD-L1 antagonist antibody is a full-length antibody.

213. The use of any one of claims 207-211, wherein the anti-PD-L1 antagonist antibody is an antibody fragment that binds PD-L1 selected from the group consisting of Fab, Fab′, Fab′-SH, Fv, single chain variable fragment (scFv), and (Fab′)₂ fragments.

214. The use of any one of claims 207-213, wherein the anti-PD-L1 antagonist antibody is an IgG class antibody.

215. The use of claim 214, wherein the IgG class antibody is an IgG1 subclass antibody.

216. The use of any one of claims 185-215, wherein the anti-TIGIT antagonist antibody is to be administered to the subject at a fixed dose of about 600 mg of every three weeks and the anti-PD-L1 antagonist antibody is to be administered to the subject at a fixed dose of about 1200 mg every three weeks.

217. The use of any one of claims 185-216, wherein the length of each of the one or more dosing cycles is 21 days.

218. The use of any one of claims 185-217, wherein the anti-TIGIT antagonist antibody and anti-PD-L1 antagonist antibody are to be administered to the subject on about Day 1 of each of the one or more dosing cycles.

219. The use of any one of claims 185-218, wherein the anti-TIGIT antagonist antibody is to be administered to the subject before the anti-PD-L1 antagonist antibody.

220. The use of claim 219, wherein a first observation period is to follow administration of the anti-TIGIT antagonist antibody and second observation period is to follow administration of the anti-PD-L1 antagonist antibody.

221. The use of claim 220, wherein the first observation period and the second observation period are each between about 30 minutes to about 60 minutes in length.

222. The use of any one of claims 185-218, wherein the anti-PD-L1 antagonist antibody is to be administered to the subject before the anti-TIGIT antagonist antibody.

223. The use of claim 222, wherein a first observation period is to follow administration of the anti-PD-L1 antagonist antibody and second observation period is to follow administration of the anti-TIGIT antagonist antibody.

224. The use of claim 223, wherein the first observation period and the second observation period are each between about 30 minutes to about 60 minutes in length.

225. The use of any one of claims 185-218, wherein the anti-TIGIT antagonist antibody is to be administered to the subject simultaneously with the anti-PD-L1 antagonist antibody.

226. The use of any one of claims 185-225, wherein the anti-TIGIT antagonist antibody and anti-PD-L1 antagonist antibody are to be administered to the subject intravenously.

227. The use of claim 226, wherein the anti-TIGIT antagonist antibody is to be administered to the subject by intravenous infusion over 60±10 minutes.

228. The use of claim 226 or 227, wherein the anti-PD-L1 antagonist antibody is to be administered to the subject by intravenous infusion over 60±15 minutes.

229. The use of any one of claims 185-228, wherein a tumor sample obtained from the subject has been determined to have a detectable expression level of PD-L1.

230. The use of claim 229, wherein the detectable expression level of PD-L1 is a detectable protein expression level of PD-L1.

231. The use of claim 230, wherein the detectable protein expression level of PD-L1 has been determined by an immunohistochemical (IHC) assay.

232. The use of claim 231, wherein the IHC assay uses anti-PD-L1 antibody 22C3, SP142, SP263, or 28-8.

233. The use of claim 232, wherein the IHC assay uses anti-PD-L1 antibody 22C3.

234. The use of claim 233, wherein the tumor sample has been determined to have a tumor proportion score (TPS) of greater than, or equal to, 1%.

235. The use of claim 234, wherein the TPS is greater than, or equal to, 1% and less than 50%.

236. The use of claim 234, wherein the TPS is greater than, or equal to, 50%.

237. The use of claim 232, wherein the IHC assay uses anti-PD-L1 antibody SP142.

238. The use of claim 237, wherein the tumor sample has been determined to have a detectable expression level of PD-L1 in greater than, or equal to, 1% of the tumor cells in the tumor sample.

239. The use of claim 238, wherein the tumor sample has been determined to have a detectable expression level of PD-L1 in greater than, or equal to, 1% and less than 5% of the tumor cells in the tumor sample.

240. The use of claim 238, wherein the tumor sample has been determined to have a detectable expression level of PD-L1 in greater than, or equal to, 5% and less than 50% of the tumor cells in the tumor sample.

241. The use of claim 238, wherein the tumor sample has been determined to have a detectable expression level of PD-L1 in greater than, or equal to, 50% of the tumor cells in the tumor sample.

242. The use of any one of claims 237-241, wherein the tumor sample has been determined to have a detectable expression level of PD-L1 in tumor-infiltrating immune cells that comprise greater than, or equal to, 1% of the tumor sample.

243. The use of claim 242, wherein the tumor sample has been determined to have a detectable expression level of PD-L1 in tumor-infiltrating immune cells that comprise greater than, or equal to, 1% and less than 5% of the tumor sample.

244. The use of claim 242, wherein the tumor sample has been determined to have a detectable expression level of PD-L1 in tumor-infiltrating immune cells that comprise greater than, or equal to, 5% and less than 10% of the tumor sample.

245. The use of claim 242, wherein the tumor sample has been determined to have a detectable expression level of PD-L1 in tumor-infiltrating immune cells that comprise greater than, or equal to, 10% of the tumor sample.

246. The use of claim 229, wherein the detectable expression level of PD-L1 is a detectable nucleic acid expression level of PD-L1.

247. The use of claim 246, wherein the detectable nucleic acid expression level of PD-L1 has been determined by RNA-seq, RT-qPCR, qPCR, multiplex qPCR or RT-qPCR, microarray analysis, SAGE, MassARRAY technique, ISH, or a combination thereof.

248. The use of any one of claims 185-247, wherein the lung cancer is a non-small cell lung cancer (NSCLC).

249. The use of claim 248, wherein the NSCLC is a squamous NSCLC.

250. The use of claim 248, wherein the NSCLC is a non-squamous NSCLC.

251. The use of any one of claims 248-250, wherein the NSCLC is a locally advanced unresectable NSCLC.

252. The use of claim 251, wherein the NSCLC is a Stage IIIB NSCLC.

253. The use of any one of claims 248-251, wherein the NSCLC is a recurrent or metastatic NSCLC.

254. The use of claim 253, wherein the NSCLC is a Stage IV NSCLC.

255. The use of claim 253 or 254, wherein the subject has not been previously treated for Stage IV NSCLC.

256. The use of any one of claims 185-255, wherein the subject does not have a sensitizing epidermal growth factor receptor (EGFR) gene mutation or anaplastic lymphoma kinase (ALK) gene rearrangement.

257. The use of any one of claims 185-256, wherein the subject does not have a pulmonary lymphoepithelioma-like carcinoma subtype of NSCLC.

258. The use of any one of claims 185-257, wherein the subject does not have an active EBV infection or a known or suspected chronic active EBV infection.

259. The use of any one of claims 185-258, wherein the subject is negative for EBV IgM or negative by EBV PCR.

260. The use of claim 259, wherein the subject is negative for EBV IgM and negative by EBV PCR.

261. The use of claim 259 or 260, wherein the subject is positive for EBV IgG or positive for EBNA.

262. The use of claim 261, wherein the subject is positive for EBV IgG and positive for EBNA.

263. The use of any one of claims 185-262, wherein the subject is negative for EBV IgG or negative for EBNA.

264. The use of claim 263, wherein the subject is negative for EBV IgG and negative for EBNA.

265. The use of any one of claims 185-264, wherein administration of the anti-TIGIT antagonist antibody and anti-PD-L1 antagonist antibody results in a clinical response.

266. The use of claim 265, wherein the clinical response is an increase in the objective response rate (ORR) of the subject as compared to a reference ORR.

267. The use of claim 266, wherein the reference ORR is the median ORR of a population of subjects who have received a treatment comprising an anti-PD-L1 antagonist antibody without an anti-TIGIT antagonist antibody.

268. The use of any one of claims 265-267, wherein the clinical response is an increase in the progression-free survival (PFS) of the subject as compared to a reference PFS time.

269. The use of any one of claims 265-268, wherein the reference PFS time is the median PFS time of a population of subjects who have received a treatment comprising an anti-PD-L1 antagonist antibody without an anti-TIGIT antagonist antibody.

270. Use of an anti-TIGIT antagonist antibody and atezolizumab in the manufacture of a medicament for use in a method of treating a subject having a NSCLC, wherein the method comprises administering to the subject one or more dosing cycles of the medicament, and wherein the medicament is formulated for administration of the anti-TIGIT antagonist antibody at a fixed dose of 600 mg every three weeks and atezolizumab at a fixed dose of 1200 mg every three weeks, and wherein the anti-TIGIT antagonist antibody comprises:
a VH domain comprising the amino acid sequence of SEQ ID NO: 17 or 18; and
a VL domain comprising the amino acid sequence of SEQ ID NO: 19.

271. Use of tiragolumab and atezolizumab in the manufacture of a medicament for use in a method of treating a subject having a NSCLC, wherein the method comprises administering to the subject one or more dosing cycles of the medicament, and wherein the medicament is formulated for administration of tiragolumab at a fixed dose of 600 mg every three weeks and atezolizumab at a fixed dose of 1200 mg every three weeks.

272. A kit comprising an anti-TIGIT antagonist antibody, an anti-PD-L1 antagonist antibody, and a package insert comprising instructions to administer the anti-TIGIT antagonist antibody and the anti-PD-L1 antagonist antibody to a subject having a lung cancer in accordance with the methods of any one of claims 1-86 and 192-194.
